(19) United States
(12) Patent Application Publication
(10) Pub. No.: US 2023/0389833 A1
(43) Pub. Date: Dec. 7, 2023

(54) SYSTEMS AND METHODS FOR MONITORING, DIAGNOSIS, AND DECISION SUPPORT FOR DIABETES IN PATIENTS WITH KIDNEY DISEASE

(71) Applicant: Dexcom, Inc., San Diego, CA (US)

(72) Inventors: Matthew L. JOHNSON, San Diego, CA (US); Qi AN, San Diego, CA (US); Rush BARTLETT, San Diego, CA (US); John PADERI, San Diego, CA (US)

(21) Appl. No.: 18/326,985

(22) Filed: May 31, 2023

Related U.S. Application Data
(60) Provisional application No. 63/365,702, filed on Jun. 1, 2022.
(60) Provisional application No. 63/376,673, filed on Sep. 22, 2022.
(60) Provisional application No. 63/387,078, filed on Dec. 12, 2022.
(60) Provisional application No. 63/377,332, filed on Sep. 27, 2022.
(60) Provisional application No. 63/403,568, filed on Sep. 2, 2022.
(60) Provisional application No. 63/403,582, filed on Sep. 2, 2022.

(51) Int. Cl. A61B 5/145; A61B 5/0205; G16H 50/30; G16H 50/70

(52) U.S. Cl. CPC A61B 5/14546; A61B 5/14532; G16H 50/70; G16H 50/30; A61B 5/02055

(57) ABSTRACT

Certain aspects provide a monitoring system comprising a continuous analyte sensor configured to generate analyte measurements associated with analyte levels of a patient, and a sensor electronics module coupled to the continuous analyte sensor and configured to receive and process the analyte measurements.

### CROSS-REFERENCE TO RELATED APPLICATIONS

This application claims priority to and benefit of U.S. Provisional Application No. 63/365,702, filed Jun. 1, 2022, and U.S. Provisional Application No. 63/376,673, filed Sep. 22, 2022, and U.S. Provisional Application No. 63/387,078, filed Dec. 12, 2022, and U.S. Provisional Application No. 63/377,332, filed Sep. 27, 2022, and U.S. Provisional Application No. 63/403,568, filed Sep. 2, 2022, and U.S. Provisional Application No. 63/403,582, filed Sep. 2, 2022, which are hereby assigned to the assignee hereof and hereby expressly incorporated by reference in their entirety as if fully set forth below and for all applicable purposes.

### BACKGROUND

The kidney is responsible for many critical functions within the human body including, filtering waste and excess fluids, which are excreted in the urine, and removing acid that is produced by the cells of the body to maintain a healthy balance of water, salts, and minerals (e.g., such as sodium, calcium, phosphorus, and potassium) in the blood. In other words, the kidney plays a major role in homeostasis by renal mechanisms that transport and regulate water, salt, and mineral secretion, reabsorption, and excretion. Further, kidneys secrete renin (e.g., angiotensinogenase), which forms part of the renin-angiotensin-aldosterone system (RAAS) that mediates extracellular fluid and arterial vasoconstriction (e.g., blood pressure). More specifically, high blood pressure (e.g., hypertension) can be regulated through RAAS inhibitors such as angiotensin-converting enzyme (ACE) inhibitors and angiotensin receptor blockers (ARBs). Should the kidney become diseased or injured, the impairment or loss of these functions can cause significant damage to the human body.

Kidney disease occurs when the kidney becomes diseased or injured. Kidney disease is generally classified as either acute or chronic based upon the duration of the disease. Acute kidney injury (AKI) (also referred to as “acute renal failure”) is usually caused by an event that leads to kidney malfunction, such as dehydration, blood loss from major surgery or injury, and/or the use of medicines. On the other hand, chronic kidney disease (CKD) is usually caused by a long-term disease, such as high blood pressure or diabetes, which slowly damages the kidneys and reduces their function over time.

Conventional kidney disease diagnostic methods and systems include electrocardiogram (ECG) monitoring, albumin-to-creatinine ratio (ACR) tests, glomerular filtration rate (GFR) tests, and blood tests for monitoring potassium levels of a patient. CKD is divided into five stages based on the severity of kidney dysfunction, as measured by the various methods and systems. Kidney disease in stages 1-3a is mild to moderate kidney dysfunction. Kidney disease in stages 3b-5 is moderate to severe kidney dysfunction. End stage renal disease (ESRD) is total kidney dysfunction or kidney failure.

The GFR method for diagnosis and staging of kidney disease represents the flow rate of filtered fluid through the kidney. Creatinine clearance rate is the volume of blood plasma that is cleared of creatinine per unit of time and is used to approximate the GFR. GFR can be measured (e.g., measured GFR (mGFR)) with gold standard methods (e.g., assessed from urinary or plasma clearance measurements of exogenous filtration markers) or estimated (e.g., eGFR) with formulas (e.g., from measured serum levels of exogenous filtration markers using formulas). eGFR provides a more convenient and rapid analysis for evaluating kidney function.

In some cases, when CKD is left untreated, elevated potassium levels of a patient with CKD may lead to hyperkalemia, while lower potassium levels of a patient with CKD may lead to hypokalemia. Hyperkalemia is the medical term that describes a potassium level in the blood that is higher than normal (e.g., higher than normal blood potassium levels between 3.6 and 5.2 millimoles per liter (mmol/L)). Hyperkalemia increases the risk of cardiac arrhythmia episodes and sudden death. On the other hand, hypokalemia is the medical term that describes a potassium level in the blood that is lower than normal. In particular, CKD patients may develop hypokalemia due to gastrointestinal potassium loss from diarrhea or vomiting or renal potassium loss from non-potassium-sparing diuretics (e.g., diuretics used to increase the amount of fluid passed from the body in urine, without regard for the amount of potassium being lost from the body in the urine). Severe hypokalemia and hyperkalemia may lead to severe symptoms of respiratory failure, sudden cardiac death, or other mortality-driven event.

The kidney also plays an important role in the regulation of blood glucose. The kidney raises blood glucose levels by generating glucose, via gluconeogenesis, and releasing glucose into the blood. The kidney also lowers blood glucose levels by reabsorbing glucose at the proximal tubule of the kidney. Additionally, the kidney uses glucose as an energy source.

Glucose is a simple sugar (e.g., a monosaccharide). Glucose can be both ingested, as well as produced in the body from protein, fat, and carbohydrates. Serum glucose is maintained at healthy levels (e.g., glucose homeostasis) through several mechanisms. High blood glucose (i.e., hyperglycemia) is reduced by insulin and cleared by the kidney. Additionally, the kidney both consumes glucose and filters glucose from the body, as well as generates glucose (e.g., through gluconeogenesis) and reabsorbs filtered glucose. Therefore, as kidney function declines, glucose consumption, filtration, generation, and reabsorption declines, which may lead to increasing glucose levels in the body at rest and after consuming a meal, for example. Low blood glucose (i.e., hypoglycemia) is raised by gluconeogenesis in the kidney and liver.

Increasing blood glucose levels stimulate insulin release. Insulin causes the cells to take in glucose, thereby reducing serum (e.g., extracellular) glucose levels to maintain glucose homeostasis. Insulin also stimulates potassium uptake by cells, thereby reducing serum potassium levels. In some cases, where glucose levels of a patient are increased and rate(s) of change of glucose levels in the patient's body are high, excess insulin may be produced thereby causing excess movement of potassium intracellularly. On the other hand, where glucose levels of a patient are decreased and rate(s) of change of glucose levels in the patient's body are low, there may be less insulin secretion. Low insulin may lead to limited access of glucose and potassium by the cells; thus, extracellular glucose and potassium levels may increase.

Gluconeogenesis is the formation of glucose from precursor molecules (e.g., lactate, glycerol, and/or amino acids). Glucose is formed in the kidney and liver, and then released into circulation. Gluconeogenesis is a mechanism to maintain glucose homeostasis by preventing low blood glucose (i.e., hypoglycemia). As kidney function declines, gluconeogenesis in the kidney declines, and thus limits the kidney's ability to react to falling blood glucose.

Diabetes mellitus is a disorder in which the pancreas cannot create insulin (Type I or insulin dependent) and/or in which insulin is not effective (Type 2 or non-insulin dependent). In the diabetic state, the patient suffers from high blood sugar, which causes an array of physiological derangements (e.g., kidney failure, skin ulcers, or bleeding into the vitreous of the eye) associated with the deterioration of small blood vessels. A hypoglycemic reaction (i.e., low blood sugar) can be induced by an inadvertent overdose of insulin, or after a normal dose of insulin or glucose lowering agent, or insufficient food intake. Treatment for diabetes requires maintenance of glucose homeostasis. Glucose levels may be controlled through a variety of medications, including exogenous insulin.

In some cases, a patient may suffer from insulin resistance. Insulin resistance occurs when cells in the patient's muscles, fat, and liver do not respond well to insulin. Accordingly, glucose metabolism, as well as potassium movement intracellularly may be impaired. As a result, the patient's pancreas makes more insulin to help glucose and insulin enter the patient's cells. Further, the effect of insulin resistance on glucose metabolism may be different for different patients.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system that may be used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIG. 4 is an example workflow for generating, using a trained machine learning model, a diabetes prediction for a patient with kidney disease, and providing one or more recommendations for treatment for the user based on the generated diabetes prediction, according to certain embodiments of the present disclosure.

FIG. 5 is a flow diagram illustrating an example method for providing decision support using a continuous analyte sensor including, at least, a continuous glucose sensor, in accordance with some example aspects of the present disclosure.

FIG. 6 is a flow diagram depicting a method for training machine learning models to provide predictions associated with diabetes and kidney disease, according to certain embodiments of the present disclosure.

FIG. 7 is a block diagram depicting a computing device configured to perform the operations of FIGS. 4, 5, and/or 6, according to certain embodiments disclosed herein.

FIGS. 8A-8B depict exemplary enzyme domain configurations for a continuous multi-analyte sensor, according to certain embodiments of the present disclosure.

FIGS. 8C-8D depict exemplary enzyme domain configurations for a continuous multi-analyte sensor, according to certain embodiments of the present disclosure.

FIG. 8E depicts an exemplary enzyme domain configuration for a continuous multi-analyte sensor, according to certain embodiments of the present disclosure.

FIGS. 9A-9B depict alternative views of an exemplary dual electrode enzyme domain configuration for a continuous multi-analyte sensor, according to certain embodiments of the present disclosure.

FIGS. 9C-9D depict alternative views of an exemplary dual electrode enzyme domain configuration for a continuous multi-analyte sensor, according to certain embodiments of the present disclosure.

FIG. 9E depicts an exemplary dual electrode configuration for a continuous multi-analyte sensor, according to certain embodiments of the present disclosure.

FIG. 10A depicts an exemplary enzyme domain configuration for a continuous multi-analyte sensor, according to certain embodiments of the present disclosure.

FIGS. 10B-10C depict alternative exemplary enzyme domain configurations for a continuous multi-analyte sensor, according to certain embodiments of the present disclosure.

FIG. 11 depicts an exemplary enzyme domain configuration for a continuous multi-analyte sensor, according to certain embodiments of the present disclosure.

FIGS. 12A-12D depict alternative views of exemplary dual electrode enzyme domain configurations G1-G4 for a continuous multi-analyte sensor, according to certain embodiments of the present disclosure.

FIGS. 13A-13B schematically illustrate an example configuration and component of a device for measuring an electrophysiological signal and/or concentration of a target ion in a biological fluid in vivo, according to certain embodiments of the present disclosure.

FIG. 14 schematically illustrates additional example configurations and component of a device for measuring an electrophysiological signal and/or a concentration of a target ion in a biological fluid in vivo, according to certain embodiments of the present disclosure.

FIGS. 15A-15C schematically illustrate example configurations and components of a device for measuring an electrophysiological signal and/or concentration of a target analyte in a biological fluid in vivo, according to certain embodiments of the present disclosure.

FIG. 16 is a diagram depicting an example continuous analyte monitoring system configured to measure target ions and/or other analytes as discussed herein, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

One of the biggest risks associated with chronic kidney disease (CKD) is hypoglycemia. 1.7% of hospitalizations annually are due to hypoglycemia for early-stage CKD (CKD<stage 3). 3.6% of end stage renal disease (ESRD) related hospitalizations are due to hypoglycemia with a 30% mortality rate. Specifically, reduced glucose homeostasis increases the risk of hypoglycemic events. Hypoglycemic events in kidney disease result from decreased insulin clearance, impaired glucose clearance, and impaired kidney gluconeogenesis. For ESRD-related hospitalizations, dialysis is also a compounding factor in reduced glucose homeostasis. There is need for improved diabetes diagnosis and glucose management for patients with kidney disease.

Kidney dysfunction decreases the body's ability to maintain glucose homeostasis through decreased insulin clearance, impaired gluconeogenesis, and impaired glucose clearance. Insulin clearance is decreased with decreasing kidney function. The kidney cannot adequately filter and remove (i.e., clear) insulin from circulation. Thus, both endogenous and exogenous insulin may have a prolonged and/or pronounced effect. This increases risk of hypoglycemia in a patient with kidney dysfunction. As kidney dysfunction progresses, insulin may have an even more prolonged and/or pronounced effect, which further increases the risk of hypoglycemia for a patient with worsening CKD. For example, for a patient with worsening CKD, an insulin dose that reduces blood glucose to a healthy range at one point, may at a later point result in hypoglycemia.

Additionally, gluconeogenesis in the kidney also declines as a result of CKD. Thus, the kidney's ability to react to falling blood glucose or hypoglycemia is reduced. Furthermore, due to reduced insulin clearance, insulin remains and circulates for longer, which further reduces glucose levels. In such cases, the kidney's response to falling glucose, gluconeogenesis, is ineffective and glucose levels continue to drop with insulin remaining for a longer amount of time, resulting in hypoglycemia. In addition, glucose clearance is impaired in a patient with CKD because the kidney cannot filter, reabsorb, and consume blood glucose as effectively. Thus, high glucose levels will not be reduced to adequate levels, potentially causing hyperglycemia.

Current methods for providing decision support for diabetes, including diagnosis, monitoring, and treatment are unreliable for patients with diabetes and CKD, because such methods fail to account for the effects of kidney dysfunction on blood glucose. Specifically, one methodology for diabetes diagnosis, A1C, is especially unreliable in patients with advanced stages of CKD (CKD>stage 3). A1C is a measurement of the glycation of hemoglobin found in red blood cells. It is a percentage of glycation-modified hemoglobin based on assumed red blood cell half-life. A1C thus summarizes the duration of high blood glucose during the life of the red blood cells. However, in patients with kidney dysfunction, red blood cell half-life is decreased. This shorter red blood cell half-life means a clinically measured A1C is based on an assumed red blood cell half-life that is inaccurate. More particularly, the clinically measured A1C for a patient with CKD will be lower than the patient's actual A1C. For example, a patient with CKD may have a glycated A1C level in a healthy range (e.g., 5.5%) while actual blood glucose levels may be elevated (e.g., 200 mg/dL). This can be especially pronounced as a difference when it comes to post-prandial times that patient's glucose levels could be significantly elevated but not so easily known through some diagnostic methods such as a fasting glucose test. Additionally, for patients with severe CKD (estimated GFR<30 mL/min/1.73 m2) shortened red blood cell half-life is well-documented, especially in those patients receiving dialysis and erythropoietin-stimulating agents. Clinical practice guidelines for physicians treating patients with CKD even recommend maintaining a slightly elevated A1C as a method to lessen the chances of a severe hypoglycemic episode in this patient population, despite literature demonstrating that for a patient with less glycemic control, kidney disease progression will accelerate faster than for a patient with better glycemic control. Thus, A1C is not a reliable estimation of blood glucose in patients with CKD.

Therefore, patients with CKD can remain undiagnosed with diabetes until diabetes has progressed to a severity that conventional methods may detect it. These patients will suffer from reduced glucose homeostasis, including increased risk of adverse glycemic events, such as hypoglycemia and hyperglycemia.

For a patient with kidney dysfunction, the diagnostic level of A1C could be adjusted down to account for the effects of kidney disease on A1C. For example, a patient with known kidney dysfunction would have an artificially low A1C whereby a clinician may diagnose diabetes at a lower thresholder (e.g., 6.0% instead of 6.5%) to account for the effects of kidney dysfunction on red blood cell half-life in calculating clinical A1C. However, this method is still prone to error because actual red blood cell half-life in patients with kidney dysfunction is unknown without additional analysis. Additionally, impaired clearance of waste products such as urea can speed up the glycation process, and thus lead to a higher A1C value as compared to a healthy individual. Thus, the adjusted A1C will still be calculated based on an inaccurate assumption without taking patient-to-patient variability into account for a personalized assessment.

Additionally, for patients with CKD, blood glucose may have greater fluctuations due to kidney dysfunction. Greater blood glucose fluctuations result in increased glycemic variability. High glycemic variability may be due to higher and/or longer elevated glucose levels as well as lower and/or longer depressed glucose levels. Patients with CKD may experience greater glycemic variability because the kidney is less effective at reducing high glucose levels and raising low glucose levels. Thus, kidney dysfunction impairs glucose homeostasis and increases glycemic variability.

Additionally, for patients with advanced CKD on dialysis, blood glucose may have greater variability in fluctuations. The effect of dialysis, the concentration of carbohydrate or salts in the dialysate solution, the membrane charge or filtration size, dialysis filtration rate, and other dialysis parameters may add to the fluctuation variability as glucose is filtered out or added into the bloodstream during dialysis. In addition, although insulin is a large enough molecule to not be easily cleared through a dialysis membrane, insulin molecules may stick to the membrane to be partially cleared through dialysis. The loss of insulin during dialysis may reduce the amount of available insulin during and immediately after the dialysis procedure, which may result in a glycemic spike following a dialysis procedure.

Impaired glucose homeostasis and increased glycemic variability increase risk of hyperglycemia and/or hypoglycemia. Therefore, patients with CKD will be at increased risk of hyperglycemia and/or hypoglycemia. Also, a patient with kidney dysfunction will have atypical glucose trends compared to similarly situated patients without kidney dysfunction. For example, a patient without kidney dysfunction may consume a meal and experience a resulting increase in glucose levels, while a patient with kidney dysfunction who consumes the same meal may experience a lower resulting increase in glucose levels. Because kidney dysfunction also impacts insulin clearance, atypical glucose trends may also appear following administration of insulin. For example, a patient without kidney dysfunction may consume a meal and administer a dose of insulin and experience a resulting glucose trend, while a patient with kidney dysfunction who consumes the same meal and insulin dose may experience a different glucose trend.

Therefore, even once a patient with CKD has been diagnosed with diabetes, conventional treatment methods may not account for the impact of CKD on their diabetes and ability to maintain glucose homeostasis. Conventional methods and systems used for assessing, monitoring, and treating diabetes in a patient with kidney disease fail to account for the interactions between kidney disease and diabetes. Current diabetes monitoring and treatments utilize continuous analyte monitoring systems, including, at least, a continuous glucose sensor.

Some examples of a continuous glucose monitor include a glucose monitoring sensor. In some embodiments, glucose monitoring sensor is an implantable sensor, such as described with reference to U.S. Pat. No. 6,001,067 and U.S. Patent Publication No. US-2011-0027127-A1. In some embodiments, the glucose monitoring sensor is a transcutaneous sensor, such as described with reference to U.S. Patent Publication No. US-2006-0020187-A1. In yet other embodiments, the glucose monitoring sensor is a dual electrode analyte sensor, such as described with reference to U.S. Patent Publication No. US-2009-0137887-A1. In still other embodiments, the glucose monitoring sensor is configured to be implanted in a host vessel or extracorporeally, such as the sensor described in U.S. Patent Publication No. US-2007-0027385-A1. These patents and publications are incorporated herein by reference in their entirety.

As used herein, the term “continuous” may mean fully continuous, semi-continuous, periodic, etc. Such continuous monitoring of analytes is advantageous in diagnosing and staging a disease given the continuous measurements provide continuously up to date measurements as well as information on the trend and rate of analyte change over a continuous period. Such information may be used to make more informed decisions in the assessment of glucose homeostasis and treatment of diabetes.

Overall, existing diagnostic methods suffer from a first technical problem of inaccurate and unreliable methods of diagnosing diabetes in patients with kidney dysfunction because of the impacts of kidney dysfunction on glucose homeostasis, including the impact on red blood cell half-life and increased glycemic variability.

Additionally, existing monitoring and treatment methods also suffer from a second technical problem of failing to account for the implications of kidney disease on diabetes and/or glucose homeostasis when providing health and intervention strategies.

As a result of these technical problems, diagnosing diabetes, hyperglycemia, and/or hypoglycemia, or a risk thereof, as well as maintaining glucose homeostasis in patients with kidney disease using conventional techniques may not only be inaccurate but also impossible, which, in some cases, might prove to be life threatening for a patient with such disease(s). Specifically, predicting the presence of diabetes in patients with kidney dysfunction in a personalized manner with reasonable accuracy may be necessary due to the conflation, interaction, and co-presence of diabetes and kidney disease. Further, predicting risk of hyperglycemia and/or hypoglycemia and corresponding atypical glucose trends may be necessary given both hypoglycemia and hyperkalemia may lead to severe, and life-threatening conditions. Thus, improved methods for detecting and predicting diabetes in a patient with kidney disease based on the interplay between such conditions in a patient is desired.

Accordingly, certain embodiments described herein provide a technical solution to the technical problems described above by providing an improved decision support and diagnostic system that is configured to account for the effects of kidney disease on glucose homeostasis to provide more accurate and effective decision support to a patient with both diabetes and kidney disease. As discussed in more detail herein, decision support may be provided in the form of risk assessment, diagnosis, staging, alerts, alarms, and/or recommendations for treatment of diabetes in patients with kidney dysfunction, as described in more detail herein. As used herein, risk assessment may refer to the evaluation of risks associated with the presence and progression of diabetes, evaluation or estimation of diabetes reaching a more advanced stage, mortality risk, a risk of being diagnosed with one or more other diseases, a risk of experiencing one or more symptoms, and the like.

In certain embodiments, the continuous analyte monitoring system may provide decision support to a patient based on a variety of collected data, including analyte data, patient information, secondary sensor data (e.g., non-analyte data), etc. For example, the analyte data may include continuously monitored glucose data in addition to other continuously monitored analyte data, such as potassium, lactate, pyruvate, insulin, and ketones.

Certain embodiments of the present disclosure provide techniques and systems for using measurements associated with other analyte sensor data, the secondary sensor data, and/or other patient information, in conjunction with glucose levels as further described below. As described above, the collected data also includes patient information, which may include information related to age, gender, family history of diabetes, kidney disease, family history of kidney disease, other health conditions, etc. Secondary sensor data may include accelerometer data, heart rate data (ECG, HRV, HR, etc.), temperature, blood pressure, or any other sensor data other than analyte data.

According to certain embodiments of the present disclosure, the decision support system presented herein is configured to provide a diagnosis for patients with, or at risk of, diabetes as well as disease decision support to assist the patient in managing their diabetes and kidney disease, or risk thereof. Providing diabetes decision support may involve using large amounts of collected data, including for example, analyte data, patient information, and secondary sensor data mentioned above, to (1) automatically detect abnormal patterns associated with various glucose metrics (2) assess the presence and severity of diabetes, (3) risk stratify patients to identify those patients with a high risk of diabetes, (4) identify risks (e.g., mortality risk, hyperglycemia risk, hypoglycemia risk, dementia, end-stage renal disease, CKD stages 3 to 4, heart failure, myocardial infarction, chronic obstructive pulmonary disease COPD, cirrhosis, proliferative retinopathy, etc.) associated with a current diabetes diagnosis, (5) make patient-specific treatment decisions or recommendations for managing glucose homeostasis and kidney disease, and (6) provide information on the effect of an intervention (e.g., an effect of a lifestyle change of the patient, an effect of a surgical procedure, an effect of the patient taking new medication, an effect of the patient taking insulin, etc.). In other words, the decision support system presented herein may offer information to direct and help improve care for patients with kidney dysfunction and with, and at risk of, reduced glucose homeostasis.

According to certain embodiments of the present disclosure, the decision support system presented herein is configured to predict the risk or likelihood of at least one atypical glucose trend occurring in-real time or within a specified period of time for a patient (e.g., a time period in the future, such as the next few second, minutes, hours, days, etc.). In particular, the decision support system presented herein may be configured to predict the risk or likelihood of a patient, e.g., with kidney disease, experiencing (in real-time or at a later time) hyperglycemia and/or hypoglycemia as well as the effectiveness of various treatment options. As discussed, patients with kidney disease and diabetes may experience hyperglycemia, hypoglycemia and/or other glucose trends (e.g., glucose variability, time in range (TIR), etc.) differently than patients with only diabetes. Further, treatment effectiveness may be different in patients with kidney disease and diabetes as opposed to patients with only diabetes. Therefore, recognition of atypical glucose trends and effective treatment options for patients with diabetes and kidney disease may be critical to successful management of glucose in patients with kidney disease

In certain embodiments, the decision support system described herein may use various algorithms or artificial intelligence (AI) models, such as machine-learning models, trained based on patient-specific data and/or population data to provide real-time decision support to a patient based on the collected information about the patient. For example, certain aspects are directed to algorithms and/or machine-learning models designed to assess the presence and severity of diabetes in a patient. The algorithms and/or machine-learning models may be used in combination with one or more continuous analyte sensors, including at least a continuous glucose sensor, to provide real-time diabetes assessment. In particular, the algorithms and/or machine-learning models may take into account parameters, such as glucose metrics of the patient over time, and physiological parameters of a patient, such as kidney disease and stage, when diagnosing, monitoring, and providing decision support for diabetes.

Based on these parameters, the algorithms and/or machine-learning models may provide a risk assessment of diabetes, type, and/or severity, a risk assessment of CKD and its progression, type, and severity as well as various types of decision support. The algorithms and/or machine-learning models may take into consideration population data, personalized patient-specific data, or a combination of both when diagnosing diabetes for a patient with kidney disease.

According to certain embodiments, prior to deployment, the machine learning models are trained with training data, e.g., including user-specific data and/or population data. As described in more detail herein, the population data may be provided in a form of a dataset including data records of historical patients with varying stages of kidney disease, varying types of diabetes, and/or experiencing different symptoms associated with reduced glucose homeostasis, and in some cases, diabetes. Each data record may be used as input into the machine learning models to optimize such models to generate accurate predictions associated with diabetes (e.g., predictions of diabetes presence, type of diabetes, severity of diabetes, etc.). The combination of a continuous analyte monitoring system with machine learning models and/or algorithms for (1) predicting the presence and/or severity of diabetes in patients with kidney disease and (2) providing decision support (e.g., predicting optimal treatment, providing diet and exercise recommendations, etc.) for the management of glucose for a patient with kidney disease. For example, the decision support system may be used to provide earlier alert of diabetes, reduced glucose homeostasis, and/or deliver information about glucose levels. Maintaining glucose levels reduces the risk of severe health outcomes, especially for patients with kidney disease. For example, detection and support for glucose management for patients with kidney disease, reduces risk of hospitalization, complications, and death, in some cases. In addition, glucose metrics and atypical glucose trends provided by the continuous analyte monitoring system may be used as input into the machine learning models and/or algorithms to triage patients for more urgent care.

Through the combination of a continuous analyte monitoring system with machine learning models and/or algorithms, the decision support system described herein is configured to provide the necessary accuracy and reliability patients expect. For example, biases, human errors, and emotional influence may be minimized when assessing the presence and severity of diabetes in patients with kidney disease. Further, machine learning models and algorithms in combination with analyte monitoring systems may provide insight into patterns and/or trends of decreasing health of a patient, at least with respect to the kidney and/or glucose, which may have been previously missed. Accordingly, the decision support system described herein improves existing decision support systems and, more generally, the field of disease monitoring, diagnosis, and treatment.

### Example Decision Support System Including an Example Analyte Sensor

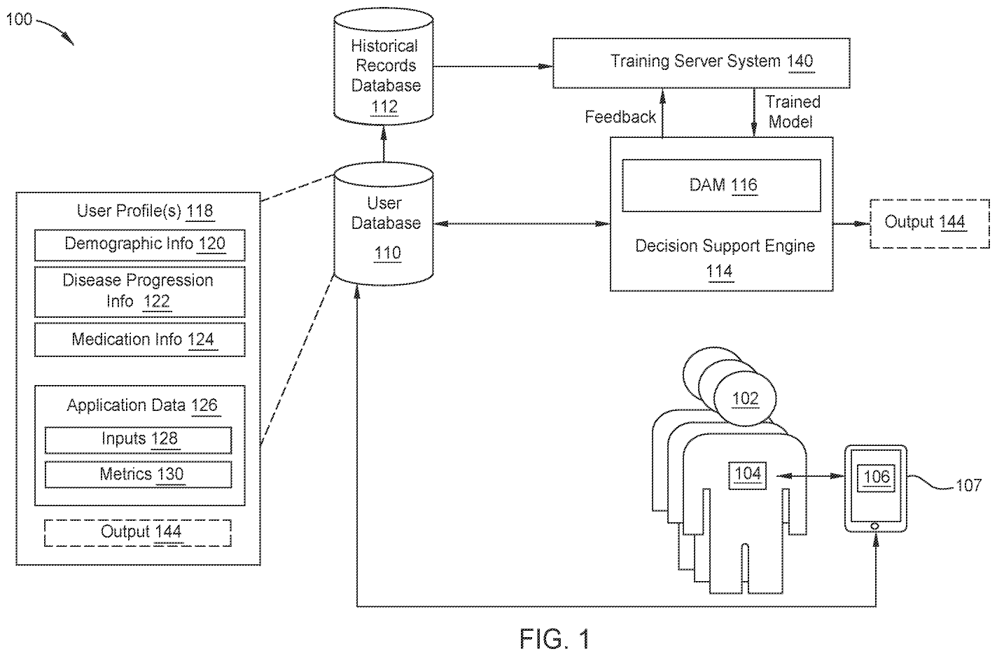

FIG. 1 illustrates an example decision support system 100 for (1) predicting the presence and/or severity of diabetes in patients with kidney disease and (2) providing decision support to patients with diabetes and/or kidney disease. Decision support system 100 is configured to provide decision support to users 102 (individually referred to herein as a user and collectively referred to herein as users), using a continuous analyte monitoring system 104, including, at least, a continuous glucose sensor. A user, in certain embodiments, may be the patient or, in some cases, the patient's caregiver. In certain embodiments, decision support system 100 includes continuous analyte monitoring system 104, a display device 107 that executes application 106, a decision support engine 114, a user database 110, a historical records database 112, a training server system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atom or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include glucose and potassium, and in some cases lactate, pyruvate, and ketone, other analytes listed, but not limited to, above may also be considered and measured by, for example, analyte monitoring system 104.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR may be in communication with decision support engine 114 (e.g., via a network) for performing the techniques described herein. The communication could come through a variety of network connection data configurations including but not limited to web API protocols, HL7, FHIR, EDI, XML, CDA, and others. These data communication configurations could be sent directly to the EMR, or through one or more intermediary systems including but not limited to an interface engine before entering the EMR system to then be displayed. Patient data communicated into the EMR via any of these other means could be matched to a patient record through probabilistic matching, manual human matching, or an EMPI or MPI (master patient index, electronic master patient index) to ensure that the data input to one system matches the patient information in another system. Data from an analyte device could also be matched with data from alternative devices or systems prior to being inputted into the EMR or data could be sent in the reverse direction into our historical records database 112, user database 110, and or decision support engine. These data transfers allow the system to perform optimized decision support through the means described herein. In particular, as described herein, decision support engine 114 may obtain data associated with a user, use the obtained data as input into one or more trained model(s), and output a prediction. In some cases, the EMR may provide the data to decision support engine 114 to be used as input into the one or more models. Further, in some cases, decision support engine 114, after making a prediction, may provide the output prediction to the EMR.

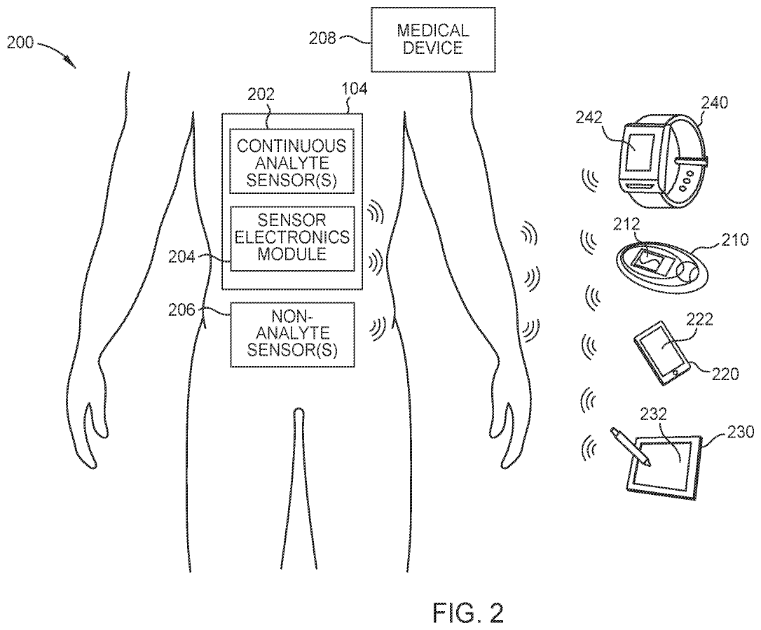

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application that is configured to receive and analyze analyte measurements from analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114 may provide decision support recommendations to the user via application 106. Decision support engine 114 provides decision support recommendations based on information included in user profile 118.

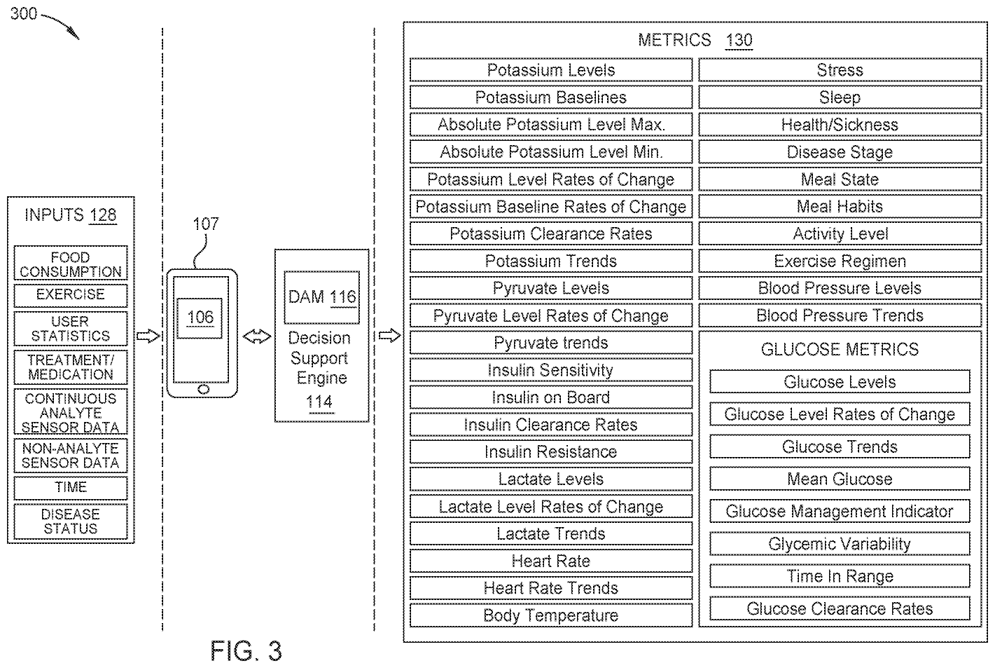

User profile 118 may include information collected about the user from application 106. For example, application 106 provides a set of inputs 128, including the analyte measurements received from continuous analyte monitoring system 104, that are stored in user profile 118. In certain embodiments, inputs 128 provided by application 106 include other data in addition to analyte measurements received from continuous analyte monitoring system 104. For example, application 106 may obtain additional inputs 128 through manual user input, one or more other non-analyte sensors or devices, other applications executing on display device 107, etc. Non-analyte sensors and devices include one or more of, but are not limited to, an insulin pump, an electrocardiogram (ECG) sensor or heart rate monitor, a blood pressure sensor, a sweat sensor, a respiratory sensor, a thermometer, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, metrics 130 may then be used by decision support engine 114 as input for providing guidance to a user. As shown, metrics 130 are also stored in user profile 118.

User profile 118 also includes demographic info 120, disease progression info 122, and/or medication info 124. In certain embodiments, such information may be provided through user input or obtained from certain data stores (e.g., electronic medical records (EMRs), etc.). In certain embodiments, demographic info 120 may include one or more of the user's age, body mass index (BMI), ethnicity, gender, etc. In certain embodiments, disease progression info 122 may include information about a disease of a user, such as whether the user has been previously diagnosed with acute kidney injury (AKI), chronic kidney disease (CKD), and/or diabetes, or have had a history of hyperkalemia, hypokalemia, hyperglycemia, hypoglycemia, etc. In certain embodiments, information about a user's disease may also include the length of time since diagnosis, the stage of disease, the level of disease control, level of compliance with disease management therapy, predicted kidney function, other types of diagnosis (e.g., heart disease, obesity) or measures of health (e.g., heart rate, exercise, stress, sleep, etc.), and/or the like. In certain embodiments, disease progression info 122 may be provided as an output of one or more predictive algorithms and/or trained models based on analyte sensor data generated, for example, through continuous analyte monitoring system 104.

In certain embodiments, medication info 124 may include information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels. In certain embodiments, medication information 124 may include information about consumption of one or more drugs known to control and/or improve glucose homeostasis. One or more drugs known to control and/or improve glucose homeostasis may include medications to lower blood glucose levels such as insulin, including rapid acting, and long-acting insulin, other glucose lowering medications, such as metformin, and the like. As described in more detail below, decision support system 100 may be configured to use medication information 124 to determine optimal insulin administration to be prescribed to different users. In particular, decision support system 100 may be configured to identify one or more optimal insulin administration based on the health of the patient, the patient's current condition, and/or effectiveness of insulin administration.

In certain embodiments, medication information 124 may include information about consumption of one or more drugs known to damage the kidney. One or more drugs known to damage the kidney may include nonsteroidal anti-inflammatory drugs (NSAIDS) such as ibuprofen (e.g., Advil, Motrin) and naproxen (e.g., Aleve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycdin, gentamicin, tobramycin, and amikacin, antiviral human immunodeficiency virus (HIV) medications, zoledronic acid (e.g., Zometa, Reclast), foscarnet, and the like.

In certain embodiments, medication information 124 may include information about consumption of one or more drugs known to control the complications of kidney disease. One or more drugs known to control the complications of kidney disease may include medications to lower blood pressure and preserve kidney function such as ACE inhibitors or angiotensin II receptor blockers, medications to treat anemia such as supplements of the hormone erythropoietin, medications used to lower cholesterol levels such as statins, medications used to prevent weak bones such as calcium and vitamin D supplements, phosphate binders, and the like.

In certain embodiments, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically DAM 116 of decision support engine 114, can fetch inputs 128 from user database 110 and compute a plurality of metrics 130 which can then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored according to an associated timestamp. The timestamp associated with information stored in historical records database 112 may identify, for example, when information related to a user has been obtained and/or updated.

Further, historical records database 112 may maintain time series data collected for users over a period of time, including for users who use continuous analyte monitoring system 104 and application 106. For example, analyte data for a user who has used continuous analyte monitoring system 104 and application 106 for a period of five years to manage the user's health may have time series analyte data associated with the user maintained over the five-year period.

Further, in certain embodiments, historical records database 112 may include data for one or more patients who are not users of continuous analyte monitoring system 104 and/or application 106. For example, historical records database 112 may include information (e.g., user profile(s)) related to one or more patients analyzed by, for example, a healthcare physician (or other known method), and not previously diagnosed with diabetes and/or kidney disease, as well as information (e.g., user profile(s)) related to one or more patients who were analyzed by, for example, a healthcare physician (or other known method) and were previously diagnosed with (varying types and stages of) diabetes and/or kidney disease. Data stored in historical records database 112 may be referred to herein as population data.

Data related to each patient stored in historical records database 112 may provide time series data collected over the disease lifetime of the patient, wherein the disease may be diabetes and/or kidney disease. For example, the data may include information about the patient prior to being diagnosed with kidney disease and information associated with the patient during the lifetime of the disease, including information related to each stage of kidney disease as it progressed and/or regressed in the patient, as well as information related to other diseases, such as hyperkalemia, hypokalemia, diabetes, hypertension, heart conditions and diseases, or similar diseases that are co-morbid in relation to kidney disease. Such information may indicate symptoms of the patient, physiological states of the patient, potassium levels of the patient, glucose levels of the patient, lactate levels of the patient, pyruvate levels of the patient, insulin levels of the patient, ketone levels of the patient, states/conditions of one or more organs of the patient, habits of the patient (e.g., activity levels, food consumption, etc.), medication prescribed, etc., throughout the lifetime of kidney disease.

In another example, the data may include information about the patient prior to being diagnosed with diabetes, hyperglycemia, hypoglycemia and information associated with the patient during the lifetime of the disease, including information related to diabetes as it progressed and/or regressed in the patient, as well as information related to other diseases, such as hyperglycemia, hypoglycemia, kidney disease, hypertension, heart conditions and diseases, or similar diseases that are co-morbid in relation to diabetes. Such information may indicate symptoms of the patient, physiological states of the patient, glucose levels of the patient, potassium levels of the patient, lactate levels of patient, insulin levels of the patients, states/conditions of one or more organs of the patient, habits of the patient (e.g., activity levels, food consumption, etc.), medication prescribed, medication adherence, etc., throughout the lifetime of the disease.

Although depicted as separate databases for conceptual clarity, in some embodiments, user database 110 and historical records database 112 may operate as a single database. That is, historical and current data related to users of continuous analyte monitoring system 104 and application 106, as well as historical data related to patients that were not previously users of continuous analyte monitoring system 104 and application 106, may be stored in a single database. The single database may be a storage server that operates in a public or private cloud.

As mentioned previously, decision support system 100 is configured to diagnose, stage, treat, and assess risks of diabetes, as well as predict the likelihood of experiencing atypical glucose trends and/or atypical glucose trends associated with kidney disease, for a user using continuous analyte monitoring system 104, including, at least, a continuous glucose sensor. For example, decision support engine 114 may be configured to collect information associated with a user in user profile 118 stored in user database 110, to perform analytics thereon to (1) automatically detect abnormal patterns associated with various glucose metrics, (2) assess the presence and severity of diabetes, (3) risk stratify patients to identify those patients with a high risk of diabetes, (4) identify risks (e.g., mortality risk, hyperglycemia risk, hypoglycemia risk, etc.) associated with a current diabetes diagnosis, (5) make patient-specific treatment decisions or recommendations for managing glucose homeostasis and kidney disease, and (6) provide information on the effect of an intervention (e.g., an effect of a lifestyle change of the patient, an effect of a surgical procedure, an effect of the patient taking new medication, an effect of the patient taking insulin, etc.). Further, in certain embodiments, each user's glucose metrics recorded over time may be analyzed to provide an indication of the improvement or the deterioration of the patient's diabetes. In certain embodiments, a user's glucose metrics may include glucose levels, glucose level rate(s) of change, glucose trend(s), mean glucose, glucose management indicator (GMI), glycemic variability, time in range (TIR), glucose clearance rate, etc.

In certain embodiments, decision support engine 114 may be used to collect information associated with a user in user profile 118 to perform analytics thereon for determining the probability of the presence and/or severity of diabetes for the user and providing one or more recommendations for treatment based on the determination. For example, decision support engine 114 may perform analytics on collected information associated with a user in user profile 118 to determine one or more glucose metrics and generate a diabetes diagnosis prediction based on the determined one or more glucose metrics for the user. In certain embodiments, decision support engine 114 may be used to collect information associated with a user in user profile 118 to perform analytics thereon for determining the likelihood of the user experiencing one or more atypical glucose trends associated with diabetes and kidney disease, and providing one or more recommendations for treatment based on the determination.

User profile 118 may be accessible to decision support engine 114 over one or more networks (not shown) for performing such analytics. In certain embodiments, decision support engine 114 is configured to provide real-time and/or non-real-time decision support around diabetes to the user and/or others, including but not limited, to healthcare providers (HCP), family members of the user, caregivers of the user, researchers, and/or other individuals, systems, and/or groups supporting care or learning from the data.

In certain embodiments, decision support engine 114 may utilize one or more trained machine learning models for (1) predicting the presence and/or severity of diabetes in a user with kidney disease (e.g., CKD), (2) predicting the presence/severity of diabetes in a user with unknown but suspected kidney disease, 3) predicting the presence/severity of kidney disease (e.g., CKD) in a user with pre-diabetes or “mild” type II, 4) predicting the presence/severity of diabetes in a user with known mild CKD but unknown diabetes status, 5) predicting presence/severity of diabetes and or CKD when both are of unknown status and (2) providing decision support to the user with diabetes and kidney disease based on information provided in the user's profile 118. In the illustrated embodiment of FIG. 1, decision support engine 114 may utilize trained machine learning model(s) provided by a training server system 140. Although depicted as a separate server for conceptual clarity, in some embodiments, training server system 140 and decision support engine 114 may operate as a single server. That is, the model may be trained and used by a single server, or may be trained by one or more servers and deployed for use on one or more other servers. In certain embodiments, the model may be trained on one or many virtual machines (VMs) running, at least partially, on one or many physical servers in relational and/or non-relational database formats.

Training server system 140 is configured to train the machine learning model(s) using training data, which may include data (e.g., from user profiles) associated with one or more patients (e.g., users or non-users of continuous analyte monitoring system 104 and/or application 106) previously diagnosed with (1) no diabetes and kidney disease, (2) no diabetes and varying stages of kidney disease, (3) varying stages of diabetes and no kidney disease, or (4) varying stages of diabetes and varying stages of kidney disease. The training data may be stored in historical records database 112 and may be accessible to training server system 140 over one or more networks (not shown) for training the machine learning model(s). The training data may also, in some cases, include user-specific data for a user over time.

The training data refers to a dataset that, for example, has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, may be a feature used in training the machine learning model. Such features may include the user's age; gender; various glucose metrics including glucose levels; change (e.g., delta) in glucose levels from a first timestamp to a second timestamp; glucose levels over time (e.g., glucose levels from two or more subsequent timestamps); mean glucose levels, change (e.g., delta) in mean glucose from a first series of timestamps (e.g., mean glucose from a first timestamp to one or more subsequent timestamps) to a second series of timestamps (e.g., mean glucose levels from a second timestamp to one or more subsequent timestamps); mean glucose levels over time (e.g., day to day, week to week, month to month, etc.) (e.g., mean glucose from two or more subsequent timestamps); mean glucose levels over the course of a first day compared to mean glucose levels over the course of a second day (e.g., mean glucose levels from the morning, afternoon, or evening, for example); mean glucose levels during event specific time ranges on a first day (e.g., morning, before going to sleep, during sleep, post-exercise, post-dialysis) compared to mean glucose levels during event specific time ranges on a second day; glucose management indicator (GMI); change (e.g., delta) in GMI from a first timestamp to a second timestamp; GMI over time (e.g., GMI from two or more subsequent timestamps); glycemic variability (e.g., standard deviation of mean glucose); change (e.g., delta) in glycemic variability from a first series of timestamps; (e.g., glycemic variability from a first timestamp to one or more subsequent time stamps) to a second series of timestamps (e.g., glycemic variability from a second timestamp to one or more subsequent timestamps); glycemic variability over time (e.g., glycemic variability from two or more subsequent timestamps); time in range (TIR) (e.g., glucose levels at, above, below, or between a threshold); change (e.g., delta) in TIR from a first timestamp to a second timestamp; TIR over time (e.g., TIR from two or more subsequent timestamps); glucose clearance rate; change (e.g., delta) in glucose clearance rate from a first time stamp to one or more subsequent timestamps; glucose clearance rate over time (e.g., blood and/or kidney glucose clearance rate from two or more subsequent timestamps; information associated with glucose metabolism, insulin activity/insulin insensitivity, change (e.g., delta) in a glucose metric (e.g., mean glucose, glucose management indicator (GMI), glycemic variability, time in range (TIR), glucose clearance rate, etc.) from a first series of timestamps (e.g., change in glucose levels from a first timestamp to one or more subsequent timestamps) to a second series of timestamps (e.g., change in glucose levels from a first timestamp to one or more subsequent timestamps); glucose metric over time (e.g., glucose metric from two or more subsequent timestamps); change (e.g., delta) in kidney disease stage or severity from a first timestamp to a second timestamp; change (e.g., delta) in diabetes stage or severity from a first timestamp to a second timestamp; the derivative of the determined linear system of a glucose metric at a specific timestamp, or specific series of timestamps, and/or the difference in derivatives to determine rates of change in the slope of the increase or decrease in a glucose metric; the derivative of the measured linear system of glucose level at one or more specific timestamps and/or the difference in derivatives to determine rates of change in the slope of the increase or decrease in glucose levels; the derivative of the determined linear system of mean glucose at a specific timestamp, or a specific series of timestamps, and/or the difference in derivatives to determine rates of change in the slope of the increase or decrease in mean glucose; the derivative of the determined linear system of GMI at a specific timestamp, or a specific series of timestamps, and/or the difference in derivatives to determine rates of change in the slope of the increase or decrease in GMI; the derivative of the determined linear system of glycemic variability at a specific timestamp, or a specific series of timestamps, and/or the difference in derivatives to determine rates of change in the slope of the increase or decrease in glycemic variability; the derivative of the determined linear system of TIR at a specific timestamp, or a specific series of timestamps, and/or the difference in derivatives to determine rates of change in the slope of the increase or decrease in TIR; the derivative of the determined linear system of glucose clearance rate at a specific timestamp, or a specific series of timestamps, and/or the difference in derivatives to determine rates of change in the slope of the increase or decrease in glucose clearance rate, etc.

Other features may include the user's potassium levels; change (e.g., delta) in potassium levels from a first timestamp to a second timestamp; potassium levels over time (e.g., potassium levels from two or more subsequent timestamps); the derivative of the measured linear system of potassium measurement at one or more specific timestamps and/or the difference in derivatives to determine rates of change in the slope of the increase or decrease in potassium levels; potassium levels over time (e.g., potassium levels from two or more subsequent timestamps); mean potassium levels, change (e.g., delta) in mean potassium from a first series of timestamps (e.g., mean potassium from a first timestamp to one or more subsequent timestamps) to a second series of timestamps (e.g., mean potassium levels from a second timestamp to one or more subsequent timestamps); mean potassium levels over time (e.g., mean potassium from two or more subsequent timestamps); change (e.g., delta) in potassium variability from a first series of timestamps; (e.g., potassium variability from a first timestamp to one or more subsequent time stamps) to a second series of timestamps (e.g., potassium variability from a second timestamp to one or more subsequent timestamps); potassium variability over time (e.g., potassium variability from two or more subsequent timestamps); time in range (TIR) (e.g., potassium levels at, above, below, or between a threshold); change (e.g., delta) in TIR from a first timestamp to a second timestamp; TIR over time (e.g., TIR from two or more subsequent timestamps); potassium clearance rate; change (e.g., delta) in potassium clearance rate from a first time stamp to one or more subsequent timestamps; potassium clearance rate over time (e.g., blood and/or kidney potassium clearance rate from two or more subsequent timestamps; change (e.g., delta) in a potassium metric (e.g., mean potassium, potassium variability, time in range (TIR), potassium clearance rate, etc.) from a first series of timestamps (e.g., change in potassium levels from a first timestamp to one or more subsequent timestamps) to a second series of timestamps (e.g., change in potassium levels from a first timestamp to one or more subsequent timestamps); potassium metric over time (e.g., potassium metric from two or more subsequent timestamps).

Other features may include the user's lactate level; change (e.g., delta) in lactate levels from a first timestamp to a second timestamp; lactate levels over time (e.g., lactate levels from two or more subsequent timestamps); the derivative of the measured linear system of lactate level at one or more specific timestamps and/or the difference in derivatives to determine rates of change in the slope of the increase or decrease in lactate levels; lactate levels over time (e.g., lactate levels from two or more subsequent timestamps); mean lactate levels, change (e.g., delta) in mean lactate from a first series of timestamps (e.g., mean lactate from a first timestamp to one or more subsequent timestamps) to a second series of timestamps (e.g., mean lactate levels from a second timestamp to one or more subsequent timestamps); mean lactate levels over time (e.g., mean lactate from two or more subsequent timestamps); lactate variability (e.g., standard deviation of mean lactate); change (e.g., delta) in lactate variability from a first series of timestamps; (e.g., lactate variability from a first timestamp to one or more subsequent time stamps) to a second series of timestamps (e.g., lactate variability from a second timestamp to one or more subsequent timestamps); lactate variability over time (e.g., lactate variability from two or more subsequent timestamps); time in range (TIR) (e.g., lactate levels at, above, below, or between a threshold); change (e.g., delta) in TIR from a first timestamp to a second timestamp; TIR over time (e.g., TIR from two or more subsequent timestamps); lactate clearance rate; change (e.g., delta) in lactate clearance rate from a first time stamp to one or more subsequent timestamps; lactate clearance rate over time (e.g., blood and/or kidney lactate clearance rate from two or more subsequent timestamps; change (e.g., delta) in a lactate metric (e.g., mean lactate, lactate management indicator (GMI), lactate variability, time in range (TIR), lactate clearance rate, etc.) from a first series of timestamps (e.g., change in lactate levels from a first timestamp to one or more subsequent timestamps) to a second series of timestamps (e.g., change in lactate levels from a first timestamp to one or more subsequent timestamps); lactate metric over time (e.g., lactate metric from two or more subsequent timestamps).

Other features may include the user's ketone level; change (e.g., delta) in ketone levels from a first timestamp to a second timestamp; ketone levels over time (e.g., ketone levels from two or more subsequent timestamps); the derivative of the measured linear system of ketone level at one or more specific timestamps and/or the difference in derivatives to determine rates of change in the slope of the increase or decrease in ketone levels; pyruvate level; change (e.g., delta) in pyruvate levels from a first timestamp to a second timestamp; pyruvate levels over time (e.g., pyruvate levels from two or more subsequent timestamps); the derivative of the measured linear system of pyruvate level at one or more specific timestamps and/or the difference in derivatives to determine rates of change in the slope of the increase or decrease in pyruvate levels; insulin level; change (e.g., delta) in insulin levels from a first timestamp to a second timestamp; insulin levels over time (e.g., insulin levels from two or more subsequent timestamps); the derivative of the measured linear system of insulin level at one or more specific timestamps and/or the difference in derivatives to determine rates of change in the slope of the increase or decrease in insulin levels.

In addition, other features may include non-analyte data; change (e.g., delta) in non-analyte data from a first timestamp to a second timestamp; non-analyte data over time (e.g., non-analyte data from two or more subsequent timestamps); the derivative of the measured linear system of non-analyte data at one or more specific timestamps and/or the difference in derivatives to determine rates of change in the slope of the increase or decrease in non-analyte data; etc. In addition, the data record is labeled with an indication as to a diabetes diagnosis, an assigned disease severity and/or type, an identified risk of diabetes, kidney disease diagnosis, assigned disease severity and/or type, an identified risk of other diseases, medication and/or therapeutics initiated, discontinued, and/or administered, including the type, dose and associated timestamps, insulin initiated, discontinued and/or administered, including the type, dose and associated timestamp, food consumed, sleep, stress, heart rate, blood pressure, initiation and/or discontinuation of exercise, etc., associated with a patient of the user profile.

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, in certain embodiments, the model(s) may be iteratively refined to generate accurate predictions associated with diabetes risk, presence, progression, improvement, and severity in a patient. Further, in certain other embodiments, by iteratively processing each data record corresponding to each historical patient, in certain embodiments, the model(s) may be iteratively refined to generate accurate predictions of the risk and/or presence of one or more symptoms associated with reduced glucose homeostasis.

As illustrated in FIG. 1, training server system 140 deploys these trained model(s) to decision support engine 114 for use during runtime. For example, decision support engine 114 may obtain user profile 118 associated with a user, use information in user profile 118 as input into the trained model(s), and output a prediction. The prediction may be indicative of the presence and/or severity of diabetes for the user or indicative of the presence or a risk of the user experiencing at least one atypical glucose trend in real-time or within a certain time (e.g., shown as output 144 in FIG. 1). Output 144 generated by decision support engine 114 may also provide one or more decision support recommendations for treatment based on the predictions. Output 144 may be provided to the user (e.g., through application 106), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

In certain embodiments, the user's own data is used to personalize the one or more models that are initially trained based on population data. For example, a model (e.g., trained using population data) may be deployed for use by decision support engine 114 to predict the presence or a risk of a specific user experiencing an atypical glucose trend in real-time. After making a prediction using the model, decision support engine 114 may be configured to obtain the user's actual glucose trend and compute a loss between the prediction and the actual glucose trend, which can be used for retraining the model. Accordingly, the model may continue to be retrained and personalized using the computed loss between the prediction and the actual glucose trend as input into the model to personalize the model for the user. In another example, a model (e.g., trained using population data) may be deployed for use by decision support engine 114 to predict the effect of treatment (e.g., medication administration, food consumption, etc.) on a specific user experiencing an atypical glucose trend in real-time. After making a prediction using the model, decision support engine 114 may be configured to obtain the user's actual glucose trend and compute a loss between the prediction and the actual glucose trend, which can be used for retraining the model. Accordingly, the model may continue to be retrained and personalized using the computed loss between the prediction and the actual glucose trend as input into the model to personalize the model for the user.

In certain embodiments, output 144 generated by decision support engine 114 may be stored in user profile 118. In certain embodiments, output 144 may be a prediction as to the presence, type, and/or severity of diabetes in a user. In certain embodiments, output 144 may be patient-specific treatment decisions or recommendations for managing diabetes. In certain embodiments, output 144 may be a prediction as to the risk of a user having an atypical glucose trend. In certain embodiments, output 144 may be an adverse glycemic event prediction. In certain embodiments, output 144 may be patient-specific treatment decisions or recommendations for glycemic homeostasis. Output 144 stored in user profile 118 may be continuously updated by decision support engine 114. Accordingly, previous diagnoses and/or glucose metrics of the user, originally stored as outputs 144 in user profile 118 in user database 110 and then passed to historical records database 112, may provide an indication of the progression of diabetes in a user over time, as well as provide an indication as to the effectiveness of different treatments (e.g., medications) recommended to a user to maintain glucose homeostasis.

In certain embodiments, a user's own historical data may be used to provide decision support and insight around the user's glucose homeostasis and/or disease. For example, a user's historical data may be used by an algorithm as a baseline to indicate improvements or deterioration in the user's glucose homeostasis. As an illustrative example, a user's data from two weeks prior may be used as a baseline that can be compared with the user's current data to identify whether the user's glucose homeostasis has improved or deteriorated. In certain embodiments, the user's own historical data may be used by training server system 140 to train a personalized model that may further be able to predict or project out the user's glucose level or the user's glucose homeostasis based on the user's recent pattern of data (e.g., glucose metrics, exercise data, food consumption data, medication data, etc.).

In certain embodiments, the model may be trained to provide lifestyle recommendations, exercise recommendations, diet recommendations, medication recommendations, medical recommendations, and other types of decision support recommendations to help the user manage glucose homeostasis based on the user's historical data, including how different types of medication, food, and treatment (e.g., such as insulin) have impacted the user's glucose homeostasis in the past. In certain embodiments, the model may be trained to detect the underlying cause of certain improvements or deteriorations in the patient's glucose homeostasis. For example, application 106 may display a user interface with a graph that shows the patient's glucose metrics or a score thereof with trend lines and indicate, e.g., retrospectively, what caused the glucose homeostasis to suffer at certain points in time (e.g., excess glucose intake, insulin dosage, declining kidney function, etc.).

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise a sensor for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 202 provides a data stream indicative of the concentration of one or more analytes in the user. The data stream may include raw data signals, which are then converted into a calibrated and/or filtered data stream used to provide estimated analyte value(s) to the user.

In certain embodiments, continuous analyte sensor 202 may be a multi-analyte sensor, configured to continuously measure multiple analytes in a user's body. For example, in certain embodiments, the continuous multi-analyte sensor 202 may be a single multi-analyte sensor configured to measure potassium, glucose, lactate, ketones, pyruvate, and insulin in the user's body.

In certain embodiments, one or more multi-analyte sensors may be used in combination with one or more single analyte sensors. As an illustrative example, a multi-analyte sensor may be configured to continuously measure potassium and glucose and may, in some cases, be used in combination with an analyte sensor configured to measure only lactate levels. Information from each of the multi-analyte sensor(s) and single analyte sensor(s) may be combined to provide diabetes decision support using methods described herein.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enables measurement of levels of analyte(s) via a continuous analyte sensor(s) 202. For example, sensor electronics module 204 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 can include a display such as a touchscreen display 212, 222, 232, /or 242 for displaying sensor data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or receiving user inputs. Display devices 210, 220, 230, and 240 may be examples of display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data. In certain embodiments, the type of alarms customized for each particular display device, the number of alarms customized for each particular display device, the timing of alarms customized for each particular display device, and/or the threshold levels configured for each of the alarms (e.g., for triggering) are based on output 144 (e.g., as mentioned, output 144 may be indicative of the current health of a user, the state of a user's glucose, and/or current treatment recommended to a user) stored in user profile 118 for each user. In certain embodiments, one or more of the display devices may each have a user interface that may include a variety of interfaces, such a liquid crystal display (LCD) for presenting a UI features, a vibrator, an audio transducer (e.g., speaker), a backlight (not shown), and/or the like. The components that comprise such a user interface may provide controls to interact with the user (e.g., the host). One or more UI features may allow, for example, toggle, menu selection, option selection, status selection, yes/no response to on-screen questions, a “turn off” function (e.g., for an alarm), an “acknowledged” function (e.g., for an alarm), a reset, and/or the like. The UI features may also provide the user with, for example, visual data output. The audio transducer (e.g., speaker) may provide audible signals in response to triggering of certain alerts, such as present and/or predicted conditions. In some example implementations, audible signals may be differentiated by tone, volume, duty cycle, pattern, duration, and/or the like. In some example implementations, the audible signal may be configured to be silenced (e.g., acknowledged or turned off) by pressing one or more buttons.

As mentioned, sensor electronics module 204 may be in communication with a medical device 208. Medical device 208 may be a passive device in some example embodiments of the disclosure. For example, medical device 208 may be an insulin pump for administering insulin to a user. For a variety of reasons, it may be desirable for such an insulin pump to receive and track glucose, potassium, lactate, insulin, ketone, and/or pyruvate values transmitted from continuous analyte monitoring system 104, where continuous analyte sensor 202 is configured to measure glucose, potassium, lactate, insulin, ketone and/or pyruvate.

Further, as mentioned, sensor electronics module 204 may also be in communication with other non-analyte sensors 206. Non-analyte sensors 206 may include, but are not limited to, an altimeter sensor, an accelerometer sensor, a temperature sensor, a respiration rate sensor, a sweat sensor, etc. Non-analyte sensors 206 may also include monitors such as heart rate monitors, ECG monitors, blood pressure monitors, pulse oximeters, caloric intake, and medicament delivery devices. One or more of these non-analyte sensors 206 may provide data to decision support engine 114 described further below. In some aspects, a user may manually provide some of the data for processing by training server system 140 and/or decision support engine 114 of FIG. 1.

In certain embodiments, the non-analyte sensors 206 may be combined in any other configuration, such as, for example, combined with one or more continuous analyte sensors 202. As an illustrative example, a non-analyte sensor, e.g., a heart rate sensor, may be combined with a continuous analyte sensor 202 configured to measure glucose to form a glucose/heart rate sensor used to transmit sensor data to sensor electronics module 204 using common communication circuitry. As another illustrative example, a non-analyte sensor, e.g., a heart rate sensor and/or an ECG sensor, may be combined with a multi-analyte sensor 202 configured to measure glucose and potassium to form glucose/potassium/heart rate sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and/or Bluetooth may also be used among devices depicted in diagram 200 of FIG. 2.

FIG. 3 illustrates example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and decision support engine 114 including DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors/monitors, other applications executing on display device 107, EMRs, etc.). As mentioned previously, in certain embodiments, inputs 128 may be processed by DAM 116 and/or decision support engine 114 to output metrics 130. Inputs and metrics 130 may be used by decision support engine 114 to provide decision support to the user. For example, inputs 128 and metrics 130 may be used by training server system 140 to train and deploy one or more machine learning models for use by decision support engine 114 for providing decision support around diabetes for patients with kidney disease.

In certain embodiments, starting with inputs 128, food consumption information may include information about one or more of meals, snacks, and/or beverages, such as one or more of the size, content (milligrams (mg) of potassium, glucose, lactate, carbohydrate, fat, protein, etc.), sequence of consumption, and time of consumption. In certain embodiments, food consumption may be provided by a user through manual entry, by providing a photograph through an application that is configured to recognize food types and quantities (e.g., potassium and glucose/carbohydrate content of foods), and/or by scanning a bar code or menu. In various examples, meal size may be manually entered as one or more of calories, quantity (e.g., “three cookies”), menu items (e.g., “Royale with Cheese”), and/or food exchanges (e.g., 1 fruit, 1 dairy). In some examples, meal information may be received via a convenient user interface provided by application 106.

In certain embodiments, food consumption information entered by a user may relate to glucose consumed by the user. Glucose for consumption may include any natural or designed food or beverage that contains glucose, dextrose or carbohydrate, such as glucose tablet, a banana, or bread, for example.

In certain embodiments, exercise information is also provided as an input. Exercise information may be any information surrounding activities, such as activities requiring physical exertion by the user. For example, exercise information may range from information related to low intensity (e.g., walking a few steps) and high intensity (e.g., five-mile run) physical exertion. In certain embodiments, exercise information may be provided, for example, by an accelerometer sensor on a wearable device such as a watch, fitness tracker, and/or patch. In certain embodiments, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and/or the level of strenuous exertion being used during the exercise over a specific period. This data may be used to train the system 100 to learn about the user's exercise patterns to reduce the need for confirmation questions as time progresses.

In certain embodiments, user statistics, such as one or more of age, height, weight, BMI, body composition (e.g., % body fat), stature, build, or other information may also be provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment/medication information is also provided as an input. Medication information may include information about the type, dosage, and/or timing of when one or more medications are to be taken by the user. As mentioned herein, the medication information may include information about one or more glycemic controlling medication, one or more drugs known to damage the kidney, one or more drugs known to control the complications of kidney disease that are prescribed to the user, and/or one or more medications for treating one or more symptoms of kidney disease, hyperkalemia, hypokalemia, diabetes, and/or other conditions and diseases the user may have. Treatment information may include information regarding different lifestyle habits, surgical procedures, and/or other non-invasive procedures recommended by the user's physician. For example, the user's physician may recommend a user increase/decrease their glucose intake, exercise for a minimum of thirty minutes a day, and/or increase an insulin dosage or other medication to maintain, and/or improve, kidney health, glucose homeostasis, general health, etc. In certain embodiments, treatment/medication information may be provided through manual user input.

In certain embodiments, analyte sensor data may also be provided as input, for example, through continuous analyte monitoring system 104. In certain embodiments, analyte sensor data may include glucose data (e.g., a user's glucose values) measured by at least a continuous glucose sensor (or multi-analyte sensor configured to measure at least glucose) that is a part of continuous analyte monitoring system 104. In certain embodiments, analyte sensor data may include potassium data measured by at least a potassium sensor (or multi-analyte sensor configured to measure at least potassium) that is a part of continuous analyte monitoring system 104. In certain embodiments, analyte sensor data may include lactate data measured by at least a lactate sensor (or multi-analyte sensor configured to measure at least lactate) that is a part of continuous analyte monitoring system 104. In certain embodiments, analyte sensor data may include insulin data measured by at least an insulin sensor (or multi-analyte sensor configured to measure at least insulin) that is a part of continuous analyte monitoring system 104. In certain embodiments, analyte sensor data may include pyruvate data measured by at least a pyruvate sensor (or multi-analyte sensor configured to measure at least pyruvate) that is a part of continuous analyte monitoring system 104. In certain embodiments, analyte sensor data may include ketone data measured by at least a ketone sensor (or multi-analyte sensor configured to measure at least ketone) that may be a part of continuous analyte monitoring system 104.

In certain embodiments, input may also be received from one or more non-analyte sensors, such as non-analyte sensors 206 described with respect to FIG. 2. Input from such non-analyte sensors 206 may include information related to a heart rate, heart rate variability (e.g., the variance in time between the beats of the heart), ECG data, a respiration rate, oxygen saturation, a blood pressure, or a body temperature (e.g., to detect illness, physical activity, etc.) of a user. In certain embodiments, electromagnetic sensors may also detect low-power radio frequency (RF) fields emitted from objects or tools touching or near the object, which may provide information about user activity or location.

In certain embodiments, input received from non-analyte sensors may include input relating to a user's insulin delivery. In particular, input related to the user's insulin delivery may be received, via a wireless connection on a smart insulin pen, via user input, and/or from an insulin pump. Insulin delivery information may include one or more of insulin volume, time of delivery, etc. Other parameters, such as insulin action time, insulin activity rate or duration of insulin action, may also be received as inputs.

In certain embodiments, time may also be provided as an input, such as time of day or time from a real-time clock. For example, in certain embodiments, input analyte data may be timestamped to indicate a date and time when the analyte measurement was taken for the user.

User input of any of the above-mentioned inputs 128 may be through a user interface, such a user interface of display device 107 of FIG. 1.

As described above, in certain embodiments, DAM 116 and/or decision support engine (e.g., using one or more trained models) determines or computes the user's metrics 130 based on inputs 128. An example list of metrics 130 is shown in FIG. 3.

In certain embodiments, glucose levels may be determined from sensor data (e.g., glucose levels obtained from a continuous glucose sensor of continuous analyte monitoring system 104). For example, glucose levels refer to time-stamped glucose levels or values that are continuously generated and stored over time. In certain embodiments, a glucose metric refers to one or more time-stamped glucose levels or values. A glucose metric may refer to, include, or indicate a change between one or more time-stamped glucose levels or values. For example, a glucose metric may include or indicate the difference between a glucose level at a first timestamp and a glucose level at a subsequent time stamp. A glucose metric may further include or indicate a glucose rate of change which refers to a rate that indicates the change of one or more time-stamped glucose levels or values in relation to one or more other time-stamped glucose levels or values. For example, a glucose metric may include or indicate the derivative of the change between a glucose level at a first timestamp and the glucose level at one or more subsequent timestamps. A glucose metric may be the mean glucose level at a specific time of day or following an event, such as meal or physical activity, for example. A specific time of day may comprise a range of times when multiple analyte measurements are taken across one or more time periods (e.g., time ranges). The mean glucose level at a specific time of day may be compared to subsequent days to determine changes in glucose levels during specific time periods over time, which may demonstrate disease progression. Additionally, a glucose metric may include the frequency of deviation from an expected day time mean glucose threshold.

In certain embodiments, a glucose metric may include glucose levels, glucose rate of change, mean glucose, GMI, glycemic variability, TIR, and/or glucose clearance rate. Further, in certain embodiments, a glucose metric may include a change in one or more glucose metrics, such as an absolute change, a rate of change, etc. In certain embodiments, DAM 116 may continuously calculate a glucose metric, time-stamp the calculated glucose metric, and store the corresponding information in the user's profile 118.

In certain other embodiments, DAM 116 may exclude glucose levels measured over a period of time when an external condition exists that would affect the user's glucose homeostasis (e.g., where the user is, at least for a subset of the period of time, engaging in exercise, consuming glucose, or other similar situations). In such embodiments, DAM 116 may, in some examples, first identify which measured glucose values are not to be used for calculating the glucose metric by identifying which glucose values have been affected by an external event, such as the consumption of food, exercise, medication, or other perturbation that would disrupt the capture of a glucose metric. DAM 116 may then exclude such measurements when calculating the glucose metrics of the user.

In certain embodiments, an absolute maximum glucose level may be determined from sensor data (e.g., glucose levels obtained from a continuous glucose sensor of continuous analyte monitoring system 104), health/sickness metrics (e.g., described in more detail below), and/or disease stage metrics (e.g., described in more detail below). The absolute maximum glucose level represents a user's maximum glucose level determined to be unsafe over a period of time (e.g., hourly, weekly, daily, etc.). In certain embodiments, the absolute maximum glucose level may be consistent across all users (e.g., set to 200 mg/dL based on current medical guidelines). In certain other embodiments, each patient may have a different absolute maximum glucose level. For example, an absolute maximum glucose level may be lower for a user diagnosed with stage 1 CKD (e.g., normal or high GFR (GFR>90 mL/min)) than a user diagnosed with stage 5 end stage CKD (e.g., GFR<15 mL/min). In certain embodiments, the absolute maximum glucose level per patient may change over time. For example, a user may be initially assigned an absolute maximum glucose level based on clinical input. This assigned absolute maximum glucose level may be adjusted over time based on other sensor data, disease stages, comorbidities, etc., for the user.

For example, a user's absolute maximum glucose level may vary over time as a user's kidney function, kidney disease, and/or one or more other diseases progress and/or improve. In certain embodiments, a first absolute maximum glucose level may be determined for periods of time where no external conditions exist that would affect the glucose level, and a second absolute maximum glucose level may be determined for periods of time where external conditions do exist that would affect the glucose level (e.g., during periods of time when the user is consuming glucose, exercising, taking medication that affects glucose levels, etc.).

In certain embodiments, an absolute minimum glucose level may be determined from sensor data (e.g., glucose levels obtained from a continuous glucose sensor of continuous analyte monitoring system 104), health/sickness metrics (e.g., described in more detail below), and/or disease stage metrics (e.g., described in more detail below). The absolute minimum glucose level represents a user's minimum glucose level determined to be unsafe over a period of time (e.g., hourly, weekly, daily, etc.). In certain embodiments, the absolute minimum glucose level may be consistent across all users (e.g., set based on current medical guidelines). In certain other embodiments, each user may have a different absolute minimum glucose level. For example, an absolute minimum glucose level may be lower for a user diagnosed with stage 1 CKD (e.g., normal or high GFR (GFR>90 mL/min)) than a user diagnosed with stage 5 end stage CKD (e.g., GFR<15 mL/min). In certain embodiments, the absolute minimum glucose level per patient may change over time. For example, a user may be initially assigned an absolute minimum glucose level based on clinical input. This assigned absolute minimum glucose level may be adjusted over time based on other sensor data, disease stages, comorbidities, etc., for the user.

For example, a user's absolute minimum glucose level may vary over time as a user's kidney function, kidney disease, diabetes, and/or one or more other diseases progress and/or improve. In certain embodiments, a first absolute minimum glucose level may be determined for periods of time where no external conditions exist that would affect the glucose level, and a second absolute minimum glucose level may be determined for periods of time where external conditions do exist that would affect the glucose level (e.g., during periods of time when the user is consuming glucose, exercising, taking medication that affects glucose levels, etc.). In certain embodiments, an absolute minimum glucose level may be determined for periods of time when the user is asleep.

In certain embodiments, glucose level rates of change may be determined from sensor data (e.g., glucose levels obtained from a continuous glucose sensor of continuous analyte monitoring system 104 over time). For example, a glucose level rate of change refers to a rate that indicates how one or more time-stamped glucose levels or values change in relation to one or more other time-stamped glucose levels or values. Glucose level rates of change may be determined over one or more seconds, minutes, hours, days, etc.

In certain embodiments, determined glucose level rates of change may be marked as “increasing rapidly” or “decreasing rapidly”. As used herein, “rapidly” may describe glucose level rates of change that are clinically significant and pointing towards a trend of the glucose level of the patient likely breaching the absolute maximum glucose level or the absolute minimum glucose level within a defined next period of time. In other words, a predictive trend (e.g., produced by decision support engine 114 using one or more trained models) may, in some cases, indicate that a patient is likely to hit, for example, the absolute maximum glucose level within a specified time period (e.g., one or two hours) based on the determined glucose level rate of change. Accordingly, such a glucose level rate of change may be marked as “increasing rapidly”. Similarly, a predictive trend (e.g., produced by decision support engine 114 using one or more trained models) may, in some cases, indicate that a patient is likely to hit the absolute minimum glucose level within a specified time period (e.g., one or two hours) based on the glucose level rate of change determined. Accordingly, such a glucose level rate of change may be marked as “decreasing rapidly”.

In certain embodiments, glucose metric rates of change may be determined from glucose metrics determined for a user over time. For example, a glucose metric rate of change refers to a rate that indicates how one or more time-stamped glucose metrics for a user change in relation to one or more other time-stamped glucose metrics for the same user. Glucose metric rates of change may be determined over one or more seconds, minutes, hours, days, etc.

In certain embodiments, a glucose clearance rate may be determined from sensor data (e.g., glucose levels obtained from a continuous glucose sensor of continuous analyte monitoring system 104) following the consumption of a known, or estimated, amount of glucose. Glucose clearance rates analyzed over time may be indicative of glucose homeostasis. In particular, the slope of a curve of glucose clearance during a first time period (e.g., after consuming a known amount of glucose) compared to the slope of a curve of glucose clearance during a second time period (e.g., after consuming the same amount of glucose) may be indicative of a kidney's ability to function, and more particularly, to maintain glucose homeostasis (e.g., a glucose clearance rate may be slower when a user's kidney is impaired than when a user's kidney is healthy).

In certain embodiments, the glucose clearance rate may be determined by calculating a slope between an initial high glucose value (e.g., highest glucose level during a period of 20-30 minutes after the consumption of glucose) and a subsequent low glucose value. The low glucose value (GL) may be determined based on a user's initial high glucose value (GH) and baseline glucose value (GB) before the consumption of glucose. In certain embodiments, GL can be a glucose value between GH and GB, e.g., GL=GB+K*(GH−GB)/2, where K can be a percentage representing how much a user's glucose level returned to the user's baseline value. When K equals zero, the low glucose value equals the baseline glucose value. When K equals 0.5, the low glucose value equals the mean glucose value between the initial glucose value and the baseline glucose value. In certain embodiments, the glucose clearance rate may be determined over one or more periods of time after the consumption of glucose. The glucose clearance rate may be calculated for each time period to represent the dynamics of glucose clearance rate after the consumption of glucose. These glucose clearance rates calculated over time may be time-stamped and stored in the user's profile 118. Certain metrics may be derived from the time-stamped glucose clearance rates, such as mean, median, standard deviation, percentile, etc. In certain embodiments, a user with CKD may have impaired kidney function to metabolize insulin and the time passed from the initial high glucose value to the low glucose value may be indicative of a kidney's ability to function. The time passed from the initial high glucose value to the low glucose value and the glucose clearance rates may be time-stamped and store in the user's profile 118.

In certain embodiments, glucose trends may be determined based on glucose levels over certain periods of time. In certain embodiments, glucose trends may be determined based on glucose metrics over certain periods of time. In certain embodiments, glucose trends may be determined based on absolute glucose level minimums over certain periods of time. In certain embodiments, glucose trends may be determined based on absolute maximum glucose levels over certain periods of time. In certain embodiments, glucose trends may be determined based on glucose level rates of change over certain periods of time. In certain embodiments, glucose trends may be determined based on rates of change of glucose metrics (e.g., glucose levels, mean glucose, GMI, glycemic variability, TIR, glucose clearance, etc.) over certain periods of time. In certain embodiments, glucose trends may be determined based on calculated glucose clearance rates over certain periods of time.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

In certain embodiments, insulin on board may be determined using non-analyte sensor data input (e.g., insulin delivery information) and/or known or learned (e.g., from user data) insulin time action profiles, which may account for both basal metabolic rate (e.g., uptake of insulin to maintain operation of the body) and insulin usage driven by activity or food consumption.

In certain embodiments, potassium levels may be determined from sensor data (e.g., potassium measurements obtained from continuous analyte monitoring system 104).

In certain embodiments, an absolute maximum potassium level may be determined from sensor data (e.g., potassium measurements obtained from a continuous potassium sensor of continuous analyte monitoring system 104), health/sickness metrics (e.g., described in more detail below), and/or disease stage metrics (e.g., described in more detail below). The absolute maximum potassium level represents a user's maximum potassium level determined to be unsafe over a period of time (e.g., hourly, weekly, daily, etc.). Each user may have a different absolute maximum potassium level. A user's absolute maximum potassium level may vary over time as a user's kidney function, kidney disease, and/or one or more other diseases progress and/or improve.

In certain embodiments, potassium level rates of change may be determined from sensor data (e.g., potassium measurements obtained from a potassium sensor of continuous analyte monitoring system 104 over time). For example, a potassium level rate of change refers to a rate that describes how one or more time-stamped potassium measurements or values change in relation to one or more other time-stamped potassium measurements or values. Potassium level rates of change may be determined over one or more seconds, minutes, hours, days, etc. In certain embodiments, average potassium levels may be calculated for determining rates of change of the calculated average potassium levels of the user.

In certain embodiments, potassium trends may be determined based on potassium levels over certain periods of time. In certain embodiments, information about potassium time in range (TIR) may also be determined based on potassium levels over time.

In certain embodiments, lactate levels may be determined from sensor data (e.g., lactate measurements obtained from continuous analyte monitoring system 104). In certain embodiments, lactate trends may be determined based on lactate levels over certain periods of time. In certain embodiments, information about lactate time in range (TIR) may also be determined based on lactate levels over time.

In certain embodiments, pyruvate levels may be determined from sensor data (e.g., pyruvate measurements obtained from continuous analyte monitoring system 104). In certain embodiments, pyruvate trends may be determined based on pyruvate levels over certain periods of time. In certain embodiments, information about pyruvate time in range (TIR) may also be determined based on pyruvate levels over time. In certain embodiments, pyruvate measurements may assist in understanding the rate of glucose generation and/or consumption.

In certain embodiments, ketone levels may be determined from sensor data (e.g., ketone measurements obtained from continuous analyte monitoring system 104). In certain embodiments, ketones trends may be determined based on ketones levels over certain periods of time. In certain embodiments, information about ketone time in range (TIR) may also be determined based on ketone levels over time.

In certain embodiments, health and sickness metrics may be determined, for example, based on one or more of user input (e.g., pregnancy information or known sickness or disease information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, disease stage metrics, such as for kidney disease, may be determined, for example, based on one or more of user input or output provided by decision support engine 114 illustrated in FIG. 1. In certain embodiments, example disease stages for kidney disease, may include AKI, stage 1 CKD with normal or high GFR (e.g., GFR>90 mL/min), stage 2 mild CKD (e.g., GFR=60-89 mL/min), stage 3A moderate CKD (e.g., GFR=45-59 mL/min), stage 3B moderate CKD (e.g., GFR=30-44 mL/min), stage 4 severe CKD (e.g., GFR=15-29 mL/min), and stage 5 end stage CKD (e.g., GFR<15 mL/min). In certain embodiments, example disease stages may be represented as a GFR value/range, severity score, and the like.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type (e.g., macronutrient and micronutrient content (e.g., glucose and potassium)), quantity, and/or sequence (e.g., which food/beverage was eaten first).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule or a recommended diet, the closer their meal habit metric will be to 1, in the example.

In certain embodiments, medication adherence is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage). In certain embodiments, medication adherence of a user may be determined in a clinical trial where medication consumption and timing of such medication consumption is monitored, through user input, and/or based on analyte data received from analyte monitoring system 104.

In certain embodiments, the activity level metric may indicate the user's level of activity. In certain embodiments, the activity level metric be determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, user input, etc. In certain embodiments, the activity level may be expressed as a step rate of the user. Activity level metrics may be time-stamped so that they can be correlated with the user's analyte levels at the same time.

In certain embodiments, exercise regimen metrics may indicate one or more of what type of activities the user engages in, the corresponding intensity of such activities, frequency the user engages in such activities, etc. In certain embodiments, exercise regimen metrics may be calculated based on one or more of non-analyte sensor data input (e.g., non-analyte sensor data input from an accelerometer, a heart rate monitor, an ECG sensor (e.g., for monitoring heart function, QRS complex, abnormalities, premature ventricular contractions (PVCs), premature atrial complex (PAC), etc.), temperature sensor, a respiration rate sensor, etc.), calendar input, user input, etc.

In certain embodiments, body temperature metrics may be calculated by DAM 116 based on inputs 128, and more specifically, non-analyte sensor data from a temperature sensor. In certain embodiments, heart rate metrics (e.g., including heart rate and heart rate variability) may be calculated by DAM 116 based on inputs 128, and more specifically, non-analyte sensor data from a heart rate sensor and/or ECG sensor. In certain embodiments, respiratory metrics may be calculated by DAM 113 based on inputs 128, and more specifically, non-analyte sensor data from a respiratory rate sensor.

### Example Methods and Systems for Providing Decision Support Around Diabetes and Kidney Disease

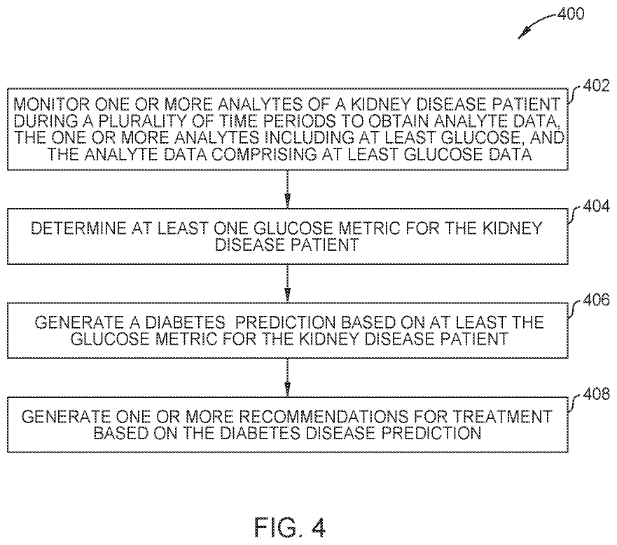

FIG. 4 is a flow diagram illustrating an example method 400 for providing decision support using a continuous analyte monitoring system including, at least, a continuous glucose sensor, in accordance with certain example aspects of the present disclosure. For example, method 400 may be performed to provide decision support to a user, using a continuous analyte monitoring system 104 including, at least, a continuous glucose sensor 202, as illustrated in FIGS. 1 and 2.

Method 400 may be performed by decision support system 100 to collect/generate data such as inputs 128 and metrics 130, including for example, analyte data, patient information, and non-analyte sensor data mentioned above, to (1) automatically detect patterns associated with glucose metrics, (2) assess the presence and severity of diabetes, (3) risk stratify patients to identify those patients with a high risk of diabetes, (4) identify risks (e.g., mortality risk, significant cardiac event risk, hyperglycemia risk, hypoglycemia risk, etc.) associated with a current diabetes diagnosis, and (5) make patient-specific treatment recommendations for diabetes diagnosis and management. For example, decision support system 100 may perform method 400 by monitoring one or more analytes of a kidney disease patient during a plurality of time periods, the one or more analytes including at least glucose. The decision support system 100 may then determine one or more glucose metrics for the kidney disease patient, generate, using a trained machine learning model, a diabetes diagnosis based on the one or more glucose metrics and then generate one or more recommendations for treatment based on the diabetes diagnosis.

In certain embodiments, decision support system 100 presented herein may be configured to identify that a patient is experiencing diabetes, even in cases where the patient is not aware of the condition, or even when existing diagnostic methods, techniques, and tests do not indicate a diabetes diagnosis. In particular, in patients with kidney disease, diabetes and reduced glucose homeostasis may be masked by the kidney disease, whereby existing diagnostic methods, techniques, and tests may not diagnose the patient with diabetes and reduced glucose homeostasis. Thus, by identifying or predicting the presence and/or severity of diabetes for a patient with kidney dysfunction based on sensor data (e.g., generated by at least a continuous glucose sensor 202), decision support system 100 presented herein offers diagnosis that may be critical to the detection and management of diabetes. Method 400 is described below with reference to FIGS. 1 and 2 and their components.

In certain embodiments, decision support engine 114 of decision support system 100 may use various algorithms or artificial intelligence (AI) models, such as machine-learning models, trained based on patient-specific data and/or population data to provide diabetes diagnosis. The algorithms and/or machine-learning models may take into account one or more inputs 128 and/or metrics 130 described with respect to FIG. 3 for a patient when predicting whether the patient is experiencing, or is likely to experience, diabetes.

In certain embodiments, the one or more machine learning models may include multiple input, single output (MISO) models that are trained to predict the risk or likelihood of a patient experiencing diabetes, type, or severity. For example, one model may be trained to predict the likelihood of the present or future occurrence of diabetes, while another model may be trained to predict the likelihood of the present or future occurrence of Type II diabetes, and yet another model may be trained to predict the likelihood of the present or future occurrence of Type II insulin-dependent.

As an illustrative example, a MISO model may be trained to predict the type of diabetes for the patient. In certain embodiments, such a MISO model may be trained to output a vector having multiple values, where each value corresponds to a likelihood of a type of diabetes the patient may currently or in the future experience. For example, a vector output by a MISO model may include two values, each value corresponding to a different type of diabetes. For example, a first value that indicates 90% may show that there is a 90% chance that the patient is experiencing Type II diabetes or that the patient has a 90% risk or likelihood of experiencing Type II diabetes within a predefined time period. Meanwhile, a second value that indicates 0% may show that there is a 0% chance that the patient is experiencing Type I diabetes or that the patient has a 0% risk or likelihood of experiencing Type I within a predefined time limit.

In certain embodiments, the one or more machine learning models may include multiple input, multiple output (MIMO) models that are trained to predict the risk or likelihood of the presence, type, and severity of diabetes for a patient. For example, a single model may be trained to predict the likelihood of the present or future occurrence of diabetes, its type, and severity.

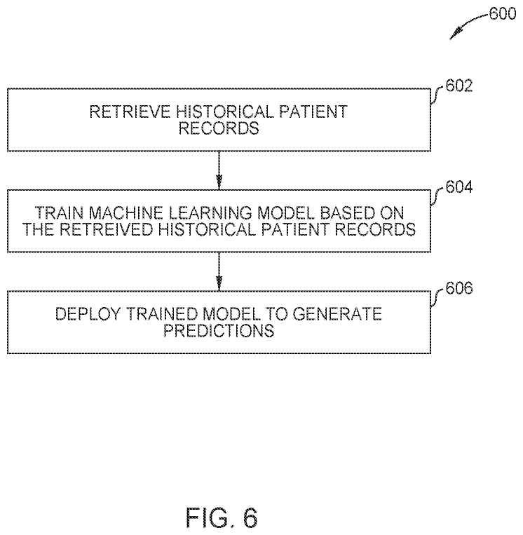

The one or more machine-learning models described herein for making such predictions may be initially trained using population data. A method for training the one or more machine learning models may be described in more detail below with respect to FIG. 6.

In certain embodiments, as an alternative to using machine learning models, decision support engine 114 may use rule-based models to predict the risk or likelihood of a patient having diabetes, its type, severity. Rule-based models involve using a set of rules for analyzing data. These rules are sometimes referred to as ‘If statements’ as they tend to follow the line of ‘If X happens then do or conclude Y’. In particular, decision support engine 114 may apply rule-statements (e.g., if, then statements) to predict the presence of diabetes.

Such rules may be defined and maintained by decision support engine 114 in a reference library. For example, the reference library may maintain ranges of metrics which may be mapped to different risks for diabetes. In certain embodiments, such rules may be determined based on empirical research or an analysis of historical patient records, such as the records stored in historical records database 112. In some cases, the reference library may become very granular. For example, other factors may be used in the reference library to create such “rules”. Other factors may include gender, age, diet, disease history, family disease history, body mass index (BMI), etc. Increased granularity may provide more accurate outputs.

At block 402, method 400 begins by continuously monitoring one or more analytes of a kidney disease patient, such as user 102 illustrated in FIG. 1, during a plurality of time periods to obtain analyte data. The one or more analytes monitored may include at least glucose. Block 402 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a continuous glucose sensor 202 configured to measure the patient's glucose levels during a plurality of time periods.

While the main analyte for measurement described herein is glucose, in certain embodiments, other analytes may be considered. In particular, combining glucose data with additional analyte data may help to further inform the analysis around diagnosis of diabetes. For example, monitoring additional types of analytes such as potassium, lactate, pyruvate, insulin and/or ketones measured by continuous analyte monitoring system 104, may provide additional insight into the generation of diabetes diagnosis.

The additional insight gained from using a combination of analytes, not just glucose, may increase the accuracy of the diabetes diagnosis. For example, the probability of accurately generating a diabetes diagnosis may be a function of the number of analytes measured for a patient. For example, the probability of accurately diagnosing diabetes using only glucose data may be less than a probability of accurately diagnosing diabetes using glucose and potassium data, which may also be less than a probability of accurately diagnosing diabetes using glucose, potassium, and lactate data for analysis.

For example, in certain embodiments, at block 402, continuous analyte monitoring system 104 may continuously monitor glucose and potassium levels of a patient during a plurality of time periods. In certain embodiments, the measured potassium concentrations may be used in conjunction with glucose levels for generating a diabetes diagnosis. In certain embodiments, potassium data may indicate the presence of kidney disease. Potassium data may also, in certain embodiments, indicate a change in the severity of kidney disease. A change in the presence and/or severity of kidney disease may indicate a change in (e.g., higher) risk of diabetes for a user based on the user's glucose metrics. For example, potassium data may indicate a user has worsening kidney disease and, as a result, the user's glucose metrics may be mapped to a higher risk of diabetes.

In addition to continuously monitoring one or more analytes of a patient during a plurality of time periods to obtain analyte data at block 402, optionally, in certain embodiments, method 400 may also include monitoring other sensor data (e.g., non-analyte data) during the plurality of time periods using one or more other non-analyte sensors or devices (e.g., such as non-analyte sensors 206 and/or medical device 208 of FIG. 2).

As mentioned previously, non-analyte sensors and devices may include one or more of, but are not limited to, an insulin pump, a haptic sensor, an ECG sensor and/or heart rate monitor, a blood pressure sensor, a respiratory sensor, a peritoneal dialysis machine, a hemodialysis machine, sensors or devices provided by display device 107 (e.g., accelerometer, camera, global positioning system (GPS), heart rate monitor, ECG sensor, etc.) or other user accessories (e.g., a smart watch), or any other sensors or devices that provide relevant information about the user. Metrics, such as metrics 130 illustrated in FIG. 3, may be calculated using measured data from one or more of these additional sensors. As illustrated in FIG. 3, metrics 130 calculated from non-analyte sensor or device data may include heart rate (including heart rate variability), respiratory rate, etc. In certain embodiments, described in more detail below, metrics 130 calculated from non-analyte sensor or device data may be used to further inform the analysis around generation of diabetes diagnosis.

In certain embodiments, one or more of these non-analyte sensors and/or devices may be worn by a user to aid in the detection of periods of increased physical exertion by the user. Such non-analyte sensors and/or devices may include an accelerometer, an ECG sensor, a blood pressure sensor, a heart rate monitor, an impedance sensor, a dialysis machine, and the like. Glucose levels may fluctuate during exercise and based on the type and intensity of exercise. Because of these extraneous fluctuations in glucose levels due to exercise, in certain embodiments, glucose metrics obtained for the user may exclude glucose levels during exercise. In particular, glucose data measured and collected from periods of increased physical exertion may be excluded from calculations of one or more glucose metrics used to predict diabetes. For example, glucose data collected during exercise may be excluded from calculations of TIR, allowing a diabetes diagnosis to be based on TIR when a user is not exercising.

In certain embodiments, one or more of these non-analyte sensors and/or devices may be worn by a user to aid in the detection of periods of treatment. Such non-analyte sensors and/or devices may include an accelerometer, an ECG sensor, a blood pressure sensor, a heart rate monitor, an impedance sensor, a dialysis machine, and the like. As part of a dialysis treatment, large amounts of glucose may enter a patient's system. This additional glucose may confound glucose metrics used to diagnose diabetes. Therefore, in certain embodiments, glucose data measured and collected from periods of treatment may be excluded from calculations of one or more glucose metrics used to predict diabetes. For example, glucose data collected during dialysis may be excluded from calculations of a glycemic variability measure and a diabetes diagnosis may be based on this glycemic variability measure without dialysis.

At block 404, method 400 continues by processing the analyte data to determine one or more glucose metrics for the kidney disease patient. Block 404, in certain embodiments, may be performed by decision support engine 114.

In certain embodiments, a glucose metric refers to a time-stamped glucose measurement or value. A glucose metric may refer to a change between one or more time-stamped glucose levels or values. For example, a glucose metric may be the difference between a glucose level at a first timestamp and a glucose level at a subsequent time stamp. A glucose metric may further be a glucose rate of change which refers to a rate that indicates the change of one or more time-stamped glucose levels in relation to one or more other time-stamped glucose levels. For example, a glucose metric may be the derivative of the change between a glucose level at a first timestamp and the glucose level at one or more subsequent timestamps.

Additionally or alternatively, time-stamped glucose levels may reflect the time of day the levels were recorded, and may be classified as a daytime glucose level (e.g., between 6 AM and 10 PM) or a nighttime glucose value (e.g., between 10 PM and 6 AM). The daytime glucose values may be compared with nighttime glucose values to determine how daytime glucose levels affect nighttime glucose levels.

In certain embodiments, a glucose metric indicates a mean glucose level, which may be an average of two or more time-stamped glucose levels. In certain embodiments, mean glucose may be calculated based on glucose levels as well as other inputs 128, such as food consumption information, whereby corresponding glucose levels and food consumption information (e.g., with overlapping timestamps) may be used to determine mean glucose. Mean glucose may be calculated over a period of time (e.g., one day) and compared to mean glucose of subsequent days.

Mean glucose is an average of one or more glucose levels over a given time period. Mean glucose may be used to calculate a glucose management indicator (GMI). Mean glucose and GMI have the known relationship:

$$GMI ( \% ) = 3.31 + 0.02392 \times [ mean glucose ( \frac{mg}{dL} ) ]$$

Glucose management indicator (GMI) is a measure derived from mean glucose values (i.e., average glucose level levels) and intended to convey the current status of glucose management. The relationship between mean glucose and GMI is derived from the relationship between continuous glucose sensor-measured glucose levels and clinical A1C. In certain embodiments, the glucose metric refers to a glucose management indicator (GMI). In certain embodiments, a GMI is calculated based on the relationship between GMI and mean glucose, especially where mean glucose has been determined based on glucose levels over a period of time. For example, a GMI may be calculated based on mean glucose levels over two weeks or one month. In certain embodiments, GMI may be calculated based on glucose levels as well as other inputs 128, such as food consumption information, whereby corresponding glucose levels and food consumption information (e.g., with overlapping timestamps) may be used to determine GMI.

In certain embodiments, a glucose metric may indicate glycemic variability, which may be the standard deviation of mean glucose (e.g., the standard deviation of the average of one or more time-stamped glucose levels in relation to the average of one or more other time-stamped glucose levels). For example, the standard deviation of mean glucose may be based on an average of two or more time-stamped glucose levels. In another example, the standard deviation of mean glucose may be based on an average of glucose levels over two or more periods of time. In yet another example, standard deviation of mean glucose may be based on an average of glucose levels over a certain time period. In certain embodiments, glycemic variability may be calculated based on glucose levels as well as other inputs 128, such as food consumption information, whereby glucose levels and food consumption levels with overlapping timestamps may be used to determine glycemic variability.

In some embodiments, a glucose metric indicating glycemic variability may be a set point metric. For example, decision support engine 114 may determine a set point based on an estimation of the “mode” of glucose values for a patient (e.g., the glucose value that appears the most often in a set of glucose values). The set point may be determined based on historical population data and/or the patient's historical glucose data, for example. Based on the calculated set point, the glucose metric may further indicate the time in range of glucose levels within a range of the set point value.

In certain embodiments, a glucose metric may be minimum and/or maximum glucose levels. For example, the minimum and/or maximum glucose level may be based on the minimum and maximum glucose levels over a day or a week, for example.

In certain embodiments, a glucose metric indicates a period of time glucose levels are within a range (e.g., glucose levels met, exceed, or are between threshold levels). Periods of time wherein glucose is within a specified range may be aggregated to determine the glucose time in range (TIR) for a specified range. For example, all of the periods of time wherein glucose levels are within a healthy glucose range may be aggregated over a monitoring period for which glucose data has been collected, such that the TIR for healthy range may be determined. In another example, all of the periods of time wherein glucose levels are below a healthy glucose range (e.g., low glucose range) over a monitoring period (e.g., two, four, eight weeks) may be aggregated to determine glucose TIR for a low range. In yet another example, all of the periods of time wherein glucose levels are above a healthy glucose range (e.g., high glucose range) over a monitoring period (e.g., two, four, eight weeks) may be aggregated to determine glucose TIR for a high range. In certain embodiments, glucose ranges, such as a healthy glucose range, a low glucose range, and/or a high glucose range, may be provided through user input or obtained from certain data stores (e.g., electronic medical records (EMRs), historical data, etc.).

In certain embodiments, a glucose metric may demonstrate patterns or trends in glucose levels obtained at different time points (e.g., 5 minutes apart, 10 minutes apart, etc.). The glucose metric may be an autocorrelation feature, demonstrating the similarities in patterns and trends between glucose levels obtained at different time points. The autocorrelation feature may be a numerical value between 1.0 and 0.0, where 1.0 demonstrates the time-series glucose levels are correlated (e.g., the patterns of glucose levels obtained at different time points are very similar and/or the same) and 0.0 demonstrates the time-series glucose levels are not correlated (e.g., the patterns of glucose levels obtained at different time points are not similar and/or the same).

In certain embodiments, machine-learning models, described herein, used to provide diabetes predictions may include one or more features not only related to glucose levels of the patient, but also to the above described glucose metrics of the patient. For example, an example machine-learning model may include weights applied to features associated with the one or more glucose metrics of the patient. Thus, in certain embodiments, prior to use of the machine-learning model, one or more glucose metrics for the patient may be calculated for input into the model.

Further, in certain embodiments, rule-based models, described herein, used to provide diabetes predictions, may include one or more rules not only related to glucose levels of the patient, but also glucose metrics of the patient. For example, a reference library, used to define one or more rules for the rule-based models, may maintain ranges of glucose levels and ranges of glucose metrics which may be mapped to different risk levels for diabetes. Thus, prior to use of the rule-based model, one or more glucose metrics for the patient may be calculated for input into the model. Additional features, such as additional glucose metrics, added to the model may, in some cases, allow for a more accurate prediction of diabetic disease for the patient.

At block 406, method 400 continues by generating a disease prediction using the analyte data associated with the one or more analytes and at least the one or more glucose metrics. Block 406 may be performed by decision support engine 114 illustrated in FIG. 1, in certain embodiments. As previously discussed, for patients with kidney disease, A1C is not a reliable measure for a diabetes diagnosis because the calculation A1C relies on an assumption about red blood cell half-life which is likely inaccurate. As such, glucose metrics determined at block 404 can be used by decision support system 100 to provide an accurate diabetes diagnosis for kidney disease patients. Based on a diabetes diagnosis, a recommendation to diagnose the patient with diabetes may be provided to an HCP.

As mentioned, different methods for generating a disease prediction may be used by decision support engine 114. In particular, in certain embodiments, decision support engine 114 may use a rule-based model to provide a real-time diabetes prediction, including providing diabetes diagnosis and staging. In particular, decision support engine 114 may apply rules to assess the presence and severity of diabetes in a patient, and/or identify risks associated with a current diabetes diagnosis of the patient (e.g., mortality risk, etc.).

For example, one rule may be related to a glucose level for the patient currently, or based on changes of the glucose level over time. Another rule may be based on a glucose metric, or changes of the glucose metric over time. Another rule may be based on TIR or on changes of TIR of a patient over time. Another rule may be based a mean glucose or on changes of the mean glucose of a patient over time. Another rule may be based on a GMI or on changes of the GMI of a patient over time. Another rule may be based on glycemic variability or on changes of glycemic variability of a patient over time. Another rule may be related to glucose level rates of change for the patient, such as “rapidly increasing” or “rapidly decreasing” rates of change (e.g., as described with respect to FIG. 3), or based on changes of the glucose level rates of change over time. Another rule may be related to a patient's glucose response, or lack thereof, to biochemical hypoglycemia (e.g., below 70 mg/dL), with or without levels of circulating insulin. Another rule may be related to a patient's glucose response, or lack thereof, to biochemical hyperglycemia (e.g., above 150 mg/dL), with or without levels of circulating insulin. Another rule may be related to metrics, insulin metrics, potassium metrics, lactate metrics, pyruvate metrics, and/or ketones metrics as described with respect to FIG. 3 or based on changes of such metrics over time. It is also contemplated that any of the rules may be used alone or in combination with any other rules by decision support system 100 to determine a diabetes diagnosis based on a patient's glucose metrics.

Such rules may be defined and maintained by decision support engine 114 in a reference library. For example, the reference library may maintain ranges of glucose levels and/or glucose metrics which may be mapped to different severities, types, and/or stages of diabetes. In certain embodiments, such rules may be determined based on empirical research as well as analyzing historical patient records from historical records database 112.

In certain embodiments, as an alternative to using a rule-based model, AI models, such as machine-learning models may be used to provide a real-time decision disease prediction. For example, decision support engine 114 may deploy one or more of these machine learning models for performing diagnosis and staging of diabetes in a kidney patient. In another example, one or more machine learning models may risk stratify a patient to identify a health risk associated with a current diagnosis of diabetes in a kidney disease patient. Risk stratification may refer to the process of assigning a health risk (e.g., risk of hyperglycemia and/or hypoglycemia) for a patient with diabetes. Identification of patients with a high risk of hyperglycemic and/or hypoglycemia may be used to generate treatment recommendations.

In particular, decision support engine 114 may obtain information from a user profile 118 associated with a patient, stored in user database 110, featurize information for the patient stored in user profile 118 into one or more features, and use these features as input into such models. Alternatively, information provided by the user profile 118 may be featurized by another entity (e.g., another server or computing device) and the features may then be provided to decision support engine 114 to be used as input into the ML models. In certain embodiments, features associated with the patient may be used as input into one or more of the models to assess the presence and severity of diabetes in the patient.

In certain embodiments, features associated with the patient may be used as input into one or more of the models to provide a disease prediction, which may involve identifying whether there is a high or low risk of the patient developing diabetes in the future. In certain embodiments, features associated with the patient may be used as input into one or more of the models to identify risks (e.g., mortality risk etc.) associated with a current diabetes diagnosis of the patient. In certain embodiments, features associated with the patient may be used as input into one or more of the models to perform any combination of the above-mentioned functions. Details associated with how one or more machine-learning models can be trained to provide real-time decision support for diabetes diagnosis and staging are further discussed in relation to FIG. 6.

As mentioned, in certain embodiments, other analyte data, in addition to glucose, may be used by decision support engine 114 to generate a disease prediction for a patient, at block 406. Analyte data, including potassium data, lactate data, pyruvate data, insulin data, cystatin C data, and/or ketone data (e.g., from measurements by continuous analyte monitoring system 104), may be used as input into such machine learning models and/or rule-based models to predict the presence, type and/or severity of diabetes of a user.

Decision support engine 114 may use the machine learning models and/or the rule-based models to generate a disease prediction based on continuous analysis of data (e.g., analyte data and, in some cases, non-analyte data) for the patient collected over various time periods. Analysis of data collected for the patient over various time periods may provide insight into whether the health and/or a disease of the patient is improving or deteriorating. For example, a patient previously diagnosed with diabetes, using the models discussed herein, may continue to be constantly monitored (e.g., continuously collect for the patient) to determine whether the disease is getting worse or better, etc. As an example, a comparison of glucose levels, time-stamped glucose levels, glucose baselines, absolute maximum glucose levels, absolute minimum glucose levels, glucose level rates of change, glucose metrics (e.g., glucose set point metrics, glucose autocorrelation features, etc.), TIR, mean glucose, GMI, and/or glycemic variability for a patient over multiple periods of time (e.g., days, weeks, months, etc.) may be indicative of the patient's disease progression. For example, decision support engine 114 may determine a patient's kidney disease is progressing if a patient's absolute minimum glucose levels begin to decrease over time, especially when the patient is asleep.

In certain embodiments, decision support engine 114 may provide a disease prediction based on the patient's time-stamped glucose values. For example, a kidney disease patient may have higher daytime glucose levels, more daytime hyperglycemic events, lower nighttime glucose levels, and/or more nocturnal hypoglycemic events over time, which may demonstrate worsening glycemic control, and therefore, worsening kidney disease. Additionally, a patient with kidney disease may experience a hyperglycemic spike after dinner and/or when the patient goes to sleep. If the patient's hyperglycemic spike after dinner and/or when the user goes to sleep increases in magnitude over time, decision support engine 114 may determine the patient is experiencing a worsening kidney disease.

In certain embodiments, a lower autocorrelation feature (e.g., less than 0.5) may demonstrate worsening kidney disease. In certain embodiments, higher maximum glucose levels and lower minimum glucose levels may demonstrate worsening kidney disease. In certain embodiments, decision support engine 114 may use a set point metric to determine a patient's kidney disease stage. For example, as kidney disease worsens, there is increased variability in glucose measurements, which may result in less glucose level time in range, specifically within a range of a set point. As glucose levels within a range of the set point become less frequent, decision support engine 114 may determine a user's kidney disease is progressing.

In certain embodiments, the rates of change and the directional movement of glucose and potassium in concordance and in opposite concordance with each other over time and/or the time delay of one moving to affect the other can be (1) used in identifying an improvement in the user's diabetes and/or kidney disease and can also (2) act as a surrogate about when insulin becomes active to push potassium and glucose into the cells but also helpful to rule out certain other conditions. Similarly, differences in the rate of change between potassium and glucose can be used to derive insight because, under normal conditions (e.g., when the patient is not exercising and/or in a disease or injury state), potassium seems to change at a slower rate than glucose does. That time delay may be a predictor of potassium changes or the length of time from the glucose to potassium change may be an indicator of insulin resistance or another factor. For example, a shorter time delay versus a longer time delay may indicate reaction kinetics of uptake have changed from one intracellular pathway to another. Therefore, a change in the time delay may indicate a change in the user's diabetes and/or kidney disease condition.

In some cases, method 400 continues at block 408 by decision support engine 114 generating one or more recommendations for management and/or treatment, based, at least in part, on the disease prediction generated at block 406. In particular, decision support engine 114 may provide recommendations for management and/or treatment of diabetes, such as lifestyle recommendations, medication recommendations, medical intervention recommendations, or other recommendations for managing diabetes and glucose homeostasis. Decision support engine 114 may output such recommendations for treatment to the user (e.g., through application 106). In certain other cases, method 400 ends at block 406 with a disease prediction for a patient (e.g., block 408 may be optional).

In certain embodiments, decision support engine 114 may use one or more other machine-learning models, trained based on patient-specific data and/or population data, to provide management and/or treatment recommendations for a user. The machine-learning models may take into account one or more inputs 128 and/or metrics 130 (e.g., including glucose metrics) described with respect to FIG. 3 for a patient to determine optimal recommendations for management and/or treatment of the patient's diabetes. In certain embodiments, the model may look at different patterns of analyte measurements collected for the patient to guide the patient in the management and/or treatment of their disease.

In certain embodiments, as an alternative to using machine-learning models, decision support engine 114 may use one or more decision trees to provide management and/or treatment recommendations. The decision tree may be rule-based and provide a recommendation regarding the management and/or treatment of the patient's diabetes based on one or more rules.

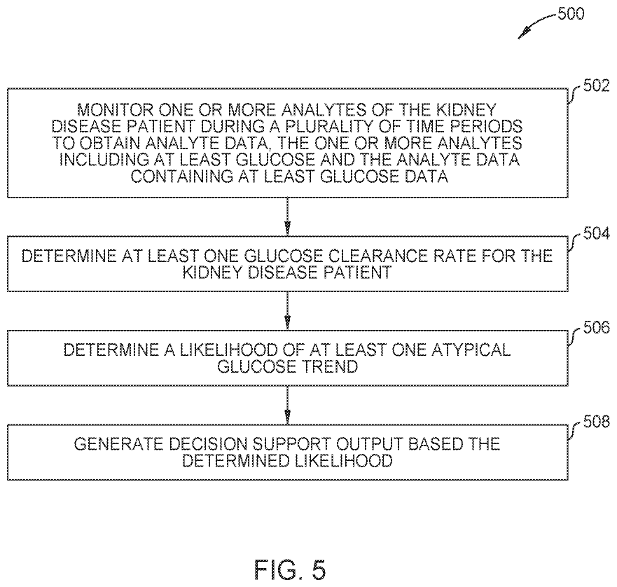

Recommendations for the management and/or treatment of diabetes may, in some cases, be based on a determined glucose metric (e.g., glucose levels, rates of change of glucose levels, TIR, mean glucose, GMI, glycemic variability and/or glucose clearance rate), and/or a change in the glucose metric of the patient. For example, a recommendation for the management and/or treatment of diabetes may be based on a low TIR, a high GMI, and/or a high glycemic variability. Recommendations regarding management and treatment of diabetes may include diet, exercise, lifestyle, treatment, insulin administration, and/or medication recommendations, as discussed in further detail with respect to FIG. 5.

In certain embodiments, a diabetes management and/or treatment intervention recommendation may include a recommendation for the patient to seek medical aid. For example, in certain embodiments, the diabetes management and/or treatment recommendation may indicate to a patient that the patient needs to immediately go to the emergency room and/or contact their physician. In certain other embodiments, a management and/or treatment recommendation may automatically alert the HCP of the patient as to the condition of the patient for intervention by the HCP. In certain other embodiments, a diabetes management and/or treatment recommendation may alert medical personnel to send aid to the patient, e.g., trigger ambulance services or paramedic services to provide urgent pre-hospital treatment and stabilization to the patient and/or transport of the patient to definitive care. In certain embodiments, decision support engine 114 may make a management and/or treatment recommendation based on a patient's ability to seek medical help and/or the accessibility of the patient to medical help.

Note that although method 400 is described in relation to providing diabetes decision support to a kidney disease patient, in certain embodiments, method 400 may be performed similarly for patients without kidney disease. The average glucose would be expected to be similar between patients without and without kidney disease (e.g., CKD). However, glycemic variability would likely be higher in patient with CKD than without, especially in those with stage 3 or 4 CKD (advanced CKD) and/or on dialysis. In addition A1C may be artificially low in such patients and, therefore, a “mean shift” will be observed and likely unstable depending on the therapies administered to such patients. While in people with diabetes and without CKD the relationship between average glucose measured by a continuous glucose monitor and A1C is consistent, many of the therapies (e.g., erythropoietin (EPO) administration, anemia, dialysis) for CKD can impact red blood cell metabolism. Therefore, for CKD patients a higher glycemic variability, a similar average glucose value, and very little consistent relationship over time to A1C can be expected.

FIG. 5 is a flow diagram illustrating an example method 500 for providing decision support using a continuous analyte sensor including, at least, a continuous glucose sensor 202, in accordance with certain example aspects of the present disclosure. For example, method 500 may be performed to provide decision support to a user, using a continuous analyte monitoring system 104 including, at least, a continuous glucose sensor 202, as illustrated in FIGS. 1 and 2.

Method 500 may be performed by decision support system 100 to collect/generate data such as inputs 128 and metrics 130, including for example, analyte data, patient information, and non-analyte sensor data mentioned above, to (1) automatically detect and determine glucose metrics, including, for example, a glucose clearance rate, (2) assess the presence and severity atypical glucose trends, (3) risk stratify patients to identify those patients with a high risk of hyperglycemic and/or hypoglycemia, (4) identify risks (e.g., mortality risk, significant cardiac event risk, etc.) associated with current glucose metrics, (5) make patient-specific treatment decisions or recommendations for glucose, diabetes, and kidney disease management, and (6) provide information on the effect of an intervention (e.g., an effect of a lifestyle change of the patient, an effect of a surgical procedure, an effect of the patient taking new medication, etc.). In other words, the decision support system presented herein offers information to direct and help improve care for patients with, or at risk, of diabetes and kidney disease.

Patient care may be improved by providing tailored recommendations to manage and treat diabetes in patients with kidney disease. As discussed, kidney disease affects glucose homeostasis by affecting the metabolism of glucose. A user's glucose clearance rate and/or other metrics may be used to approximate the user's metabolism of glucose at their current kidney function and improve management of glucose homeostasis. Specifically, as described below in relation to method 500, a user's glucose clearance rate may be used in conjunction with other glucose metrics, including current glucose levels and rates of change, to give context to glucose trends for that user. Glucose clearance rate may reveal when glucose levels are trending towards an adverse event (e.g., hyperglycemia and/or hypoglycemia) for the user and their current kidney function. For example, a rapidly increasing glucose trend and a slow glucose clearance rate may indicate the user is at risk of hyperglycemia. Alternatively, a user with kidney disease may experience a rapidly decreasing glucose trend as a result of low insulin clearance, reduced glucose consumption by the kidneys, and reduced kidney gluconeogenesis, which may indicate the user is at risk of hypoglycemia. Treatment recommendations may then be given to the user to manage the adverse event, as described in relation to method 500. Method 500 is described below with reference to FIGS. 1 and 2 and their components.

At block 502, method 500 begins by continuously monitoring one or more analytes of a patient, such as user 102 illustrated in FIG. 1, during a plurality of time periods to obtain analyte data. The one or more analytes monitored may include at least glucose. For example, block 502 may be performed by continuous analyte monitoring system 104 illustrated in FIGS. 1 and 2, and more specifically, continuous analyte sensor(s) 202 illustrated in FIG. 2, in certain embodiments. Continuous analyte monitoring system 104 may comprise a continuous glucose sensor 202 configured to measure the patient's glucose levels.

Similar to method 400 of FIG. 4, while the main analyte for measurement described herein is glucose, in certain embodiments, other analytes may be considered. In particular, combining glucose levels with additional analyte data may help to further inform the analysis around managing glucose homeostasis. For example, monitoring additional types of analytes, such as potassium, lactate, pyruvate, insulin and/or ketones measured by continuous analyte monitoring system 104, may be used to provide additional insight into glucose homeostasis, and/or supplement information used to determine optimal treatment for maintaining glucose homeostasis. The additional insight gained from using a combination of analytes, and not just glucose, may increase the accuracy of glucose management, as described in relation to method 400.

As mentioned above, kidney dysfunction affects at least glucose, insulin and potassium metabolism. Therefore, the additional insights gained from potassium levels may improve analysis of glucose clearance rate at block 504 and atypical glucose trends at block 506, because glucose homeostasis and risk of adverse events are affected by level of and changes to kidney functioning. For example, potassium levels could indicate worsening kidney dysfunction, which negatively impacts glucose homeostasis and increases risk of adverse hypoglycemic/hyperglycemic events. Alternatively, glucose metrics and corresponding trends derived from glucose metrics may indicate worsening kidney function, which negatively impacts potassium homeostasis and increases risk of hypokalemic/hyperkalemic events. Additionally, a change in kidney function may impact treatment recommendations because treatment efficacy may depend on kidney function.

In another example, lactate levels may be associated with glucose, insulin, and potassium metabolism. Lactate levels may be used to detect consumption of food, exercise, rest, and/or stress. Therefore, the additional insights gained from lactate levels may improve analysis of the glucose clearance rate at block 504 and atypical glucose trends at block 506 by associating glucose metrics with different body states (e.g., food consumed, exercise, rest, stress, etc.).

Additionally, pyruvate is used in gluconeogenesis to produce glucose. In kidney dysfunction, kidney gluconeogenesis is impaired, which reduces glucose homeostasis. Pyruvate levels may, therefore, provide insight into gluconeogenesis and the resulting increase in glucose level. For example, increasing pyruvate levels may indicate an expected rise in glucose levels, which may improve determination of glucose metrics. Accordingly, given the interaction of the comorbidities discussed above, parameters, thresholds, and/or rules associated with decision support algorithms and/or models may be altered based, on a number of analytes being measured for input to reflect the knowledge attained from each of the other analytes being measured and/or morbidities associated with the additional analytes being measured.

In addition to continuously monitoring one or more analytes of a patient during a plurality of time periods to obtain analyte data at block 502, optionally, in certain embodiments, method 500 may also include monitoring other sensor data (e.g., non-analyte data) during the plurality of time periods using one or more other non-analyte sensors or devices (e.g., such as non-analyte sensors 206 and/or medical device 208 of FIG. 2).

As mentioned previously, non-analyte sensors and devices may include one or more of, but are not limited to, an insulin pump, a haptic sensor, an ECG sensor and/or heart rate monitor, a blood pressure sensor, a sweat sensor, a respiratory sensor, a thermometer, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user.

At block 504, method 500 continues by processing the analyte data to determine at least one glucose clearance rate of the patient. Block 504, in certain embodiments, may be performed by decision support engine 114. Block 504 in method 500 of FIG. 5 may be similar to block 404 in workflow 400 of FIG. 4.

In certain embodiments, a glucose clearance rate may be determined from sensor data (e.g., glucose levels obtained from a continuous glucose sensor 202 of continuous analyte monitoring system 104). For example, the glucose clearance rate may be determined by a glucose challenge that may test a user's metabolic function in order to determine a user's response to the consumption of a known amount of glucose (e.g., glucose clearance rate). A glucose challenge introduces a known or estimated amount of glucose to the body for the purpose of measuring the resulting increasing and/or decreasing rate of change in glucose level or other analyte levels. A glucose clearance rate may indicate the kidney's ability to metabolize glucose including the capacity to react to increasing and/or decreasing glucose levels. Therefore, the glucose clearance rate for a user with kidney disease may include a more rapid increase in glucose levels and a higher peak glucose level in response to the introduction of glucose when compared to a healthy user. A user with kidney disease may have a faster and more prominent glucose peak following the introduction of glucose due to the kidney's reduced glucose filtration capacity and reduced glucose consumption.

The user's response to the challenge may be obtained and recorded in the form of absolute glucose levels, changes in glucose levels, rates of change of glucose levels, mean glucose, GMI, TIR, glycemic variability and/or any other glucose metric. Glucose levels may be determined at specified intervals (e.g., 5 minutes, 10 minutes, 30 minutes, 1 hour, 2 hours, etc., following consumption of glucose), over a period of time (e.g., 2 hours following consumption of glucose), at specified times (e.g., morning, evening, nighttime), etc. In certain embodiments, a glucose clearance rate may be determined by calculating a slope between an initial high glucose value (e.g., resulting increase associated with consumption of a known or estimated amount of glucose) and a subsequent low glucose value. In certain embodiments, glucose clearance rates following administration of certain types of insulin, such as rapid acting insulin, may be obtained and recorded. The expectation would be that in advanced stages of CKD, the impaired insulin clearance would show up as a longer glucose clearance time and possibly higher clearance rate.

In certain embodiments, consumption of a known amount of glucose may be based on a recommendation provided by decision support engine 114. Decision support engine 114 may provide recommendations for a glucose challenge by recommending consumption of the known amount of glucose to increase glucose levels. For example, a glucose challenge recommendation may be to consume 25 grams of glucose. In certain embodiments, glucose challenge recommendations may be to consume specific food items. For example, a recommendation may be to consume an apple. In certain embodiments, glucose challenge recommendations may include a recommendation for the patient to consume a meal with a certain meal composition. For example, a glucose challenge recommendation may be to consume a high carb meal, such as 60% total carbohydrate.

Further, in certain embodiments, a glucose challenge recommendation may include a recommendation for the user to input food consumption information manually, through an application, by scanning a bar code or menu, or other input methods. For example, food consumption information about food (e.g., carbohydrate) that the user has already consumed may provide the known amount of glucose introduced for a glucose challenge. In certain embodiments, food consumption information may be provided as part of inputs 128 and may include information about one or more of meals, snacks, and/or beverages, such as one or more of the size, content (e.g., milligrams (mg) of glucose, carbohydrate, fat, protein, etc.), sequence of consumption, and time of consumption, as discussed above in relation to inputs 128. In certain embodiments, decision support engine 114 may prompt a user to confirm the consumption of glucose. For example, decision support engine 114 may ask the user to confirm a meal (e.g., “Did you consume a meal?”).

In certain embodiments, glucose consumption information may not be available or provided. In such embodiments, sensor data may be used to determine the amount of glucose consumed. Sensor data may indicate the user's glucose level, changes in the user's glucose level, rates of change of glucose level, and/or any other glucose metric. In certain embodiments, sensor data may include other analyte data, which combined with glucose data, may help to inform the amount of glucose consumed, the composition of a meal/food item consumed, food consumption information and/or otherwise indicate current or impending glucose metrics. Additional analytes may include potassium, lactate, ketones, pyruvate, dextrose, insulin, and/or any other analyte. For example, sensor data may include glucose levels and lactate levels which may indicate, or otherwise represent, food consumption information, allowing the determination of a glucose clearance rate based on the indicated glucose consumed and resulting rates of change of glucose.

In certain embodiments, where glucose consumption information is unknown, historical data may be used to determine the amount of glucose consumed. A user's own historical data may include previous sensor data, food consumption information, exercise information, time information, location information etc. In certain embodiments, historical data may indicate timing of consumption of glucose, including a known and/or unknown amount of glucose, composition of a consumed meal, and/or previous glucose clearance rates to be used in determining whether a user has consumed glucose and/or the amount or composition of glucose consumed when initiating a glucose challenge. For example, a user may consume a meal at the same time every day. In another example, a user may consume a similar composition of meal every day. In yet another example, a user may consume a similar composition of meal at a certain location. As another example, a user may consume a similar composition following an activity (e.g., user consumes a sports drink following exercise). As such, historical information can be used to predict or estimate the amount of glucose consumed to be used to determine a glucose clearance rate. For example, if a user's historical data indicates that typically a meal corresponding to a 20 mg/dL increase in glucose levels is consumed by the user every day for lunch, then when calculating the user's glucose clearance rate on a particular day, decision support engine 114 may predict that a similar amount of glucose will be consumed for lunch on that particular day.

In certain embodiments, glucose challenge notifications may be provided by decision support engine 114 to confirm the consumption of glucose based on historical data. For example, a glucose challenge notification may ask the user to confirm a meal (e.g., “Did you consume a meal?”). Further, in certain embodiments, a glucose challenge notification may include a recommendation for the user to input food consumption information manually, through an application, by scanning a bar code or menu, or other input methods.

In certain embodiments, the amount of glucose consumed, e.g., determined through any of the above discussed methods, is used to calculate the initial increase and subsequent decrease in glucose levels when determining a glucose clearance rate. For example, an amount of glucose is consumed and a glucose clearance rate is determined based on the resulting increase associated with the amount of glucose consumed and then the rate of reduction in glucose levels.

In certain embodiments, a glucose clearance rate may be determined based on not only glucose data obtained from a continuous glucose sensor 202 but also insulin administration information. For example, some users with known diabetes may be on exogenous insulin to reduce the risk of hyperglycemia. For users on exogenous insulin, a glucose challenge may include the administration of insulin to prevent hyperglycemia during the challenge. A user's glucose clearance rate determined by a glucose challenge may, therefore, be based on both the amount of glucose consumed and the dosage of insulin administered.

For users with kidney dysfunction and on exogenous insulin, administration of insulin may have an unexpected, atypical, or otherwise different impact on glucose clearance rate, especially when compared to users without kidney dysfunction. Also, as kidney dysfunction progresses or regresses, insulin administration may have a different impact on glucose clearance rate compared to previous glucose clearance rates associated with previous states of the user's kidney dysfunction. Therefore, for users with kidney dysfunction, a glucose clearance rate determined based on a glucose challenge involving insulin administration may provide additional insight into glucose homeostasis and risk of adverse events. A glucose clearance rate, for such glucose challenges, is calculated based on both the amount of glucose consumed and the dosage and type of insulin administered.

In certain embodiments, the consumption of glucose and administration of insulin includes consuming a known amount of glucose and administering a known dosage of insulin. A known amount of glucose may be determined based on any of the above-discussed methods. Information about administration of a known dosage of insulin (“insulin information”) may include information about the type, dosage amount, activity rate, activity duration, and/or timing of the insulin administrated as part of the challenge and may be provided as medication information, provided as part of inputs 128 of FIG. 1.

In certain embodiments, the type of insulin administered for a glucose challenge is rapid-acting insulin. In a patient without kidney dysfunction, the onset (e.g., the length of time for insulin to begin acting on blood glucose) is between 5-15 minutes with an activity peak between minutes, and a total duration of activity at 3 hours. For example, a user may consume a known amount of glucose and administer rapid-acting insulin as part of a glucose challenge, whereby the user's glucose clearance rate for rapid-acting insulin may be determined and correlated with insulin activity onset, peak, and duration. For users with kidney dysfunction, due to insulin resistance and impaired insulin clearance, the onset and activity peak of rapid-acting insulin may be more rapid, and the duration of activity may be extended. Thus, in users with kidney dysfunction, determining a glucose clearance rate associated with exogenous insulin may improve decision support by accounting for the altered effects of insulin on glucose levels when determining glucose trends and treatment recommendations.

In certain embodiments, an administration of insulin may be based on a recommendation provided by decision support engine 114. Decision support engine 114 may provide recommendations for an insulin dependent glucose challenge by recommending insulin administration. Recommendations may indicate the type, dosage, and/or timing of the insulin to be administered as part of the challenge. For example, a recommendation may be to administer units of rapid-acting insulin following a meal.

In certain embodiments, insulin administration may be determined using historical medication information, real-time medication information, or a combination thereof. For example, medication information may indicate a user typically administers insulin at certain times, in conjunction with certain activities and/or at certain glucose levels. Medication information, provided as part of inputs 128 of FIG. 1, may indicate a user has administered insulin as part of a glucose challenge to determine glucose clearance rate. In certain embodiments, notifications may be provided by decision support engine 114 to confirm the administration of insulin based on medication information. For example, a notification may ask the user to confirm administration (e.g., “Did you dose rapid-acting insulin?”). The confirmed insulin administration may then form part of a glucose challenge to determine glucose clearance rate.

In certain embodiments, medication information may be provided by a user through manual entry, by providing a photograph through an application that is configured to recognize insulin type and quantities, and/or by scanning a bar code. In some examples, the insulin information may be received via a convenient user interface provided by application 106. In certain embodiments, the insulin information may be received via a wireless connection on a smart pen and/or from an insulin pump

In certain embodiments, the administration of insulin may be determined using sensor data. Sensor data may include the user's glucose level, changes in the user's glucose level, rates of change of glucose level and/or any other glucose metric. In certain embodiments, sensor data may include other analyte data associated with one or more analytes other than glucose. Additional analytes may include potassium, lactate, ketones, pyruvate, dextrose, insulin, which may fluctuate as a result of insulin administration. Changes in one or more additional analyte levels combined with changes in glucose levels may indicate insulin administration. For example, decreasing glucose levels and potassium levels may indicate insulin has been administered. As a result, glucose data and additional analyte data may indicate that insulin has been administered and the type and/or dosage of the insulin, to be used as medication information for determining a glucose clearance rate.

In certain embodiments, a glucose challenge may be repeated at one or more times to determine a user's glucose clearance rate at different times, periodically, etc. As an example, a user's glucose clearance rate may be different for different activities (e.g., activity, exercise, sleep, eating, medication, etc.) and, therefore, in certain embodiments, multiple glucose clearance rates may be determined for a certain user subsequent to different user activities to obtain an average glucose clearance rate. Different activities may be automatically determined based on sensor data, including additional analyte data and non-analyte data. For example, lactate data may be used, in certain embodiments, to determine a user is exercising, consuming a meal, at rest and/or stressed. In certain embodiments, upon determining a glucose clearance rate for a variety of activities, an activity profile may be created for each of those activities, each activity profile storing the corresponding glucose clearance rate. For example, a user's glucose clearance rate for exercise may be determined and used to generate an exercise activity profile. Decision support provided to the user in relation to the user's exercise may then be determined based on the glucose clearance rate determined for the user's exercise profile.

In certain embodiments, a user's glucose clearance rate may be determined periodically. In another example, a user's glucose clearance rate may be determined at regular intervals (e.g., weekly, monthly, bimonthly, annually). In certain embodiments, a user's glucose clearance rate may be determined upon a change to user inputs 128 or metrics 130. For example, a user's glucose clearance rate may be determined following a change in disease status (e.g., worsening or improving kidney dysfunction). As discussed above, in certain embodiments, additional analytes (e.g., potassium) may indicate a change in disease status, based on which a glucose clearance rate associated with the change in disease status may be determined. In another example of a change to user inputs 128 or metrics 130, in certain embodiments, a user's glucose clearance rate may be determined following a change in medication or treatment information (e.g., initiation or cessation of a medication, change in medication type, dosage, timing etc.). Additionally, in certain embodiments, a user's glucose clearance rate may be determined after a delay following a change (e.g., 2 weeks after initiation of a new exercise program). In certain embodiments, a user's glucose clearance rate may be determined at user's request. For example, a user may request a glucose clearance rate determination via a convenient user interface provided by application 106.

In certain embodiments, an alert may be provided by decision support engine 114 in response to a change in a user's glucose clearance rate. For example, a decision support notification may be that a user's glucose clearance rate has increased or decreased since the previous glucose clearance rate determination.

In certain embodiments, any of the above described methods for determining glucose clearance rate, including any combination of the above described methods, may be used to determine a user's glucose clearance rate.

At block 506, method 500 continues by determining a likelihood of at least one atypical glucose trend associated with the glucose clearance rate for the patient with kidney dysfunction. As discussed, users with kidney disease may experience different (i.e., atypical) glucose trends compared to users without kidney disease. Additionally, glucose trends may have different (i.e., atypical) implications for users with kidney disease as compared to users without kidney disease. For example, for a user with kidney disease, an increasing glucose trend may indicate a more significant risk of hyperglycemia based on the glucose clearance rate associated with the user at their current kidney dysfunction. In another example, a decreasing glucose trend may indicate a more significant risk of hypoglycemia based on the glucose clearance rate associated with the user at their current kidney dysfunction. Block 506 may be performed by decision support engine 114 illustrated in FIG. 1, in certain embodiments.

As mentioned, patients with kidney disease may have reduced glucose homeostasis, diabetes, and/or higher risk of hyperglycemia and/or hypoglycemia as opposed to patients without kidney disease. Additionally, patients with kidney disease may have altered treatment effectiveness and/or treatment indications. Because of these effects of kidney disease, successful management of diabetes in patients with kidney disease may depend upon recognition of the presence and risk of atypical glucose trends and effective treatment options.

Different methods for determining a likelihood of an atypical glucose trend associated with glucose clearance rate may be used by decision support engine 114. In certain embodiments, decision support engine 114 may use a rule-based model to provide real-time decision support based on a determined likelihood of an atypical glucose trend. For example, decision support engine 114 may apply rule-statements to assess the presence of an atypical glucose trend in a patient, perform risk stratification for a patient (e.g., a patient has a high or low risk of hyperglycemia and/or hypoglycemia based on glucose clearance rate, and/or identify risks associated with a current atypical glucose trend for the patient. Any rule may be used alone and/or in combination with any other rule by decision support engine 114.

For example, one rule may be related to a patient's glucose clearance rate or changes in a patient's glucose clearance rate. Another rule may be based on a patient's glucose clearance rate and a current glucose level. For example, a prolonged glucose clearance rate and low glucose level may be an atypical glucose trend resulting in hypoglycemia. Another rule may be based on a patient's glucose clearance rate and rates of change of glucose level. For example, a prolonged glucose clearance rate and decreasing rapidly glucose level may be an atypical glucose trend resulting in hypoglycemia. Another rule may be based on a patient's glucose clearance rate and glucose metrics, TIR, mean glucose, GMI, and/or glycemic variability. A variety of other rules may be used that are based on a combination of the user's glucose clearance rate and other glucose metrics, such as a TIR, mean glucose, GMI, glucose variability, absolute maximum glucose level, absolute minimum glucose level, rates of change of glucose levels, as well as changes in such glucose metrics.

Certain rules may also be based on insulin administration information, such as historical insulin administration, whether the glucose clearance rate is exogenous-insulin dependent, and/or insulin on board. For example, an administration of insulin and a corresponding exogenous-insulin glucose clearance rate may indicate an atypical glucose trend resulting in hypoglycemia. In another example, a change in administration of insulin (e.g., skipped insulin dose) and a corresponding exogenous-insulin glucose clearance rate may indicate an atypical glucose trend resulting hyperglycemia.

Certain rules may also utilize a user's glucose clearance rate indicated by a user's activity profile based on a glucose clearance rate for a corresponding activity. For example, when a user is exercising, the user's glucose clearance rate, as indicated by the user's exercise activity profile, is used to determine a likelihood of an atypical glucose trend. Certain rules may also involve potassium metrics, lactate metrics, pyruvate metrics, and/or any sensor data as described with respect to FIG. 3.

Rules may be defined and maintained by decision support engine 114 in a reference library. For example, the reference library may maintain ranges of glucose levels, ranges of glucose level rates of changes, ranges of glucose clearance rates, etc., which may be mapped to different atypical glucose trends. In certain embodiments, such rules may be determined based on empirical research as well as analyzing historical patient records from historical records database 112.

In certain embodiments, as an alternative to using a rule-based model, AI models, such as machine-learning models may be used to provide real-time decision support for kidney disease diagnosis and staging. In certain embodiments, decision support engine 114 may deploy one or more of these machine learning models for performing determining likelihood of atypical glucose trends. In particular, decision support engine 114 may obtain information from a user profile 118 associated with a patient, stored in user database 110, to featurize information for the patient stored in user profile 118 into one or more features, and use these features as input into such models. Alternatively, information provided by the user profile 118 may be featurized by another entity and the features may then be provided to decision support engine 114 to be used as input into the ML models. In certain embodiments, features associated with the patient may be used as input into one or more of the models to assess the likelihood of atypical glucose trends in the patient.

In certain embodiments, features associated with the patient may be used as input into one or more of the models to identify whether there is a high or low risk of the patient developing an atypical glucose trend within a certain period of time. In certain embodiments, features associated with the patient may be used as input into one or more of the models to identify risks (e.g., hyperglycemia and/or hypoglycemia) associated with a current atypical glucose trend of the patient. Details associated with how one or more machine-learning models can be trained to provide real-time decision support for atypical glucose trends are further discussed in relation to FIG. 6.

As mentioned, in certain embodiments, other analyte data, in addition to glucose, may be used by decision support engine 114 to generate an atypical glucose trend for a patient, at block 506. Analyte data, including glucose and potassium data, lactate data, pyruvate data, insulin data, cystatin C data, or ketone data (e.g., from measurements by continuous analyte monitoring system 104), may be used as input into such machine learning models and/or rule-based models to predict an atypical glucose trend of a user.

Decision support engine 114 may use the machine learning models and/or the rule-based models to generate an atypical glucose trend based on continuous analysis of data (e.g., analyte data and, in some cases, non-analyte data) for the patient collected over various time periods. Analysis of data collected for the patient over various time periods may provide insight into the direction of the atypical glucose trend, such as whether the health of the patient is improving or deteriorating. For example, a patient with an atypical glucose trend may continue to be constantly monitored (e.g., continuously collect data for the patient) to determine whether the atypical glucose trend is improving or deteriorating (e.g., glucose levels are returning to a healthy range). As an example, comparison of atypical glucose trends, including treatment for atypical glucose trends, may be monitored over a period of time (e.g., minutes, hours, days, weeks, months), may indicate reduction in atypical glucose trends and/or increase in effectiveness of treatment (e.g., improvement in glucose homeostasis and/or diabetes and kidney disease management).

At block 508, method 500 continues by generating decision support output based on the determined likelihood of at least one atypical glucose trend. Block 508 may be performed by decision support engine 114 illustrated in FIG. 1, in certain embodiments.

In certain embodiments, the decision support output may include alerts of an adverse glycemic event, such as glucose levels exceeding thresholds, rates of change exceeding threshold, hyperglycemia and/or hypoglycemia. Decision support engine 114 may output such alerts to the user (e.g., through application 106). In certain embodiments, decision support engine 114 may use one or more machine-learning models, trained based on patient-specific data and/or population data, to provide alerts for an adverse glycemic event. The algorithms and/or machine-learning models may take into account the likelihood of at least one atypical glucose trend determined at block 508 as well as one or more inputs 128 and/or metrics 130 (e.g., including glucose metrics) described with respect to FIG. 3 for a patient to determine alerts for an adverse glycemic event.

In certain embodiments, the decision support output may include one or more recommendations for treatment, based on the determined likelihood of at least one atypical glucose trend. In particular, decision support engine 114 may provide recommendations for the treatment or prevention of an adverse glycemic event, such as medication recommendations, diet recommendations, lifestyle recommendations, medical intervention recommendations, or other recommendations for managing atypical glucose trends. Decision support engine 114 may output such recommendations for treatment to the user (e.g., through application 106).

In certain embodiments, decision support engine 114 may use one or more machine-learning models, trained based on patient-specific data and/or population data, to provide recommendations for the treatment and/or prevention of an atypical glucose trend. The algorithms and/or machine-learning models may take into account the likelihood of at least one atypical glucose trend determined at block 508 as well as one or more inputs 128 and/or metrics 130 (e.g., glucose metrics) described with respect to FIG. 3 for a patient to determine optimal recommendations for prevention and/or management of the patient's atypical glucose trend.

In certain embodiments, as an alternative to using machine-learning models, decision support engine 114 may use one or more decision trees to provide recommendations for the treatment or prevention of an adverse glycemic event. The decision tree may be rule-based and include one or more rules used to provide a recommendation regarding the management of the patient's atypical glucose trend.

Recommendations for the treatment or prevention of an atypical glucose trend may, in some cases, be based on optimal glucose levels and insulin levels determined for a patient. In particular, in certain embodiments, one or more algorithms may be used to determine an optimal balance of glucose and insulin levels of a patient that is then used to form one or more recommendations for the patient regarding treatment (e.g., insulin and/or medication), diet, and/or lifestyle. Furthermore, recommendations for treatment and/or prevention of atypical glucose trends may, in some cases, be based on management of kidney disease. Specifically, in certain embodiments, an optimal balance of potassium, glucose, and insulin levels for a patient may be determined and used to provide recommendations to the user for prevention of atypical glucose trends. As an example, a medication recommendation for insulin administration may be based on both glucose and potassium levels, such as to manage glucose homeostasis and kidney disease.

Generally, for patients with CKD, kidney dysfunction affects insulin metabolism by impairing insulin clearance. Because metabolic syndrome and/or diabetes, which are often comorbid with kidney dysfunction, may increase or induce insulin resistance, a higher dose of insulin may be required to reduce glucose levels to the desired level (e.g., healthy range). However, because kidney dysfunction also impairs insulin clearance, which often results in an overabundance of insulin (i.e., prolonged and pronounced insulin), insulin administration may need to be adjusted to avoid hypoglycemia. As such, in certain embodiments, a medication recommendation may include an insulin administration recommendation including a type, dosage, amount, activity rate, activity duration, and/or timing of insulin.

In certain embodiments, an insulin administration recommendation may be to a change in type, dosage, amount, activity rate, and/or activity duration of insulin and/or timing of insulin administration such as to avoid both short-term hyperglycemia and long-term hypoglycemia. Further, the insulin administration recommendation may be individualized per patient to account for differences in insulin resistance across patients based on the patient's glucose clearance rate. As discussed above glucose clearance rates may be indicative of a patient's level of insulin resistance because insulin resistance alters insulin's ability to reduce glucose levels. A patient with a higher insulin resistance may require a different insulin dosage as compared to another patient with a different level of insulin resistance to clear the same amount of glucose. In certain aspects, the individualized insulin dosage recommendation may be modified over time as insulin resistance increases, or decreases, in the patient. For example, an algorithm used by decision support engine 114 may calculate the amount of insulin needed to reduce glucose levels by a value X (e.g., where X is a value greater than zero) based on the user's glucose clearance rate.

In certain embodiments, insulin administration recommendations may be for rapid-acting insulin and determined based on a user's kidney disease progression. For example, in a patient with early-stage kidney disease (e.g., GFR>50 mil/min.), a regular dose of insulin without any adjustments may be recommended. In another example, in a patient with mid stage kidney disease (e.g., 50>GFR>10 mil/min.), a dose reduction (e.g., 25% reduction) may be determined to be optimal and, therefore, recommended. In yet another example, in a patient with ESRD (e.g., mil/min.>GFR), a larger dose reduction (e.g., 50% reduction) may be determined to be optimal and recommended.

In certain embodiments, insulin administration recommendations may be for longer-acting insulin. While, in certain cases, longer-acting insulin may not necessarily be affected by kidney dysfunction, at least not to such an extent that an insulin administration adjustment would be necessary, in other cases, longer-acting insulin may require an insulin administration adjustment. For example, some longer-acting insulin (e.g., glargine and levemir) may require an insulin administration adjustment (e.g., 30% reduction) based on a certain progression of kidney dysfunction (60 mil/min.>GFR) and, therefore, a reduced dose of insulin (e.g., 70% of regular dose) may be recommended.

Further, in certain embodiments, an insulin administration recommendation may include a recommendation for a certain insulin dosage as well as dietary consumption, which may be referred to herein as a combination dosage. For patients with CKD, potassium regulation is important to reduce risk of hyperkalemia and hypokalemia. Because potassium levels are reduced by insulin, the interactions between potassium, insulin, and glucose may be considered in providing insulin administration recommendations. Thus, in a patient with kidney dysfunction who is on insulin, and has increasing potassium levels, an algorithm may be used for the administration of insulin to avoid or treat hyperkalemia while also preventing hypoglycemia. For example, the algorithm may calculate the amount of insulin needed to reduce potassium levels by a value, X (e.g., where X is a value greater than zero), and the amount of glucose and timing for glucose consumption to prevent hypoglycemia. In another example, a combination dose may be recommended to prevent hypokalemia and hypoglycemia.

In certain embodiments, medication recommendations may include adjustments to other glycemic controlling medication to reduce the likelihood of atypical glucose trends. Generally, in addition to insulin metabolism, kidney dysfunction also impairs the metabolism of other glycemic controlling medication. Typically, these other glycemic controlling medications are more effective at reducing glucose levels in kidney dysfunction and preventing hyperglycemia in patients with kidney dysfunction as compared to patients without kidney dysfunction. Therefore, for a patient with kidney dysfunction, adjustments may also be needed for these glycemic controlling medications to avoid hypoglycemia.

In certain embodiments, medication recommendations may include adjustments to type, timing, and/or amount of a glycemic controlling medication. For example, a recommendation may be to take a lower dosage of a glycemic controlling medication. In another example, a recommendation may be to adjust dosage timing (e.g., dose in the morning, before consuming a meal, etc.). In yet another example, a recommendation may be to discontinue use of a glycemic controlling medication. Additionally, alone or in conjunction with other medication recommendations, a recommendation may be to consult with a HCP regarding adjustments to glycemic controlling medication.

Some glycemic controlling medications (e.g., metformin) are associated with a risk of lactic acidosis. Although rare, lactic acidosis is associated with high mortality rates. Additionally, an increased risk of lactic acidosis is associated with kidney dysfunction. Because of this increased risk, a dose reduction is recommended for mild kidney dysfunction (e.g., 45 mL/min./1.73 m2>GFR). Also, metformin contraindicated for patients with severe kidney dysfunction (30 mL/min./1.73 m2>GFR). However, metformin has known renal protective properties. Thus, there is a desire to continue use in patients with kidney dysfunction, if the lactic acidosis risk is reduced. In certain embodiments, based on inputted medication information and lactate levels, decision support engine 114 may determine that a risk for lactic acidosis is increased. In certain embodiments, medication recommendations may be an adjustment to dosage of metformin. For example, a recommendation may be to reduce metformin dosage. In another example, a recommendation may be to cease metformin administration. In yet another example, a recommendation may be to consult with a HCP regarding reduction or cessation of metformin administration.

In certain embodiments, treatment recommendations may include a recommendation for dialysis for the patient. Dialysis is a treatment for kidney failure that rids the body of unwanted toxins, waste products, and excess fluids by filtering a patient's blood. Dialysis helps to keep the potassium, glucose, insulin, phosphorus, and sodium levels in a patient's body balanced. In certain embodiments, dialysis may be recommended based on analyte levels, glucose metrics, glucose clearance rates and/or any other analyte data.

In certain embodiments, diet recommendations may include a recommendation for the patient to consume a fixed amount of glucose at certain intervals (e.g., daily, weekly, etc.). For example, a patient may be recommended to eat a fixed amount of glucose per day based on a level of glucose the patient's kidney is clearing in real-time. Monitoring glucose consumption may help to reduce risk of atypical glucose trends while also ensuring the patient is consuming sufficient glucose to maintain glucose homeostasis.

In certain embodiments, diet recommendations may include a recommendation for the patient to increase their glucose consumption to improve glucose homeostasis. For example, a patient may be recommended to increase glucose consumption where excess insulin, or decreased insulin clearance may result in an atypical glucose trend of hypoglycemia. In certain embodiments, a diet recommendation may be for the timing, and/or amount of glucose to be consumed. For example, a patient may be recommended to consume additional glucose following an insulin dosage to reduce the risk of hypoglycemia. In certain embodiments, diet recommendations may include a recommendation for the patient to reduce their glucose consumption to maintain glucose homeostasis. For example, a patient may be recommended to decrease glucose consumption where inadequate insulin, or insulin resistance may result in an atypical glucose trend of hyperglycemia. In certain embodiments, diet recommendations may be made based on or in conjunction with, medication recommendations. For example, a recommendation may be to consume glucose and administer insulin. In certain embodiments, diet recommendations may be based on or in conjunction with, lifestyle recommendations. For example, a recommendation may be to consume glucose and increase exercise.

In certain embodiments, diet recommendations may include a recommendation for managing kidney disease and glucose homeostasis by recommending that the patient consumes or avoids consuming potassium and/or glucose. For example, a patient may adhere to a low potassium diet and a recommendation to increase glucose consumption may include avoiding consumption of potassium. In certain embodiments, diet recommendations may be based on or in conjunction with medication recommendations. For example, a patient may be recommended to increase potassium consumption, but not glucose consumption, with an insulin administration recommendation to reduce the risk of hyperkalemia and while simultaneously reducing the risk of hyperglycemia.

In certain embodiments, diet recommendations may include recommendations for a patient based on the patient's adherence to a dietary plan to maintain glucose homeostasis. For example, a recommendation may be to reduce glucose consumption to maintain glucose consumption within a set threshold. In certain embodiments, diet recommendations may include recommendations for a patient based on meal type, timing, and/or composition. For example, a recommendation may be for a user to consume a meal now. In another example, a recommendation may be for a user to consume their last meal earlier in the evening (e.g., consume a meal at 5 PM instead of 7 PM). In another example, a recommendation may be for a user to consume a meal with less glucose. In another example, a recommendation may be for a user to consume a meal including complex carbohydrates in order to maintain glucose levels throughout the night. In yet another example, a recommendation may be for user to consume a smaller meal, or skip a meal.

In certain embodiments, lifestyle recommendations (e.g., including exercise recommendations) may include a recommendation for the patient to increase their physical activity to maintain glucose homeostasis. Increased physical activity is one method for reducing glucose levels. In certain embodiments, exercise recommendations may be to engage in physical activity. In certain embodiments, exercise recommendations may be given with other recommendations (e.g., diet and/or treatment recommendations). In certain embodiments, one or more models may determine when such patients should engage in physical activity based on glucose levels, kidney function, and/or insulin levels for the patient. Recommendations may include a modified physical activity schedule, and/or additional rest breaks. In certain embodiments, other analyte data or non-analyte data, such as heart rate or respiratory rate data, may be used in combination with glucose data to provide such exercise recommendations. For example, glucose data, potassium data, lactate data, and heart rate may indicate a user should take a rest break to maintain glucose homeostasis and manage kidney disease. In another example, these analyte or non-analyte data may be used to guide specific exercise recommendations such as the intensity of exercise and the duration, to optimize glucose levels.

In certain embodiments, medical intervention recommendations may include a recommendation for the patient to seek medical aid. For example, in certain embodiments, the medical intervention recommendation may indicate to a patient, that the patient needs to immediately go to the emergency room and/or contact their physician. In certain other embodiments, the medical intervention recommendation may automatically alert the HCP of the patient as to the condition of the patient for intervention by the HCP. In certain other embodiments, the medical intervention recommendation may alert medical personnel to send aid to the patient, e.g., trigger ambulance services or paramedic services to provide urgent pre-hospital treatment and stabilization to the patient and/or transport of the patient to definitive care. In certain embodiments, decision support engine 114 may make a medical intervention recommendation based on a patient's ability to seek medical help and/or the accessibility of the patient to medical help.

In certain embodiments, medical intervention recommendations may be based on, for example, blood pressure levels. Blood pressure monitoring over time may be used to determine if the patient is developing hypertension. For example, if the patient's average blood pressure increases over a time period (e.g., one month or two months), a medical intervention recommendation may be to seek medication attention and/or contact their physician. In certain embodiments, decision support engine 114 and/or the patient's physician may recommend the patient begin a RAASi medication and/or complete a light exercise following meals to reduce glucose levels and prevent the development and/or progression of hypertension.

In certain embodiments, the atypical glucose trend generated at block 506 may be used to determine the patient's risk of hospital admission/readmission. In certain embodiments, the atypical glucose trend generated at block 506 may be used to better understand post hospital discharge stability of the patient when determining whether to discharge the patient. In certain embodiments, the atypical glucose trend generated at block 506 may be used to determine the level of care a patient should receive upon hospital admittance. For example, a patient admitted to a hospital may be one of many patients in the hospital. Accordingly, in certain aspects, the atypical glucose trend generated for the patient may be compared to similarly generated atypical glucose trends of other patients in the hospital to better inform medical personnel in the hospital where the patient ranks among other patients, in terms of the level of care needed, as well as the urgency of attention needed by the patient among other patients. This may be especially important where a patient is at high risk of experiencing an acute deadly event within a small amount of time after being admitted into the hospital (e.g., as opposed to waiting four, or more, hours prior to receiving assistance or care by medical personnel).

As discussed herein, machine learning models deployed by decision support engine 114 include one or more models trained by training server system 140, as illustrated in FIG. 1, to provide various types of predictions, as discussed in relation to FIGS. 4 and 5. FIG. 6 describes in further detail techniques for training one or more machine learning models for generating predictions associated with diabetes in patients with kidney disease, according to certain embodiments of the present disclosure. Predictions associated with diabetes may include (1) predictions as to the presence and/or severity of diabetes in patients with kidney disease (e.g., a user illustrated in FIG. 1), (2) identify risk of hyperglycemia and/or hypoglycemia, and/or (3) predictions as to optimal treatment for a patient with diabetes and kidney disease.

Method 600 begins, at block 602, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more patients who are not, or were not previously, users of continuous analyte monitoring system 104 and/or application 106. In certain embodiments, historical records database 112 may include one or more data sets of historical patients with (1) no diabetes and no kidney disease, (2) no diabetes and varying stages of kidney disease, (3) varying stages of diabetes and no kidney disease, or (4) varying stages of diabetes and varying stages of kidney disease.

Retrieval of data from historical records database 112 by training server system 140, at block 602, may include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository.

As an illustrative example, at block 602, training server system 140 may retrieve information for 100,000 patients with varying stages of diabetes and/or varying stages of kidney disease stored in historical records database 112 to train a model to predict the presence, type, and/or severity of diabetes in a user. Each of the 100,000 patients may have a corresponding data record (e.g., based on their corresponding user profile)), stored in historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by training server system 140), resulting in features that can be used as input features for training the ML model. For example, a patient record may include or be used to generate features related to an age of a patient, a gender of the patient, an occupation of the patient, analyte levels for the patient over time, analyte level rates of change and/or trends for the patient over time, physiological parameters associated with different kidney disease and/or diabetes stages for the patient over time, and/or any information provided by inputs 128 and/or metrics 130, etc. Features used to train the machine learning model(s) may vary in different embodiments.

In certain embodiments, each historical patient record retrieved from historical records database 112 is further associated with a label indicating whether the patient was healthy or experienced some variation of kidney disease and/or diabetes, a previously determined kidney disease and/or diabetes diagnosis and/or stage of chronic kidney disease (CKD) for the patient, a kidney disease risk assessment, a diabetes risk assessment, treatment(s), and/or similar metrics. What the record is labeled with would depend on what the model is being trained to predict.

At block 604, method 600 continues by training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output may indicate a kidney disease diagnosis, a diabetes diagnosis, a diabetes severity, a diabetes type, a risk assessment associated with a current diabetes diagnosis, reduced glucose homeostasis, and/or the level of risk the patient was at for experiencing hyperglycemia/hypoglycemia, a likelihood of an atypical glucose trend, a decision support recommendation (e.g., treatment recommendation, diet recommendation, exercise recommendation, etc.). In certain embodiments, the output may indicate whether a recommended treatment is effective at reducing risk (e.g., associated with an atypical glucose trend).

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict the presence, type, and/or severity of diabetes (or its recommended treatments) more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 606, training server system 140 deploys the trained model(s) to make predictions associated with diabetes during runtime. In some embodiments, this includes transmitting some indication of the trained model(s) (e.g., a weights vector) that can be used to instantiate the model(s) on another device. For example, training server system 140 may transmit the weights of the trained model(s) to decision support engine 114. The model(s) can then be used to assess, in real-time, the presence and/or severity of diabetes of a user using application 106, provide treatment recommendations, and/or make other types of predictions discussed above. In certain embodiments, the training server system 140 may continue to train the model(s) in an “online” manner by using input features and labels associated with new patient records.

Further, similar methods for training illustrated in FIG. 6 using historical patient records may also be used to train models using patient-specific records to create more personalized models for making predictions associated with atypical glucose trends for patients with kidney dysfunction. In particular, a model trained using historical patient records that is deployed for a particular user, may be further re-trained after deployment. For example, the model may be re-trained after the model is deployed for a specific patient to create a more personalized model for the patient. The more personalized model may be able to more accurately predict atypical glucose trends for the patient based on the patient's own data (as opposed to only historical patient record data), including the patient's own glucose metrics.

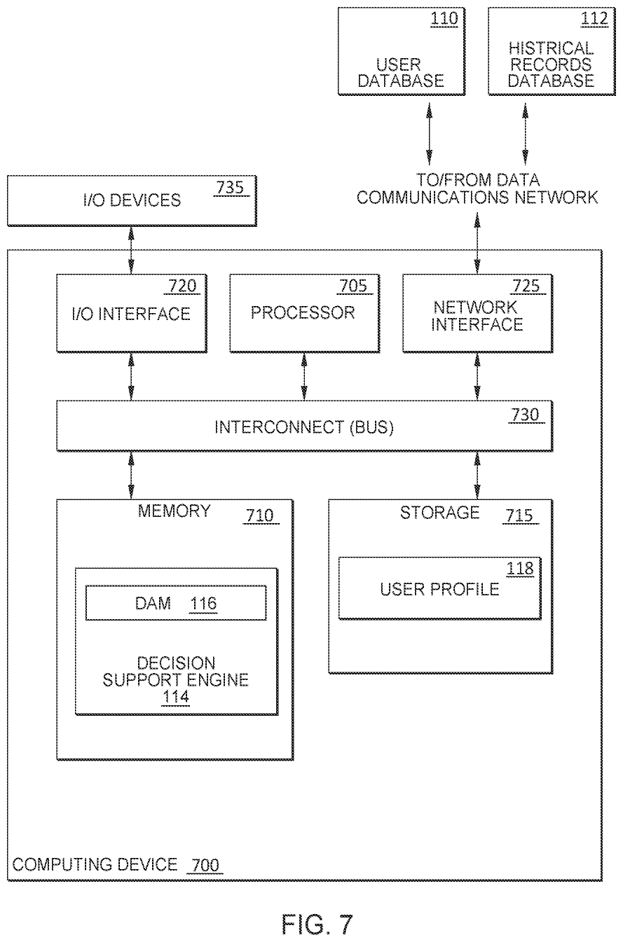

FIG. 7 is a block diagram depicting a computing device 700 configured for (1) predictions as to the presence and/or severity of diabetes in patients with kidney disease (e.g., a user illustrated in FIG. 1), (2) identify risk of hyperglycemia and/or hypoglycemia; and/or (3) predictions as to optimal treatment for a patient, according to certain embodiments disclosed herein. Although depicted as a single physical device, in embodiments, computing device 700 may be implemented using virtual device(s), and/or across a number of devices, such as in a cloud environment. As illustrated, computing device 700 includes a processor 705, memory 710, storage 715, a network interface 725, and one or more I/O interfaces 720. In the illustrated embodiment, processor 705 retrieves and executes programming instructions stored in memory 710, as well as stores and retrieves application data residing in storage 715. Processor 705 is generally representative of a single CPU and/or GPU, multiple CPUs and/or GPUs, a single CPU and/or GPU having multiple processing cores, and the like. Memory 710 is generally included to be representative of a random access memory (RAM). Storage 715 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 735 (such as keyboards, monitors, etc.) can be connected via the I/O interface(s) 720. Further, via network interface 725, computing device 700 can be communicatively coupled with one or more other devices and components, such as user database 110 and/or historical records database 112. In certain embodiments, computing device 700 is communicatively coupled with other devices via a network, which may include the Internet, local network(s), and the like. The network may include wired connections, wireless connections, or a combination of wired and wireless connections. As illustrated, processor 705, memory 710, storage 715, network interface(s) 725, and I/O interface(s) 720 are communicatively coupled by one or more interconnects 730. In certain embodiments, computing device 700 is representative of display device 107 associated with the user. In certain embodiments, as discussed above, display device 107 can include the user's laptop, computer, smartphone, and the like. In another embodiment, computing device 700 is a server executing in a cloud environment.

In the illustrated embodiment, storage 715 includes user profile 118. Memory 710 includes decision support engine 114, which itself includes DAM 116. Decision support engine 114 is executed by computing device 700 to perform operations in workflow 400 of FIG. 4, operations of method 500 in FIG. 5, and/or operations of method 600 in FIG. 6.

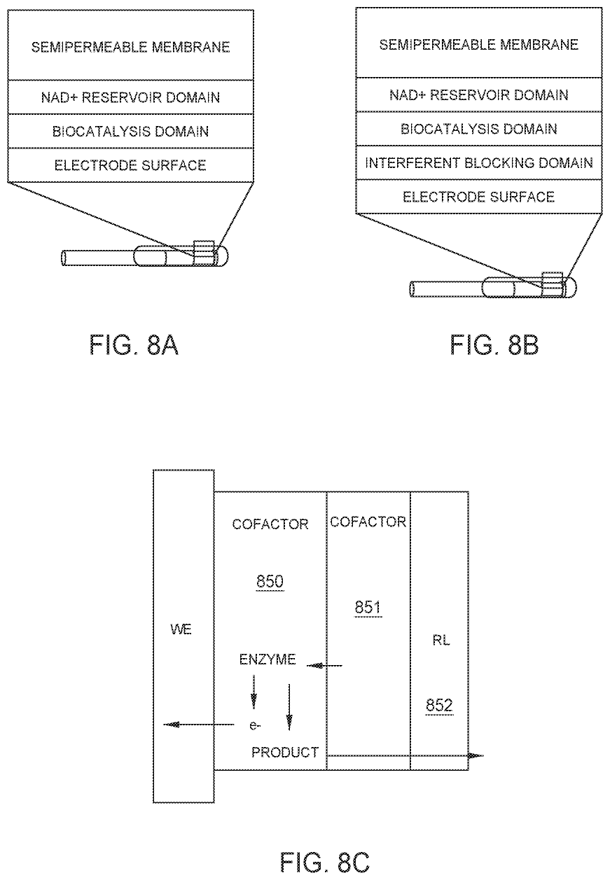

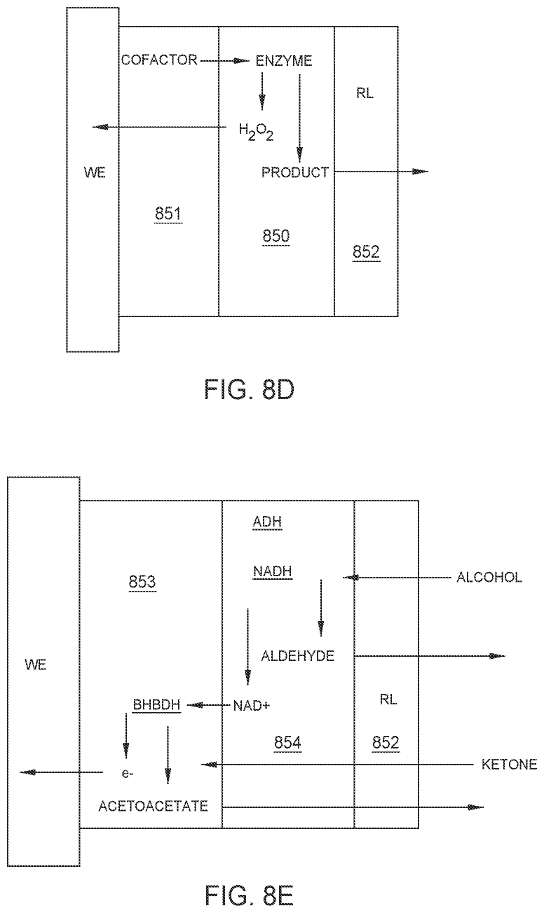

As described above, continuous analyte monitoring system 104, described in relation to FIG. 1, may be a multi-analyte sensor system including a multi-analyte sensor. FIGS. 8-12 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galacto se/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)±/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)±/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 8A. With reference to FIG. 8B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 8A-8B, one or more of the membranes provides a NAD+ reservoir domain providing a reservoir for NAD+. In one example, one or more interferent blocking membranes is used, and potentiostat is utilized to measure H₂O₂ production or 02 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Another example of a continuous ketone analyte detection configuration employing electrode-associated mediator-coupled diaphorase/NAD+/dehydrogenase is depicted below:

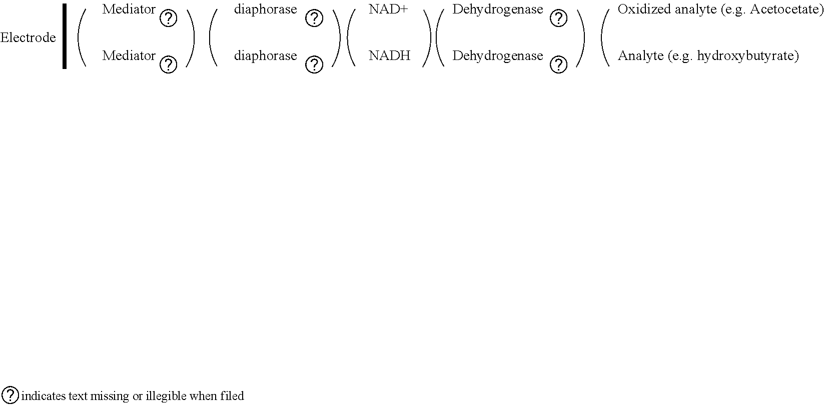

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 8A-8B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxy steroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 8C depicts this exemplary configuration, of an enzyme domain 850 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 851 comprising an amount of cofactor is positioned adjacent the first enzyme domain. The amount of cofactor in the second membrane can provide an excess for the enzyme, e.g., to extend sensor life. One or more resistance domains 852 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 8D depicts an alternative enzyme domain configuration comprising a first membrane 851 with an amount of cofactor that is positioned more proximal to at least a portion of a WE surface. Enzyme domain 850 comprising an amount of enzyme is positioned adjacent the first membrane.

In the membrane configurations depicted in FIGS. 8C-8D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg′. One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 8E depicts another continuous multi-analyte membrane configuration, where {beta}-hydroxybutyrate dehydrogenase BHBDH in a first enzyme domain 853 is positioned proximate to a working electrode WE and second enzyme domain 854, for example, comprising alcohol dehydrogenase (ADH) and NADH is positioned adjacent the first enzyme domain. One or more resistance domains RL 852 may be deployed adjacent to the second enzyme domain 854. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

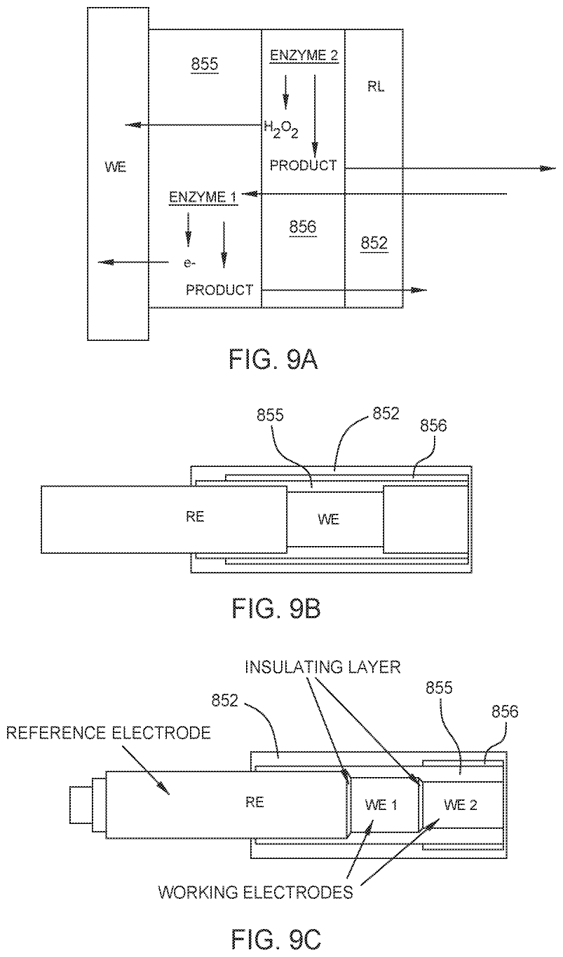

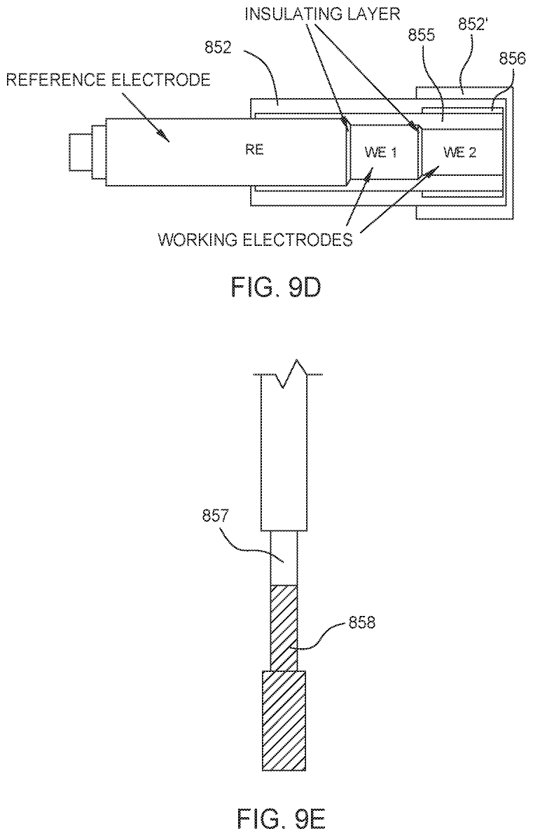

In one example, at least a dual enzyme domain configuration in which each layer contains one or more specific enzymes and optionally one or more cofactors is provided. In a broad sense, one example of a continuous multi-analyte sensor configuration is depicted in FIG. 9A where a first membrane 855 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 856 (EZL2) with at least one second enzyme (Enzyme 2) is positioned adjacent 855 ELZ1, and is generally more distal from WE than EZL1. One or more resistance domains (RL) 852 can be provided adjacent EZL2 856, and/or between EZL1 855 and EZL2 856. The different enzymes catalyze the transformation of the same analyte, but at least one enzyme in EZL2 856 provides hydrogen peroxide and the other at least one enzyme in EZL1 855 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 9A, a first analyte diffuses through RL 852 and into EZL2 856 resulting in peroxide via interaction with Enzyme 2. Peroxide diffuses at least through EZL1 855 to WE and transduces a signal that corresponds directly or indirectly to the first analyte concentration. A second analyte, which is different from the first analyte, diffuses through RL 852 and EZL2 856 and interacts with Enzyme 1, which results in electron transfer to WE and transduces a signal that corresponds directly or indirectly to the second analyte concentration.

As shown in FIG. 9B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 856 providing hydrogen peroxide and the at least other enzyme in EZL1 855 not providing hydrogen peroxide, e.g., providing electron transfer to the WE surface corresponding directly or indirectly to a concentration of the analyte.

In one example, an inner layer of the at least two enzyme domains EZL1, EZL2 855, 856 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 855 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 855 is directly adjacent the WE.

The second layer of at least dual enzyme domain (the outer layer EZL2 856) of FIG. 9B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 856 and through the inner layer EZL1 855 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 855, 856) associated with each electrode. The two (or more) signals can be broken down into components to detect the individual signal and signal artifacts generated by each of EZL1 855 and EZL2 856 in response to the detection of two analytes. In some examples, each EZL detects a different analyte. In other examples, both EZLs detect the same analyte.

In another alternative exemplary configuration, as shown in FIGS. 9C-9D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2), In one example, the first analyte detected by WE1 is glucose, and the second analyte detected by WE2 is lactate. In another example, the first analyte detected by WE1 is glucose, and the second analyte detected by WE2 is ketones.

Thus, FIGS. 9C-9D depict exemplary configurations of a continuous multi-analyte sensor construct in which EZL1 855, EZL2 856 and RL 852 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 9C-9D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 9C that covers the reference electrode and WE1, WE2. An addition resistance domain is provided in the configuration of FIG. 9D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H₂O₂ producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 9C-9D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 9D, such an arrangement of RL's is depicted, where an additional RL 852′ is adjacent WES2 but substantially absent from WES1.

In one example of measuring two different analytes, the above configuration comprising enzyme domain EZL1 855 comprising one or more enzyme(s) and one or more mediators for at least one enzyme of EZL1 to provide for direct electron transfer to the WES1 and determining a concentration of at least a first analyte. In addition, enzyme domain EZL2 856 can comprise at least one enzyme that provides peroxide (e.g., hydrogen peroxide) or consumes oxygen during catalysis with its substrate. The peroxide or the oxygen produced in EZL2 856 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 856. The combinations of electrode material and enzyme(s) as disclosed herein are examples and non-limiting.

In one example, the potentials of P1 and P2 can be separated by an amount of potential so that both signals (from direct electron transfer from EZL1 855 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 855, EZ2 856 were associated with different WEs, e.g., platinum WE2, and gold WE1 was prepared. In this exemplary case, EZL1 855 contained glucose oxidase and a mediator coupled to WE1 to facilitate electron direct transfer upon catalysis of glucose, and EZL2 856 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 9E, an example of such composite electrode surfaces is shown, in which an extended platinum covered wire 857 is half coated with carbon 858, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE 1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 855) and glucose sensing (glucose oxidase in EZL2 856). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

Glycerol Sensor Configurations

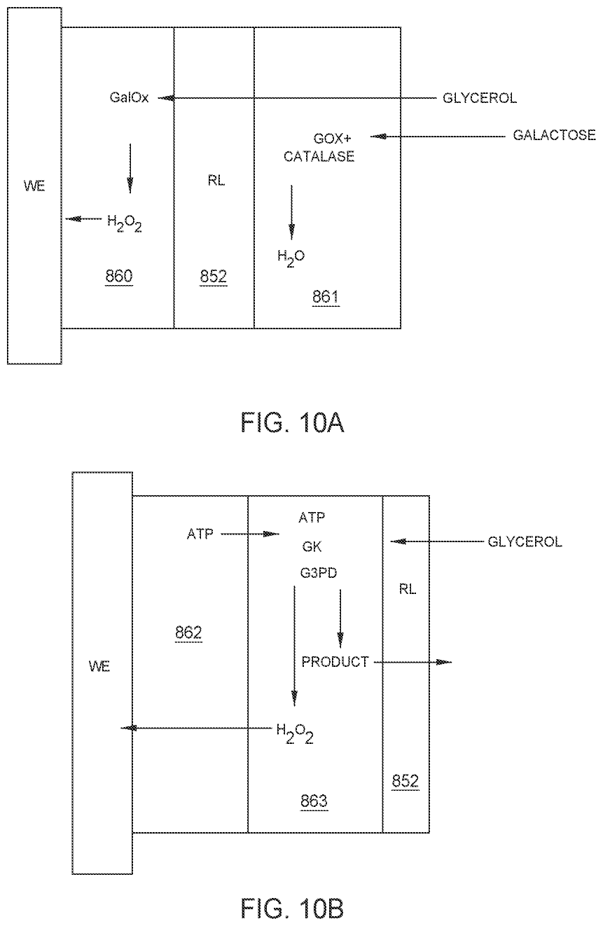

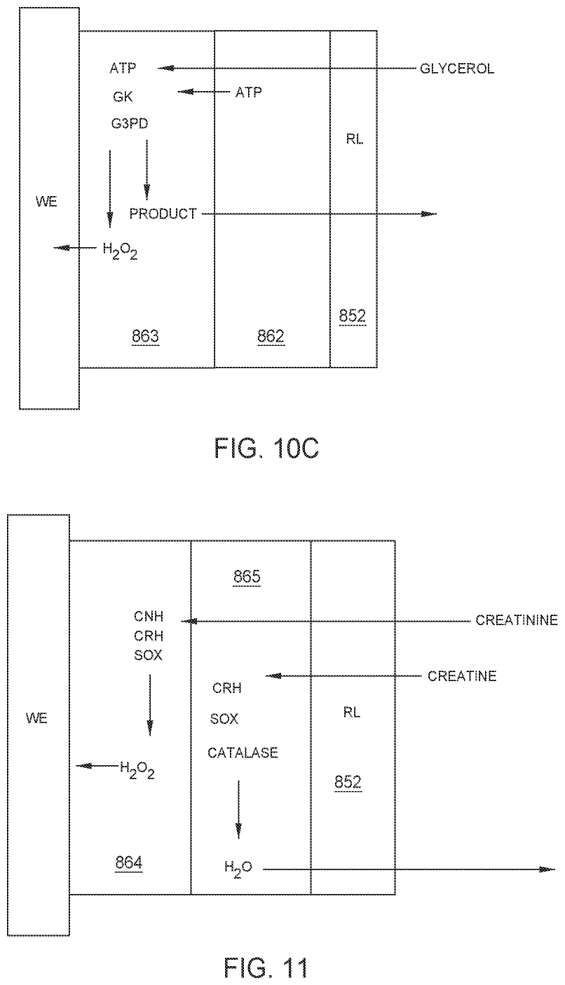

As shown in FIG. 10A, an exemplary continuous glycerol sensor configuration is depicted where a first enzyme domain EZL1 860 comprising galactose oxidase is positioned proximal to at least a portion of a WE surface. A second enzyme domain EZL2 861 comprising glucose oxidase and catalase is positioned more distal from the WE. As shown in FIG. 10A, one or more resistance domains (RL) 852 are positioned between EZL1 860 and EZL2 861. Additional RLs can be employed, for example, adjacent to EZL2 861. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 860 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 860 and EZL2 861 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 861 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 852. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 10B and 10C, exemplary sensor configurations are depicted where in one example (FIG. 10B), one or more cofactors (e.g. ATP) 862 is proximal to at least a portion of an WE surface. One or more enzyme domains 863 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 862. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 10C, where one or more enzyme domains 863 comprising G3PD, at least one cofactor and at least one regenerating enzyme, are positioned proximal to at least a portion of WE surface, with one or more cofactor reservoirs 862 adjacent to the enzyme domains comprising G3PD and more distal from the WE surface, and one or more RL's 852 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (PDX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 11 depicts an exemplary continuous sensor configuration for creatinine. In the example of FIG. 11, the sensor includes a first enzyme domain 864 comprising CNH, CRH, and SOX are adjacent a working electrode WE, e.g., platinum. A second enzyme domain 865 is positioned adjacent the first enzyme domain and is more distal from the WE. One or more resistance domains (RL) 852 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

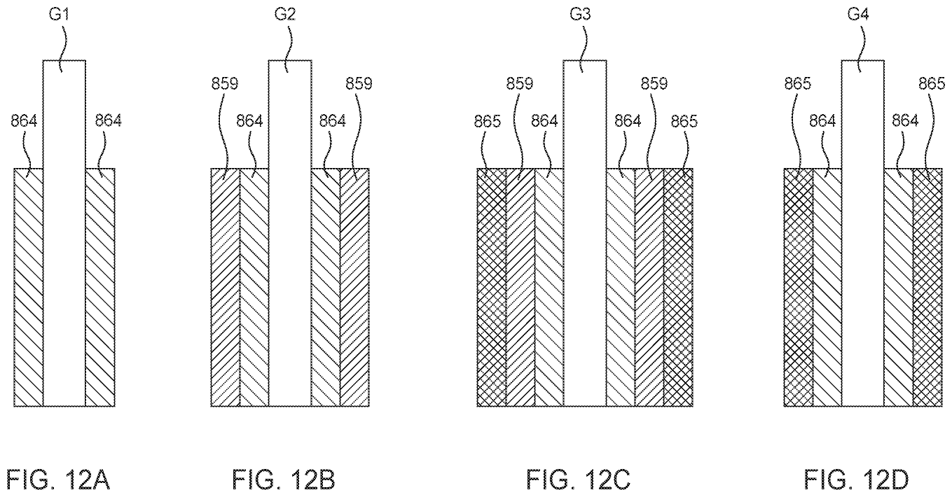

FIG. 12A-12D depict alternative continuous lactose sensor configurations. Thus, in an enzyme domain EZL1 864 most proximal to WE (G1), comprising GalOx and lactase, provides a lactose sensor that is sensitive to galactose and lactose concentration changes and is essentially non-transducing of glucose concentration. As shown in FIGS. 12B-12D, additional layers, including non-enzyme containing layers 859, and an enzyme domain 865 (e.g., a lactase enzyme containing layer), and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

In certain embodiments, continuous analyte monitoring system 104 may be a potassium sensor, as discussed in reference to FIG. 1. FIGS. 8-15 describe an example sensor device used to measure an electrophysiological signal and/or concentration of a target analyte (e.g., potassium), according to certain embodiments of the present disclosure.

The term “ion” as used herein is a broad term, and is to be given its ordinary and customary meaning to a person of ordinary skill in the art (and is not to be limited to a special or customized meaning), and refers without limitation to an atom or molecule with a net electric charge due to the loss or gain of one or more electrons. Ions in a biological fluid may be referred to as “electrolytes.” Nonlimiting examples of ions in biological fluids include sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). An ion is an example of an analyte.

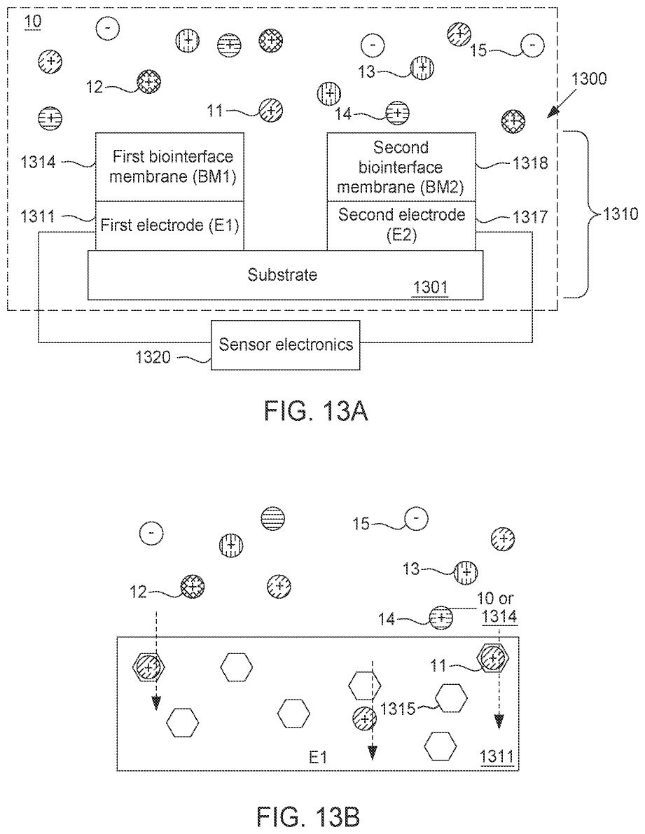

FIG. 13A schematically illustrates an example configuration and component of a device 1300 for measuring an electrophysiological signal and/or concentration of a target analyte such as a target ion 11 in a biological fluid 10 in vivo. Turning first to FIG. 13, device 1300 includes indwelling sensor 1310 and sensor electronics 1320. Sensor 1310 includes substrate 1301, first electrode (E1) 1311 disposed on the substrate, and a second electrode (E2) 1317 disposed on the substrate. First electrode 1311 may be referred to as a working electrode (WE), while second electrode 1317 may be referred to as a reference electrode (RE). The sensor electronics 1320 may be configured to generate a signal corresponding to an electromotive force (EMF). In some examples, the EMF is at least partially based on a potential difference that is generated between the first electrode 1311 and the second electrode 1317 responsive to biological fluid 10 conducting the electrophysiological signal to first electrode 1311. Sensor electronics 1320 may be configured to use the signal to generate an output corresponding to a measurement of the signal. In various examples, the EMF is at least partially based on a potential difference between (i) either the first electrode 1311 or the second electrode 1317 and (ii) another electrode which is spaced apart from the first electrode or second electrode.

Additionally, or alternatively, in some examples, device 1300 may include an ionophore, such as ionophore 1315 as shown in FIG. 13B, disposed on the substrate 1301 and configured to selectively transport the target ion 11 to or within the first electrode 1311. The EMF may be at least partially based on a potential difference may be generated between the first electrode 1311 and the second electrode 1317 responsive to the ionophore transporting the target ion to or through the first electrode 1311. The sensor electronics 1320 (and/or an external device that receives the signal via a suitable wired or wireless connection) may be configured to use the signal to generate an output corresponding to a measurement of the concentration of the target ion in the biological fluid. Further details regarding the configuration and use of sensor electronics 1320 are provided further below.

Optionally, the first electrode 1311 may be used to measure an electrophysiological signal in addition to ion concentration. In other examples, such as when device 1300 is configured to detect an electrophysiological signal but not an ion concentration, first electrode 1311 need not include an ionophore, such as ionophore 1315 as shown in FIG. 13B. In other examples, the first electrode 1311 may include an ionophore that is inactive such that it does not interfere with the measurement of the electrophysiological signal.

In a manner such as illustrated in FIG. 13A, biological fluid 10 may include a plurality of ions 11, 12, 13, 14, and 15. Device 1300 may be configured to measure the concentration of ion 11, and accordingly such ion may be referred to as a “target” ion. Target ion 11 may be any suitable ion, and in nonlimiting examples is selected from the group consisting of sodium (Nat), potassium (K⁺), magnesium (Mg²⁺), calcium (Ca²⁺), hydrogen (H⁺), lithium (Li⁺), chloride (Cl⁻), sulfite (SO₃²⁻), sulfate (SO₄²⁻), phosphate (PO₄³⁻), and ammonium (NH₄⁺). Ions 12, 13, 14, and 15 may be others of the group consisting of sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). Ions 12, 13, 14, and 15 may be considered interferants to the measurement of target ion 11 because they have the potential interfere with the measurement of target ion 11 by sensor to produce a signal that does not accurately represent the concentration of target ion 11. Ionophore, such as ionophore 1315 as shown in FIG. 13B, may be selected so as to selectively transport target ion 11 to or within first electrode 1311 and to inhibit, fully, partially and/or substantially, the transport of one or more of ions 12, 13, 14, or 15 to or within first electrode 1311. For example, as illustrated in FIG. 13B, ionophore 1315 may selectively transport, or selectively bind, target ions 11 from biological fluid 10 or from biointerface membrane 1314 (if provided, e.g., as described below) to and within first electrode 1311, while ions 12, 13, 14, and 15 may substantially remain within biological fluid 10 or biointerface membrane 1314. Accordingly, contributions to the potential difference between first electrode 1311 and second electrode 1317 responsive to the transport of ions to or within first electrode 1311 substantially may be primarily caused by target ion 11 instead of by one or more of ions 12, 13, 14, or 15.

A wide variety of ionophores 1315 may be used to selectively transport corresponding ions in a manner such as described with reference to FIGS. 13A-13B. For example, where the target ion 11 is hydrogen (via peroxide), the ionophore 1315 may be tridodecylamine, 4-nonadecylpyridine, N,N-dioctadecylmethylamine, octadecyl isonicotinate, calix[4]-aza-crown. Or, for example, where the target ion 11 is lithium, the ionophore 1315 may be ETH 149, N,N,N′,N′,N″,N″-hexacyclohexyl-4,4′,4″-propylidynetris(3-oxabutyramide), or 6,6-Dibenzyl-1,4,8-11-tetraoxacyclotetradecane. Or, for example, where the target ion 11 is sulfite, the ionophore 1315 may be octadecyl 4-formylbenzoate. Or, for example, where the target ion 11 is sulfate, the ionophore 1315 may be 1,3-[bis(3-phenylthioureidomethyl)]benzene or zinc phthalocyanine. Or, for example, where the target ion 11 is phosphate, the ionophore 1315 may be 9-decyl-1,4,7-triazacyclodecane-8,10-dione. Or, for example, where the target ion 11 is sodium, the ionophore 1315 may be 4-tert-butylcalix[4]arene-tetraacetic acid tetraethyl ester (sodium ionophore X) or calix[4]arene-25,26,27,28-tetrol (calix[4]arene). Or, for example, where the target ion 11 is potassium, the ionophore 1315 may be potassium ionophore II (BB15C5) or valinomycin. Or, for example, where the target ion 11 is magnesium, the ionophore 1315 may be 4,5-bis(benzo ylthio)-1,3-dithiole-2-thione (Bz2dmit) or 1,3,5-Tris[10-(1-adamantyl)-7,9-dioxo-6,10-diazaundecyl]benzene (magnesium ionophore VI). Or, for example, where the target ion 11 is calcium, the ionophore 1315 may be calcium ionophore I (ETH 1001) or calcium ionophore II (ETH129). Or, for example, where the target ion 11 is chloride, the ionophore 1315 may be tridodecylmethylammonium chloride (TDMAC). Or, for example, where the target ion 11 is ammonium, the ionophore 1315 may be nonactin.

In the nonlimiting example illustrated in FIG. 13A, ionophore 1315 may be provided within first electrode 1311, and in such example the first electrode may be referred to as an ion-selective electrode (ISE), since the ionophore 1315 selectively transports the target ion 11. In some examples, first electrode 1311 may include a conductive polymer optionally having ionophore 1315 therein. Illustratively, the conductive polymer may be present in an amount of about 90 to about 99.5 weight percent in the first electrode 1311. The ionophore 1315 may be present in an amount of about 0.5 to about 10 weight percent in the first electrode. In some examples, the conductive polymer may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT).

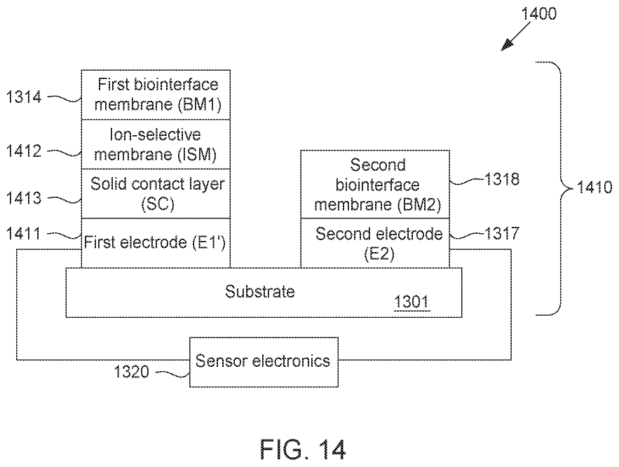

While conductive polymers (such as listed above) suitably may be used in a first electrode 1311 that excludes ionophore 1315, other materials alternatively may be used, some nonlimiting examples of which are described below with reference to FIG. 14. Optionally, ionophore 1315 may be provided in a membrane which is disposed on a first electrode 1311 (which electrode may exclude ionophore 1315), e.g., such as will be described below with reference to FIG. 14.

First electrode 1311 may be configured in such a manner as to enhance its biocompatibility. For example, first electrode 1311 may substantially exclude any plasticizer, which otherwise may leach into the biological fluid 10, potentially causing toxicity and/or a degradation in device performance. As used herein, the “substantial” exclusion of materials such as plasticizers is intended to mean that the first electrode 1311 or other aspects discussed herein do not contain detectable quantities of the “substantially” excluded material. In some examples, the first electrode 1311 may consist essentially of the conductive polymer, optionally in addition to the ionophore 1315. In some examples, the first electrode 1311 may consist essentially of the conductive polymer, the ionophore 1315, and an additive with ion exchanger capability. Such an additive may act as an ion exchanger. In one example, the additive contributes to the ion selectivity. In another example, the additive may not provide ion selectivity. For example, the additive may help to provide a substantially even concentration of the ion in the membrane. Additionally, or alternatively, the additive may help any change in ion concentration in the biofluid to cause an ion exchange within the membrane that may induce a non-selective potential difference. Additionally, or alternatively, the ionophore and the ion exchanger may form a complex which improves the ionophore's selectivity towards the target ion as compared to the selectivity of the ionophore alone.

Optionally, the additive may include a lipophilic salt. In nonlimiting examples, the lipophilic salt is selected from the group consisting of sodium tetrakis[3,5-bis(trifluoromethyl)phenyl]borate (NaTPFB), sodium tetraphenylborate (NaTPB), potassium tetrakis [3,5-bis(trifluoromethyl)phenyl]borate (KTFPB), and potassium tetrakis(4-chlorophenyl)borate (KTC1PB). The additive may be present in an amount of about 0.01 to about 1 weight percent in the first electrode, or other suitable amount.

Other materials within sensor 1310 may be selected. For example, substrate 1301 may include a material selected from the group consisting of: metal, glass, transparent conductive oxide, semiconductor, dielectric, ceramic, and polymer (such as biopolymer or synthetic polymer). In some examples, second electrode 1317 may include a metal, a metal alloy, a transition metal oxide, a transparent conductive oxide, a carbon material, a doped semiconductor, a binary semiconductor, a ternary semiconductor, or a conductive polymer. The binary semiconductor may include any two elements suitable for use in a semiconductor. The ternary semiconductor may include two or more binary semiconductors. In examples where a metal or a metal alloy is used, the metal or metals used can be selected from the group consisting of: gold, platinum, silver, iridium, rhodium, ruthenium, nickel, chromium, and titanium. The metal optionally may be oxidized or optionally may be in the form of a metal salt. A nonlimiting example of an oxidized metal which may be used in second electrode 1317 is iridium oxide. The carbon material may be selected from the group consisting of: carbon paste, graphene oxide, carbon nanotubes, C60, porous carbon nanomaterial, mesoporous carbon, glassy carbon, hybrid carbon nanomaterial, graphite, and doped diamond. The doped semiconductor may be selected from the group consisting of: silicon, germanium, silicon-germanium, zinc oxide, gallium arsenide, indium phosphide, gallium nitride, cadmium telluride, indium gallium arsenide, and aluminum arsenide. The transition metal oxide may be selected from the group of: titanium dioxide (TiO₂), iridium dioxide (IrO₂), platinum dioxide (PtO₂), zinc oxide (ZnO), copper oxide (CuO), cerium dioxide (CeO₂), ruthenium(IV) oxide (RuO₂), tantalum pentoxide (Ta₂O₅), titanium dioxide (TiO₂), molybdenum dioxide (MoO₂), and manganese dioxide (MnO₂). The metal alloy may be selected from the group consisting of: platinum-iridium (Pt—Ir), platinum-silver (Pt—Ag), platinum-gold (Pt—Au), gold-iridium (Au—Ir), gold-copper (Au—Cu), gold-silver (Au—Ag), and cobalt-iron (Co—Fe).

The conductive polymer that may be used for the sensor 2010 may be selected from the group consisting of: poly(3,4-ethylenedioxythiophene) (PEDOT), poly(3,4-ethylenedioxythiophene) polystyrene sulfonate (PEDOT:PSS), polyaniline (PANI), poly(pyrrole) (PPy), or poly(3-octylthiophene) (POT). That is, first electrode 1311 and second electrode 1317 optionally may be formed of the same material as one another, or may be formed using different materials than one another. In the nonlimiting example illustrated in FIG. 13A, first electrode 1311 and second electrode 1317 may be disposed directly on substrate 1301, or alternatively may be disposed on substrate 1301 via one or more intervening layers (not illustrated).

The biocompatibility of sensor 1310 optionally may be further enhanced by providing a biointerface membrane over one or more component(s) of sensor 1310. For example, in the nonlimiting configuration illustrated in FIG. 13A, a first biointerface membrane (BM1) 1314 may be disposed on the ionophore 1315 and the first electrode 1311. In another example, the first biointerface membrane (BM1) 1314 may be disposed on the ionophore 1315 and the first electrode 1311, and a second biointerface membrane (BM2) 1318 may be disposed on the second electrode 1317. Although FIG. 13A may suggest that the biointerface membrane(s) have a rectangular shape for simplicity of illustration, it should be apparent that the membrane(s) may conform to the shape of any underlying layers. In some examples, the biointerface membrane(s) may be configured to inhibit biofouling of the ionophore 1315, the first electrode 1311, and/or the second electrode 1317. Nonlimiting examples of materials which may be included in the biointerface membrane(s) include hard segments and/or soft segments. Examples of hard and soft segments used for the biointerface membrane 1314/1314′/1318 or other biointerface membranes as discussed herein include aromatic polyurethane hard segments with Si groups, aliphatic hard segments, polycarbonate soft segments or any combination thereof. In other examples of biointerface membrane(s) such as 1314/1314′/1318 or other biointerface membranes discussed herein, PVP may not be included. In this example where no PVP is included, the biointerface membrane (1318, 1314, 1314′, or other biointerface membranes as discussed herein) may include polyurethane and PDMS. In some examples, which may be combined with other examples herein, the biointerface membranes discussed herein may include one or more zwitterionic compounds.

Whereas ionophore 1315 is included within first electrode 1311 in the example described with reference to FIG. 13A, in the example illustrated in FIG. 14 first electrode 1411 does not include ionophore 1315 (and thus may be referred to as E1′ rather than E1). Instead, ionophore 1315 may be within an ion-selective membrane (ISM) 1412 disposed on the first electrode 1411. Ionophores 1315 may selectively transport target ion 11 to first electrode 1411 in a manner similar to that described with reference to FIGS. 13A-13B, and such transport may cause a potential difference between the first electrode 1411 and second electrode 1317 based upon which sensor electronics 1320 may generate an output corresponding to a measurement of the concentration of target ion 11 in biological fluid 10. It will be appreciated that in examples in which device 1300 is used to measure an electrophysiological signal and is not used to measure an ion concentration, ISM 1412 may be omitted.

In a manner similar to that described with reference to first electrode 1311, ion-selective membrane 1412 substantially may exclude any plasticizer. In some examples, ion-selective membrane 1412 may consist essentially of a biocompatible polymer and ionophore 1315 configured to selectively bind the target ion. Alternatively, in some examples, the ion-selective membrane 1412 may consist essentially of a biocompatible polymer, an ionophore 1315 configured to selectively bind the target ion 11, and an additive with ion exchanger capability, such as a lipophilic salt. Nonlimiting examples of lipophilic salts, and nonlimiting amounts of additives, biocompatible polymers, and ionophores are provided above with reference to FIGS. 13A-13B. Whereas first electrode 1311 includes a conductive polymer so as to be able to provide ionophore 1315 therein while retaining the electrical conductivity of an electrode, additional types of materials may be used in ion-selective membrane 1412 because the ion-selective membrane 1412 need not be used as an electrode. For example, the biocompatible polymer of the ion-selective membrane 1412 may include a hydrophobic polymer. Illustratively, the hydrophobic polymer may be selected from the group consisting of silicone, fluorosilicone (FS), polyurethane, polyurethaneurea, polyurea. In one example, the biocompatible polymer of the ISM 1412 (or other ion-selective membranes or other membranes discussed here) may include one or more block copolymers, which may be segmented block copolymers. In one example, the hydrophobic polymer may be a segmented block copolymer comprising polyurethane and/or polyurea segments, and/or polyester segments, and one or more of polycarbonate, polydimethylsiloxane (PDMS), methylene diphenyl diisocyanate (MDI), polysulfone (PSF), methyl methacrylate (MMA), poly(ε-caprolactone) (PCL), and 1,4-butanediol (BD). In other examples, the hydrophobic polymer may alternately or additionally include poly(vinyl chloride) (PVC), fluoropolymer, polyacrylate, and/or polymethacrylate.

In one example, the biocompatible polymer may include a hydrophilic block copolymer instead of or in addition to one or more hydrophobic copolymers. Illustratively, the hydrophilic block copolymer may include one or more hydrophilic blocks selected from the group consisting of polyethylene glycol (PEG) and cellulosic polymers. Additionally, or alternatively, the block copolymer may include one or more hydrophobic blocks selected from the group consisting of polydimethylsiloxane (PDMS) polytetrafluoroethylene, polyethylene-co-tetrafluoroethylene, polyolefin, polyester, polycarbonate, bio stable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, poly(propylene oxide) and copolymers and blends thereof. In one example, the ion-selective membrane 2112 does not contain PVP, or other plasticizers.

In one example, the biocompatible polymer of the ion-selective membrane 1412 includes from about 0.1 wt. % silicone to about 80 wt. % silicone. In one example, the ion-selective membrane 1412, or other ion-selective membranes discussed herein, includes from about 5 wt. % silicone to about 25 wt. % silicone. In yet another example, the ion-selective membrane 1412, or other ion-selective membranes discussed herein, includes from about 35 wt. % silicone to about wt. % silicone. In yet another example, the ion-selective membrane 1412, or other ion-selective membranes discussed herein, includes from about 30 wt. % silicone to about 50 wt. % silicone.

In certain examples, the ISM 1412 or other ISMs discussed herein may include one or more block copolymers or segmented block copolymers. The segmented block copolymer may include hard segments and soft segments. In this example, the hard segments may include aromatic or aliphatic diisocyanates are used to prepare hard segments of segmented block copolymer. In one example, the aliphatic or aromatic diisocyanate used to provide hard segment of polymer includes one or more of norbornane diisocyanate (NBDI), isophorone diisocyanate (IPDI), tolylene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof. In one example, the hard segments may be from about 5 wt. % to about 90 wt. % of the segmented block copolymer of the ISM 1412. In another example, the hard segments may be from about 15 wt. % to about 75 wt. %. In yet another example, the hard segments may be from about 25 wt. % to about 55 wt. %. It will be appreciated that ion-selective membrane 1412 and first electrode 1311 may be prepared in any suitable manner. Illustratively, the polymer, ionophore 1315, and any additive may be dispersed in appropriate amounts in a suitable organic solvent (e.g., tetrahydrofuran, isopropyl alcohol, acetone, or methyl ethyl ketone). The mixture may be coated onto substrate 1301 (or onto a layer thereon) using any suitable technique, such as dipping and drying, spray-coating, inkjet printing, aerosol jet dispensing, slot-coating, electrodeposition, electrospraying, electrospinning, chemical vapor deposition, plasma polymerization, physical vapor deposition, spin-coating, or the like. The organic solvent may be removed so as to form a solid material corresponding to ion-selective membrane 1412 or first electrode 1311. Other layers in device 1300 or device 1400, such as electrodes, solid contact layers, and/or biological membranes, may be formed using techniques described elsewhere herein or otherwise known in the art.

Whereas first electrode 1311 includes a conductive polymer so as to be able to provide ionophore 1315 therein while retaining the electrical conductivity of an electrode, additional types of materials may be used in first electrode 1411 because an ionophore need not be provided therein. Nonlimiting example materials for use in first electrode 1411 of device 1400 are provided above with reference to second electrode 1317, e.g., a metal, a metal alloy, a transition metal oxide, a transparent conductive oxide, a carbon material, a doped semiconductor, a binary semiconductor, a ternary semiconductor, or a conductive polymer such as described above with reference to FIG. 13A.

In some examples, the ion-selective membrane is in direct contact with the first electrode. In other examples, such as illustrated in FIG. 14, sensor 1410 further may include a solid contact layer 1413 disposed between the first electrode 1411 and the ion-selective membrane 1412. Solid contact layer 1413 may perform the function of enhancing the reproducibility and stability of the EMF by converting the signal into a measurable electrical potential signal. Additionally, or alternatively, solid contact layer 1413 may inhibit transport of water from the biological fluid 10 to the first electrode 1411 and/or accumulation of water at the first electrode 1411. Solid contact layer 1413 may include any suitable material or combination of materials. Nonlimiting example materials for use in solid contact layer 1413 are provided above with reference to second electrode 1317, e.g., a metal, a carbon material, a doped semiconductor, or a conductive polymer such as described above with reference to FIG. 13A. Alternatively, solid contact layer 1413 may include a redox couple which has a well-controlled concentration ratio of oxidized/reduced species that may be used to stabilize the interfacial electrical potential. The redox couple may include metallic centers with different oxidation states. Illustratively, the metallic centers may be selected from the group consisting of Co(II) and Co(III); Ir(II) and Ir(III); and Os(II) and Os(III). In alternative examples, the solid contact layer 213 may include a mixed conductor, or mixed ion-electron conductor, such as strontium titanate (SrTiO₃), titanium dioxide (TiO₂), (La,Ba,Sr)(Mn,Fe,Co)O_{3-d},La₂CuO_{4+d}, cerium(IV) oxide (CeO₂), lithium iron phosphate (LiFePO₄), and LiMnPO₄.

It will further be appreciated that sensor 1410 may have any suitable configuration. In the nonlimiting example illustrated in FIG. 14, substrate 1301 may be planar or substantially planar.

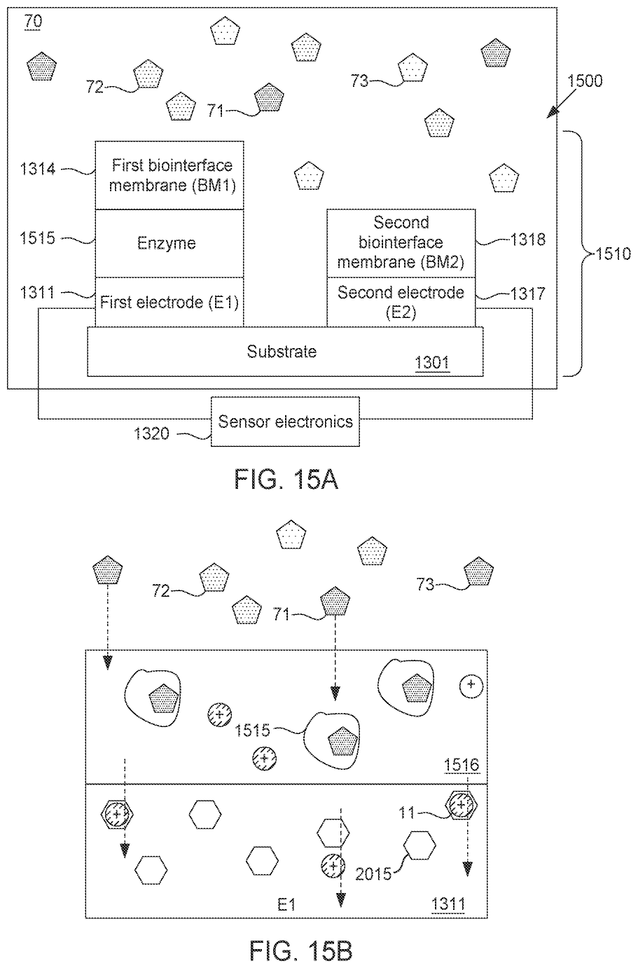

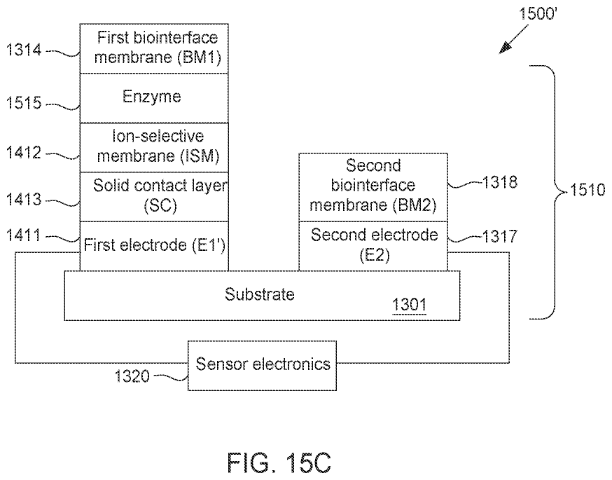

In the nonlimiting example illustrated in FIG. 15A, the ionophore may be located within first electrode (E1) 1311 disposed on the substrate and may be configured similarly as described with reference to FIG. 13A. Alternatively, in the nonlimiting example illustrated in FIG. the ionophore may be located within ion-selective membrane 1412 which may be configured in a manner such as described with reference to FIG. 14, and the first electrode 1411 may be configured in a manner such as described with reference to FIG. 14. First electrode 1311 or 1411 may be referred to as a working electrode (WE), while second electrode 1317 may be referred to as a reference electrode (RE).

The sensor electronics 1320 may be configured to generate a signal corresponding to an electromotive force (EMF). In some examples, the EMF is at least partially based on a potential difference that is generated between the first electrode and the second electrode responsive to the ionophore transporting the target ion to the first electrode. The sensor electronics 1320 may be configured to use the signal to generate an output corresponding to a measurement of the concentration of the target ion in the biological fluid, and/or may be configured to transmit the signal to an external device configured to use the signal to generate an output corresponding to a measurement of the concentration of the target ion in the biological fluid. Optionally, in some examples, the EMF is at least partially based on a potential difference that is generated between the first electrode and the second electrode responsive to biological fluid 10 conducting the electrophysiological signal to first electrode 111, and sensor electronics 1320 may be configured to use the signal to generate an output corresponding to a measurement of the electrophysiological signal.

In a manner such as illustrated in FIG. 15A, biological fluid 10 may include a plurality of analytes 71, 72, and 73. Device 1500 may be configured to measure the concentration of analyte 71, and accordingly such analyte may be referred to as a “target” analyte. As illustrated in FIG. 15B, enzyme 1515 may be located within enzyme layer 1516, and may selectively act upon target analyte 71 from biological fluid 10 or from biointerface membrane 1314 (if provided, e.g., as illustrated in FIG. 15A and configured similarly as described with reference to FIGS. 13A and 14). The action of enzyme 1515 upon the target analyte 71 generates the target ion 11. Ionophore 1315 within first electrode 1311 or within ion-selective membrane 1412 may selectively transport, or selectively bind, target ions 11 from enzyme 1515 to and within first electrode 1311 or first electrode 1411.

It will be appreciated that target analyte 71 may be any suitable analyte, enzyme 1515 may be any suitable enzyme that generates a suitable ion responsive to action upon that analyte, and ionophore 1315 may be any suitable ionophore that selectively transports and/or binds that ion generated by enzyme 1515 so as to generate an EMF based upon which the concentration of analyte 71 may be determined (whether using sensor electronics 1320 or an external device to which the sensor electronics 1320 transmits the electrophysiological signal and/or signal corresponding to ion concentration). Nonlimiting examples of analytes, enzymes, and ionophores that may be used together are listed below in Table 1.

TABLE 1

| Analyte | Enzyme | Ion generated | Ionophore |
|---|---|---|---|
| Urea | Urease | Ammonium | Nonactin |
| Glucose | Glucose oxidase | H+ (via | Tridodecylamine, 4- |
| | | peroxide) | Nonadecylpyridine, N,N- |
| | | | Dioctadecylmethylamine, |
| | | | Octadecyl isonicotinate, |
| | | | Calix[4]-aza-crown |
| Creatinine | Creatinine | Ammonium | Nonactin |
| | deaminase | | |
| Lactate | Lactate oxidase | H+ (via | Tridodecylamine, 4- |
| | | peroxide) | Nonadecylpyridine, N,N- |
| | | | Dioctadecylmethylamine, |
| | | | Octadecyl isonicotinate, |
| | | | Calix[4]-aza-crown |
| Cholesterol | Cholesterol oxidase | H+ (via | Tridodecylamine, 4- |
| | | peroxide) | Nonadecylpyridine, N,N- |
| | | | Dioctadecylmethylamine, |
| | | | isonicotinate, |
| | | | Octadecyl isonicotinate, |
| | | | Calix[4]-aza-crown |
| Glutamate | Glutamate oxidase/ | Ammonium | Nonactin |
| | Glutamate | | |
| | dehydrogenase | | |
| Galactose | Galactose/oxidase | H+ (via | Tridodecylamine, 4- |
| | | peroxide) | Nonadecylpyridine, N,N- |
| | | | Dioctadecylmethylamine, |
| | | | Octadecyl isonicotinate, |
| | | | Calix[4]-aza-crown |

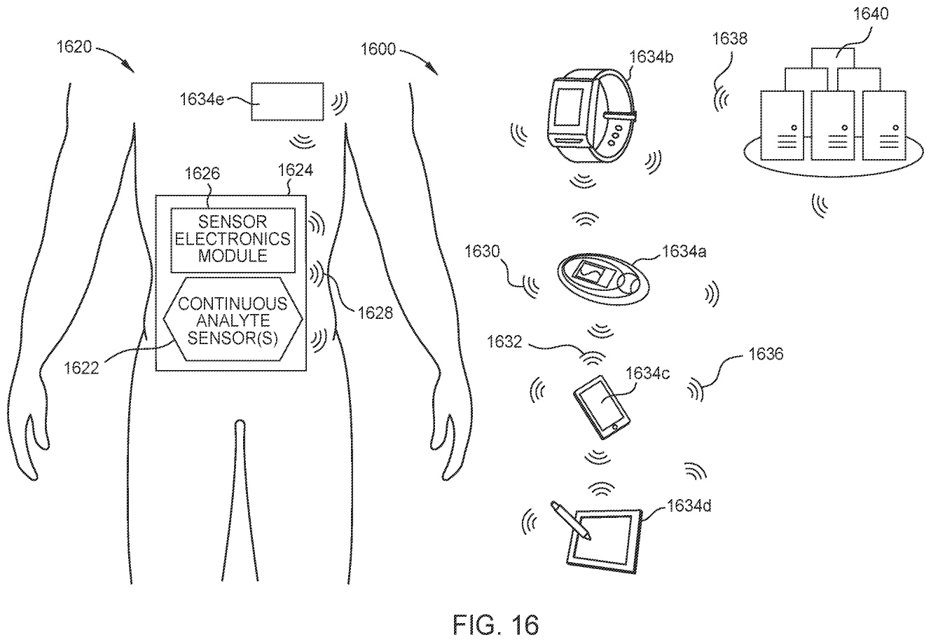

FIG. 16 is a diagram depicting an example continuous analyte monitoring system 1600 configured to measure one or more target ions and/or other analytes as discussed herein. The monitoring system 1600 includes an analyte sensor system 1624 operatively connected to a host 1620 and a plurality of display devices 1634 a-e according to certain aspects of the present disclosure. It should be noted that the display device 1634e alternatively or in addition to being a display device, may be a medicament delivery device that can act cooperatively with the analyte sensor system 1624 to deliver medicaments to host 1620. The analyte sensor system 1624 may include a sensor electronics module 1626 and a continuous analyte sensor 1622 associated with the sensor electronics module 1626. The sensor electronics module 1626 may be in direct wireless communication with one or more of the plurality of the display devices 1634a-e via wireless communications signals.

As will be discussed in greater detail below, display devices 1634a-e may also communicate amongst each other and/or through each other to analyte sensor system 1624. For ease of reference, wireless communications signals from analyte sensor system 1624 to display devices 1634a-e can be referred to as “uplink” signals 1628. Wireless communications signals from, e.g., display devices 1634a-e to analyte sensor system 1624 can be referred to as “downlink” signals 1630. Wireless communication signals between two or more of display devices 1634a-e may be referred to as “crosslink” signals 1632. Additionally, wireless communication signals can include data transmitted by one or more of display devices 1634a-d via “long-range” uplink signals 1636 (e.g., cellular signals) to one or more remote servers 1640 or network entities, such as cloud-based servers or databases, and receive long-range downlink signals 1638 transmitted by remote servers 1640.

The sensor electronics module 1626 includes sensor electronics that are configured to process sensor information and generate transformed sensor information. In certain embodiments, the sensor electronics module 1626 includes electronic circuitry associated with measuring and processing data from continuous analyte sensor 1622, including prospective algorithms associated with processing and calibration of the continuous analyte sensor data. The sensor electronics module 1626 can be integral with (non-releasably attached to) or releasably attachable to the continuous analyte sensor 1622 achieving a physical connection therebetween. The sensor electronics module 1626 may include hardware, firmware, and/or software that enables analyte level measurement. For example, the sensor electronics module 1626 can include a potentiostat, a power source for providing power to continuous analyte sensor 1622, other components useful for signal processing and data storage, and a telemetry module for transmitting data from itself to one or more display devices 1634a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 1634a-e are configured for displaying, alarming, and/or basing medicament delivery on the sensor information that has been transmitted by the sensor electronics module 1626 (e.g., in a customized data package that is transmitted to one or more of display devices 1634a-e based on their respective preferences). Each of the display devices 1634a-e can include a display such as a touchscreen display for displaying sensor information to a user (most often host 1620 or a caretaker/medical professional) and/or receiving inputs from the user. In some embodiments, the display devices 1634a-e may include other types of user interfaces such as a voice user interface instead of or in addition to a touchscreen display for communicating sensor information to the user of the display device 1634a-e and/or receiving user inputs. In some embodiments, one, some or all of the display devices 1634a-e are configured to display or otherwise communicate the sensor information as it is communicated from the sensor electronics module 1626 (e.g., in a data package that is transmitted to respective display devices 1634a-e), without any additional prospective processing required for calibration and real-time display of the sensor information.

In the embodiment of FIG. 16, one of the plurality of display devices 1634a-e may be a custom display device 1634a specially designed for displaying certain types of displayable sensor information associated with analyte values received from the sensor electronics module 1626 (e.g., a numerical value and an arrow, in some embodiments). In some embodiments, one of the plurality of display devices 1634a-e may be a handheld device 1634c, such as a mobile phone based on the Android, iOS operating system or other operating system, a palm-top computer and the like, where handheld device 1634c may have a relatively larger display and be configured to display a graphical representation of the continuous sensor data (e.g., including current and historic data). Other display devices can include other hand-held devices, such as a tablet 1634d, a smart watch 1634b, a medicament delivery device 1634e, a blood glucose meter, and/or a desktop or laptop computer.

As discussed above, because the different display devices 1634a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the embodiment of FIG. 13A, one or more of display devices 1634a-e can be in direct or indirect wireless communication with the sensor electronics module 1626 to enable a plurality of different types and/or levels of display and/or functionality associated with the sensor information, which is described in more detail elsewhere herein.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, acc, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
a substrate,
a working electrode disposed on the substrate,
a reference electrode disposed on the substrate, wherein the analyte measurements generated by the continuous analyte sensor correspond to an electromotive force at least in part based on a potential difference generated between the working electrode and the reference electrode.

3. The monitoring system of claim 1, wherein:
the continuous analyte sensor comprises a continuous glucose sensor, and
the analyte measurements include glucose measurements.

4. The monitoring system of claim 3, further comprising:
a memory comprising executable instructions; and
one or more processors in data communication with the memory and configured to execute the executable instructions to:
receive glucose data associated with the glucose measurements from the sensor electronics module;
process the glucose data to determine at least one glucose clearance rate for the patient based on the glucose data;
determine a likelihood of at least one atypical glucose trend associated with the at least one glucose clearance rate; and
generate decision support output based on the determined likelihood.

5. The monitoring system of claim 4, further comprising:
one or more non-analyte sensors, wherein the processor is further configured to:
receive non-analyte sensor data generated for the patient using one or more non-analyte sensors, wherein the determined likelihood of at least one atypical glucose trend is further based on the non-analyte sensor data.

6. The monitoring system of claim 5, wherein the one or more non-analyte sensors comprise at least one of an insulin pump, a haptic sensor, an ECG sensor, a heart rate monitor, a blood pressure sensor, a respiratory sensor, a peritoneal dialysis machine, or a hemodialysis machine.

7. The monitoring system of claim 4, wherein the likelihood of the at least one atypical glucose trend is indicative of a risk of hyperglycemia or hypoglycemia.

8. The monitoring system claim 4, wherein the decision support output comprises at least one of:
an alert of an adverse glycemic event;
a recommendation for treatment; and
a recommendation for prevention of the at least one atypical glucose trend.

9. The monitoring system of claim 3, further comprising:
a memory comprising executable instructions;
one or more processors in data communication with the memory and configured to execute the executable instructions to:
receive glucose data associated with the glucose measurements from the sensor electronics module;
process the glucose data to determine at least one glucose metric for the patient based on the glucose data; and
generate a diabetes disease prediction based on the at least one glucose metric.

10. The monitoring system of claim 9, wherein the processor is further configured to generate one or more recommendations for treatment based, at least in part, on the diabetes disease prediction.

11. The monitoring system of claim 9, further comprising:
one or more non-analyte sensors, wherein the processor is further configured to:
receive non-analyte sensor data generated for the patient using one or more non-analyte sensors, wherein the diabetes disease prediction is further generated based on the non-analyte sensor data.

12. The monitoring system of claim 11, wherein the one or more non-analyte sensors comprise at least one of an insulin pump, a haptic sensor, an ECG sensor, a heart rate monitor, a blood pressure sensor, a respiratory sensor, a peritoneal dialysis machine, or a hemodialysis machine.

13. The monitoring system of claim 9, wherein the diabetes disease prediction is indicative of a risk of developing diabetes or a current diabetes diagnosis of the patient.

14. The monitoring system of claim 9, wherein the diabetes disease prediction is generated using a model trained based on population data including records of historical patients with varying stages of diabetes.

15. The monitoring system of claim 10, wherein the recommendations for treatment include at least one of: a lifestyle recommendation, a medication recommendation, or a medical intervention recommendation.
